(19) United States
(12) Patent Application Publication
(10) Pub. No.: US 2014/0038989 A1
(43) Pub. Date: Feb. 6, 2014

(54) TETRAHYDROPYRAZOLO [1,5-A] PYRIMIDINE AS ANTI-TUBERCULOSIS COMPOUNDS

(71) Applicants: Emilio Alvarez-Ruiz, Tres cantos (ES); Luis Ballell-Pages, Tres cantos (ES); Julia Castro Pichel, Tres cantos (ES); Lourdes Encinas, Tres cantos (ES); Jorge Esquivias, Tres cantos (ES); Franscisco Gamo-Benito, Tres cantos (ES); Maria Cruz Garcia-Palancar, Tres cantos (ES); Modesto Jesus Remuinan-Blanco, Tres cantos (ES)

(72) Inventors: Emilio Alvarez-Ruiz, Tres cantos (ES); Luis Ballell-Pages, Tres cantos (ES); Julia Castro Pichel, Tres cantos (ES); Lourdes Encinas, Tres cantos (ES); Jorge Esquivias, Tres cantos (ES); Franscisco Gamo-Benito, Tres cantos (ES); Maria Cruz Garcia-Palancar, Tres cantos (ES); Modesto Jesus Remuinan-Blanco, Tres cantos (ES)

(73) Assignee: Glaxo Group Limited, Brentford Middlesex (GB)

(21) Appl. No.: 14/112,564

(22) PCT Filed: Apr. 20, 2012
(86) PCT No.: PCT/EP2012/057302

§ 371 Date: Oct. 18, 2013

(30) Foreign Application Priority Data
Apr. 20, 2011 (EP) 11382119.3
Mar. 2, 2012 (EP) 12382081.3

(51) Int. Cl. C07D 487/04; A61K 45/06; A61K 31/519

(52) U.S. Cl. CPC C07D 487/04; A61K 31/519; A61K 45/06
USPC 514/259.3; 544/281

(57) ABSTRACT

A compound of Formula (I) or a pharmaceutically acceptable salt thereof:

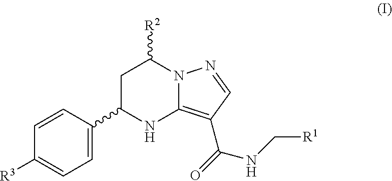

wherein:
R¹ represents a group selected from:
i) phenyl optionally substituted with one or two substituents independently selected from Me, OMe, CF₃, F, Cl and NMe₂;
furanyl, thiophenyl, pyrrolyl, pyridyl, cyclohexyl or naphthyl, each of which is optionally substituted with one or two substituents independently selected from Me, OMe, CF₃, F, Cl and NMe₂; and
iii) benzo[1,3]dioxo5-yl or 2,3-dihydrobenzo[1,4]dioxin-6-yl;
   R² represents CF₃, C₁₋₄alkyl, or CHF₂;
   when R¹ represents optionally substituted furanyl, thiophenyl, pyrrolyl, pyridyl or naphthyl, R³ represents Et;
   when R¹ represents optionally substituted cyclohexyl, R³ represents Et or Me;
   otherwise R³ represents Et, Me, Br or OMe,
   compositions containing them, their use in therapy, for example in the treatment of tuberculosis, and methods for the preparation of such compounds, are provided, together with certain novel compounds.

### FIELD OF THE INVENTION

This invention relates to compounds, compositions containing them, their use in therapy, for example in the treatment of tuberculosis, and methods for the preparation of such compounds.

### BACKGROUND OF THE INVENTION

Synthetic drugs for treating tuberculosis (TB) have been available for over half a century, but incidences of the disease continue to rise world-wide. In 2004, it is estimated that 24,500 people developed active disease and close to 5,500 died each day from TB (World Health Organization, Global Tuberculosis Control: Surveillance, Planning, Financing. WHO Report 2006, Geneva, Switzerland, ISBN 92-4 156314-1). The threat this disease represents is still a painful reality for the ten million people now infected, and for the two million that die each year (WHO Report 2007: Global Tuberculosis Control Report). Co-infection with HIV is driving the increase in incidence (Williams, B. G.; Dye, C. Science, 2003, 301, 1535) and the cause of death in 31% of AIDS patients in Africa can be attributed to TB (Corbett, E. L.; Watt, C. J.; Catherine, J.; Walker, N.; Maher D.; Williams, B. G.; Raviglione, M. C.; Dye, C. Arch. Intl. Med., 2003, 163, 1009, Septkowitz, A.; Raffalli, J.; Riley, T.; Kiehn, T. E.; Armstrong, D. Clin. Microbiol. Rev. 1995, 8, 180). When coupled with the emergence of multi-drug resistant strains of Mycobacterium tuberculosis, the scale of the problem is amplified. It is now more than a decade since the WHO declared TB “a global health emergency” (World Health Organization, Global Tuberculosis Control: Surveillance, Planning, Financing. WHO Report 2006, Geneva, Switzerland, ISBN 92-4 156314-1).

The limitations of tuberculosis therapy and prevention are well-known. The current available vaccine, BCG was introduced in 1921 and fails to protect most people past childhood. Patients who do become infected with active disease currently endure combination therapy with isoniazid (INH), rifampin, pyrazinamide and ethambutol for two months and then continue taking isoniazid and rifampin for a further four months; furthermore, daily dosing is required. This first-line drug treatment is effective in active, drug-susceptible TB as long as patients complete the course. However, there is a poor patient compliance due to many factors such as the cost of drugs, side effects, long time necessary for full treatment and the number of drug doses required (Current Medicinal Chemistry, 2008, 15, 1956-1967). In addition, poor patient compliance drives the emergence and spread of Multi-Drug Resistant (MDR) TB strains, which are challenging to treat. Multi-drug resistant TB strains are resistant to at least the two main first-line TB drugs—isoniazid and rifampicin; and Extremely Drug Resistant (XDR) TB, strains are also resistant to three or more of the six classes of second-line drugs. In some Eastern Europe Central Asia Countries (EECAC) such as Azerbaijan, MDR/XDR strains can account for up to 22% of infections, with mortality rates for XDR reaching up to 100% of those infected (Eur. Respir. J., 33, 2009, 871). A recently-published detailed review discusses many aspects of TB such as pathogenesis, epidemiology, drug discovery and vaccine development to date (Nature Medicine, Vol 13(3), pages 263-312).

Shorter courses of more active agents which can be taken less frequently and which present a high barrier to the emergence of resistance, i.e. agents which are effective against multi-drug resistant strains of TB, are urgently required. An additional problem is the drug-drug interaction of current TB drugs with other disease treatments, like HIV or diabetes. There is therefore an urgent need to develop new chemical entities to treat TB,

focused particularly on: a) shortening current therapy of six months; b) activity against MDR-XDR strains; c) possible co-administration with other medications, for example by targeting novel biochemical pathways. Recent synthetic leads are reviewed in: Ballell, L.; Field, R. A.; Duncan, K.; Young, R. J. Antimicrob. Agents Chemother. 2005, 49, 2153. Results from the screening of a compound library against Mycobacterium tuberculosis strain H37Rv, a have been reported (Tuberculosis, 2009, 89, 354-363).

### DETAILED DESCRIPTION OF THE INVENTION

The present invention provides a compound of Formula (I) or a pharmaceutically acceptable salt thereof:

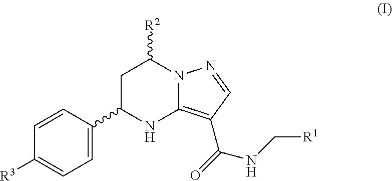

### Wherein:

R¹ represents a group selected from:
i) phenyl optionally substituted with one or two substituents independently selected from Me, OMe, CF₃, F, Cl and NMe₂;
ii) furanyl, thiophenyl, pyrrolyl, pyridyl, cyclohexyl or naphthyl, each of which is optionally substituted with one or two substituents independently selected from Me, OMe, CF₃, F, Cl and NMe₂;
and

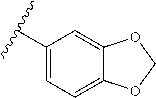 [SMILES: *c1ccc2c(c1)OCO2]

iii) benzo[1,3]dioxo5-yl:
   or 2,3-dihydrobenzo[1,4]dioxin-6-yl:

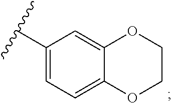 [SMILES: *c1ccc2c(c1)OCCO2]

R² represents CF₃, C₁₋₄alkyl, or CHF₂; and
When R¹ represents optionally substituted furanyl, thiophenyl, pyrrolyl, pyridyl or naphthyl, R³ represents Et;
When R¹ represents optionally substituted cyclohexyl, R³ represents Et or Me; Otherwise R³ represents Et, Me, Br or OMe;
for use in the treatment of tuberculosis.

In one aspect of the invention there is provided a compound of Formula (IA) or a pharmaceutically acceptable salt thereof:

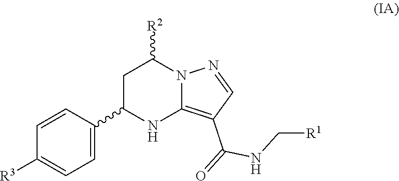

### Wherein:

R¹ represents a group selected from:
i) phenyl optionally substituted with one or two substituents independently selected from Me, OMe, CF₃, F and NMe₂, or phenyl substituted with either a) one Cl substituent at the 4-position, or b) two Cl substituents at the 3- and 4-positions;
ii) furanyl, thiophenyl, pyrrolyl, pyridyl or naphthyl, each of which is optionally substituted with one or two substituents independently selected from Me, OMe, CF₃, F, Cl and NMe₂; or cyclohexyl substituted with one or two substituents independently selected from Me, OMe, CF₃, F, Cl and NMe₂;
and

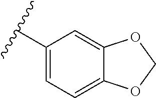 [SMILES: *c1ccc2c(c1)OCO2]

iii) benzo[1,3]dioxo5-yl:
   or 2,3-dihydrobenzo[1,4]dioxin-6-yl:

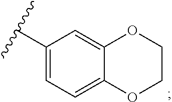 [SMILES: *c1ccc2c(c1)OCCO2]

R² represents CF₃, C₁₋₄alkyl, or CHF₂; and
When R¹ represents optionally substituted furanyl, thiophenyl, pyrrolyl, pyridyl or naphthyl,
R³ represents Et;
When R¹ represents substituted cyclohexyl, R³ represents Et or Me;
Otherwise R³ represents Et, Me, Br or OMe;
for use in therapy.

In one aspect of the invention there is provided a compound of Formula (IB) or a pharmaceutically acceptable salt thereof:

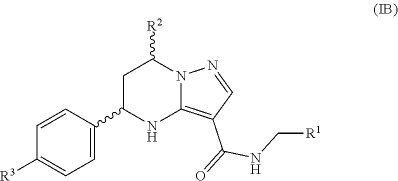

### Wherein:

R¹ represents a group selected from:
i) phenyl optionally substituted with one or two substituents independently selected from Me, CF₃, F and NMe₂, or phenyl substituted with either a) one Cl substituent at the 4-position, or b) two Cl substituents at the 3- and 4-positions; ii) furanyl, pyrrolyl, pyridyl or naphthyl, each of which is optionally substituted with one or two substituents independently selected from Me, OMe, CF₃, F, Cl and NMe₂; or cyclohexyl or thiophenyl, each of which is substituted with one or two substituents independently selected from Me, OMe, CF₃, F, Cl and NMe₂;
and

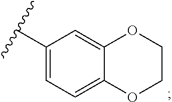 [SMILES: *c1ccc2c(c1)OCCO2]

iii) 2,3-dihydrobenzo[1,4]dioxin-6-yl:
   R² represents CF₃, C₁₋₄alkyl, or CHF₂; and
   When R¹ represents optionally substituted furanyl, pyrrolyl, pyridyl or naphthyl, or substituted thiophenyl or when R² represents CHF₂, R³ represents Et;
   When R¹ represents substituted cyclohexyl, R³ represents Et or Me;
   Otherwise R³ represents Et, Me or Br.

The invention further provides an enantiomeric compound of formula (IC) or a pharmaceutically acceptable salt thereof:

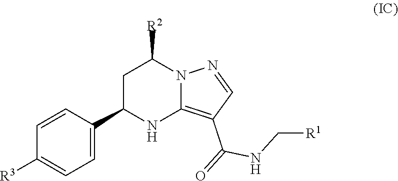

### Wherein

R¹ represents a group selected from:
(i) phenyl optionally substituted with one or two substituents independently selected from Me, OMe, CF₃, F, Cl and NMe₂;
(ii) furanyl, thiophenyl, pyrrolyl, pyridyl, cyclohexyl or naphthyl, each of which is optionally substituted with one or two substituents independently selected from Me, OMe, CF₃, F, Cl and NMe₂;
and
(iii) benzo[1,3]dioxo5-yl:

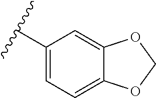 [SMILES: *c1ccc2c(c1)OCO2]

or 2,3-dihydrobenzo[1,4]dioxin-6-yl:

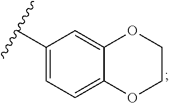 [SMILES: *c1ccc2c(c1)OCCO2]

R² represents CF₃, C₁₋₄alkyl, or CHF₂; and
R³ represents Et, Me, Br or OMe.

In one embodiment R¹ is selected from phenyl substituted with one or two substituents independently selected from Me, OMe, and F; pyridyl substituted with one or two substituents independently selected from Me, and F; furanyl substituted with methyl; and benzo[1,3]dioxo5-yl:

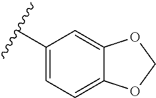 [SMILES: *c1ccc2c(c1)OCO2]

In one embodiment, when R¹ represents optionally substituted furanyl, thiophenyl, pyrrolyl, pyridyl or naphthyl, R³ represents Et;

When R¹ represents optionally substituted cyclohexyl, R³ represents Et or Me;

Otherwise R³ represents Et, Me, Br or OMe;

In one embodiment R¹ is phenyl substituted with one or two substituents independently selected from Me, OMe, and F.

In one embodiment R¹ is pyridyl substituted with one or two substituents independently selected from Me, and F.

In one embodiment R¹ is furanyl substituted with methyl.

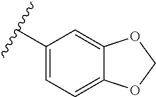 [SMILES: *c1ccc2c(c1)OCO2]

In one embodiment R¹ is benzo[1,3]dioxo5-yl:

In one embodiment R² is CF₃, or In one embodiment R² is CF₃

In one embodiment R² is CH₃

In one embodiment R³ is Et.

References herein to a compound of Formula (I) will be understood to include a compound of Formula (IA) or (IB) or (IC).

In one aspect of the invention there is provided a compound of Formula (I) as defined hereinabove for use in the treatment of tuberculosis.

In one aspect of the invention there is provided a compound of Formula (IA) as defined hereinabove for use in therapy.

In one aspect of the invention there is provided a compound of Formula (IB) as defined hereinabove.

In one aspect of the invention there is provided a compound of Formula (IC) as defined above.

The invention further provides a pharmaceutical composition comprising a compound of Formula (I), or a pharmaceutically acceptable salt thereof, and one or more pharmaceutically acceptable carriers, excipients or diluents.

The invention also provides a method of treatment of tuberculosis in mammals, particularly in man, which method comprises the administration to a mammal in need of such treatment an effective amount of a compound of Formula (I), or a pharmaceutically acceptable salt thereof.

The invention further provides a compound of Formula (I), or a pharmaceutically acceptable salt thereof, for use in therapy.

The invention yet further provides a compound of Formula (I), or a pharmaceutically acceptable salt thereof, for use in the treatment of tuberculosis in mammals, particularly in man.

The invention still further provides the use of a compound of Formula (I), or a pharmaceutically acceptable salt thereof, in the manufacture of a medicament for use in the treatment of tuberculosis in mammals, particularly in man.

The invention also provides a pharmaceutical composition comprising a compound of Formula (I), or a pharmaceutically acceptable salt thereof, and one or more pharmaceutically acceptable carriers, excipients or diluents, for use in the treatment of tuberculosis in mammals, particularly in man.

It will be appreciated that in the aforementioned aspects of the invention a compound of Formula (I) (IA) (IB) or (IC) may be in the form of a pharmaceutically acceptable salt or as a free base. In one embodiment of said aspects the compound of a compound of Formula (I) (IA) (IB) or (IC) is in the form of the free base.

In one aspect the invention provides a process for the preparation of a compound of Formula (I) comprising the step of reacting a compound of Formula (II), wherein R² and R³ are as described herein for Formula (I), with an amine R¹CH₂NH₂, wherein R¹ is as described herein for Formula (I), or with a salt of such an amine (e.g. the hydrochloride salt). In another aspect, the compound of Formula (II) is reacted with an amine R¹CH₂NH₂, or with a salt of such an amine, under suitable conditions for making an amide bond. In a further aspect, the compound of Formula (II) is reacted with an amine R¹CH₂NH₂, or with a salt of such an amine, in the presence of a base (e.g. N,N-diisopropylethylamine or triethylamine) and a coupling reagent (e.g. HATU, HOBt or EDC). In a yet further aspect, the compound of Formula (II) is reacted with an amine R¹CH₂NH₂, or with a salt of such an amine, in the presence of a base (e.g. N,N-diisopropylethylamine or triethylamine) and a coupling reagent (e.g. HATU, HOBt or EDC) and a suitable solvent (e.g. DMF or DCM) at elevated temperature (e.g. 60° C.).

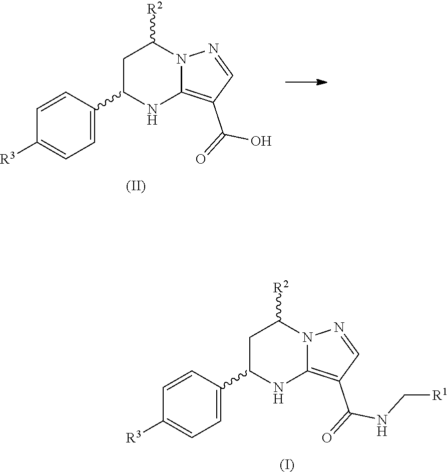

In one aspect of the invention, the relative stereochemistry of the compound of Formula (I), or a pharmaceutically acceptable salt thereof, is cis, as shown in Formula (I*):

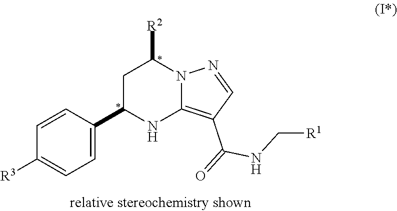

It will be appreciated by the skilled artisan that the compound of Formula (I) possesses two stereogenic centres (labelled * in Formula (I*) above). The compound of Formula (I) may be in the form of a mixture of isomers, for example a racemic mixture of enantiomers, or in the form of a single isomer, for example, an enantiomer in at least 95% enantiomeric excess (e.e.). In one embodiment, the invention provides a single enantiomer of a compound of Formula (I) or a pharmaceutically acceptable salt thereof. In a further embodiment, the invention provides a compound of Formula (I) or a pharmaceutically acceptable salt thereof having the relative stereochemistry shown in Formula (I*).

In one aspect of the invention, R¹ represents phenyl optionally substituted with one or two substituents independently selected from Me, OMe, CF₃, F, Cl and NMe₂. In another aspect, R¹ represents phenyl optionally substituted with either a) one substituent at either the 3- or the 4-position, or b) two substituents at the 3- and 4-positions, the optional substituents being independently selected from Me, OMe, CF₃, F, Cl and NMe₂. In another aspect, R¹ represents phenyl optionally substituted with one or two substituents independently selected from Me, OMe, CF₃, F and NMe₂, or phenyl substituted with either a) one Cl substituent at the 4-position, or b) two Cl substituents at the 3- and 4-positions. In a further aspect, R¹ represents phenyl optionally substituted with one or two substituents independently selected from Me, CF₃, F, Cl and NMe₂. In a further aspect, R¹ represents phenyl optionally substituted with one or two substituents independently selected from Me, OMe, CF₃, F, and Cl.

In one aspect of the invention, R¹ represents furanyl, thiophenyl, pyrrolyl, pyridyl, cyclohexyl or naphthyl, each of which is optionally substituted with one or two substituents independently selected from Me, OMe, CF₃, F, Cl and NMe₂. In another aspect of the invention, R¹ represents furanyl, thiophenyl, pyrrolyl, pyridyl, cyclohexyl or naphthyl, each of which is optionally substituted with Me. In another aspect of the invention, R¹ represents furanyl, thiophenyl, pyrrolyl, pyridyl, cyclohexyl or naphthyl, each of which is unsubstituted. In a further aspect, R¹ represents furanyl, thiophenyl, pyrrolyl, pyridyl or naphthyl, each of which is optionally substituted with one or two substituents independently selected from Me, OMe, CF₃, F, Cl and NMe₂; or cyclohexyl substituted with one or two substituents independently selected from Me, OMe, CF₃, F, Cl and NMe₂. In a yet further aspect, R¹ represents furanyl, pyrrolyl, pyridyl or naphthyl, each of which is optionally substituted with one or two substituents independently selected from Me, OMe, CF₃, F, Cl and NMe₂; or cyclohexyl or thiophenyl, each of which is substituted with one or two substituents independently selected from Me, OMe, CF₃, F, Cl and NMe₂. In a further aspect, R¹ represents furanyl, thiophenyl, or pyridyl, each of which is optionally substituted with one or two substituents independently selected from Me, OMe, CF₃, F, and Cl.

In another aspect of the invention, R¹ represents benzo[1,3]dioxo5-yl or 2,3-dihydrobenzo[1,4]dioxin-6-yl. In a further aspect, R¹ represents 2,3-dihydrobenzo[1,4]dioxin-6-yl. In a further aspect, R¹ represents benzo[1,3]dioxo5-yl.

In one aspect of the invention, R² represents CF₃, t-Bu, i-Pr, Me or CHF₂. In one aspect of the invention, R² represents CF₃, t-Bu, Me or CHF₂. In another aspect, R² represents CF₃.

In one aspect of the invention, when R¹ represents optionally substituted phenyl or benzo[1,3]dioxo5-yl or 2,3-dihydrobenzo[1,4]dioxin-6-yl, R³ represents Et, Me, Br or OMe.

In another aspect, when R¹ represents optionally substituted phenyl or benzo[1,3]dioxo5-yl or 2,3-dihydrobenzo[1,4]dioxin-6-yl, R³ represents Et, Me or Br. In a further aspect, when R¹ represents optionally substituted phenyl or benzo[1,3]dioxo5-yl or 2,3-dihydrobenzo[1,4]dioxin-6-yl, R³ represents Et or Me. In another aspect, when R¹ represents optionally substituted phenyl or benzo[1,3]dioxo5-yl or 2,3-dihydrobenzo[1,4]dioxin-6-yl, R³ represents Et. In another aspect, when R¹ represents optionally substituted phenyl or 2,3-dihydrobenzo[1,4]dioxin-6-yl, R³ represents ethyl. In another aspect, R³ represents Et.

In one aspect of the invention, R¹ represents optionally substituted furanyl, thiophenyl, pyrrolyl, pyridyl or naphthyl, and R³ represents Et. In another aspect of the invention, R¹ represents optionally substituted furanyl, thiophenyl, or pyridyl, and R³ represents Et.

In one aspect of the invention, R¹ represents optionally substituted cyclohexyl, and R³ represents Et or Me. In another aspect of the invention, R¹ represents optionally substituted cyclohexyl, and R³ represents Et.

In one aspect of the invention, when R² represents CHF₂, R³ represents Et.

In one aspect, compounds which are useful in the present invention include those mentioned in the Examples and their pharmaceutically acceptable salts.

In another aspect, compounds which are useful in the present invention include the title compound of each of examples 1-41 and 58-106 herein.

In one aspect, the invention provides a compound of Formula (I) which is selected from:
Cis-5-(4-ethylphenyl)-N-(4-methylbenzyl)-7-(trifluoromethyl)-4,5,6,7-tetrahydropyrazolo[1,5-a]pyrimidine-3-carboxamide;
Cis-5-(4-ethylphenyl)-N-(3-methylbenzyl)-7-(trifluoromethyl)-4,5,6,7-tetrahydropyrazolo[1,5-a]pyrimidine-3-carboxamide;
Cis-5-(4-ethylphenyl)-N-((5-methylfuran-2-yl)methyl)-7-(trifluoromethyl)-4,5,6,7-tetrahydropyrazolo[1,5-a]pyrimidine-3-carboxamide;
Cis-5-(4-ethylphenyl)-7-(trifluoromethyl)-N-(4-(trifluoromethyl)benzyl)-4,5,6,7-tetrahydropyrazolo[1,5-a]pyrimidine-3-carboxamide;
Cis-5-(4-ethylphenyl)-N-(2-furanylmethyl)-7-(trifluoromethyl)-4,5,6,7-tetrahydropyrazolo[1,5-a]pyrimidine-3-carboxamide;
Cis-5-(4-ethylphenyl)-N-{[3-(methyloxy)phenyl]methyl}-7-(trifluoromethyl)-4,5,6,7-tetrahydropyrazolo[1,5-a]pyrimidine-3-carboxamide;
Cis-N-(3-chloro-4-methylbenzyl)-5-(4-ethylphenyl)-7-(trifluoromethyl)-4,5,6,7-tetrahydropyrazolo[1,5-a]pyrimidine-3-carboxamide;
Cis-5-(4-ethylphenyl)-N-[(1-methyl-1H-pyrrol-2-yl)methyl]-7-(trifluoromethyl)-4,5,6,7-tetrahydropyrazolo[1,5-a]pyrimidine-3-carboxamide;
Cis-N-(4-(dimethylamino)benzyl)-5-(4-ethylphenyl)-7-(trifluoromethyl)-4,5,6,7-tetrahydropyrazolo[1,5-a]pyrimidine-3-carboxamide;
Cis-5-(4-ethylphenyl)-N-(furan-3-ylmethyl)-7-(trifluoromethyl)-4,5,6,7-tetrahydropyrazolo[1,5-a]pyrimidine-3-carboxamide;
Cis-5-(4-ethylphenyl)-N-(1-naphthalenylmethyl)-7-(trifluoromethyl)-4,5,6,7-tetrahydropyrazolo[1,5-a]pyrimidine-3-carboxamide;
Cis-5-(4-ethylphenyl)-7-(trifluoromethyl)-N-{[3-(trifluoromethyl)phenyl]methyl}-4,5,6,7-tetrahydropyrazolo[1,5-a]pyrimidine-3-carboxamide;
Cis-N-benzyl-5-(4-ethylphenyl)-7-(trifluoromethyl)-4,5,6,7-tetrahydropyrazolo[1,5-a]pyrimidine-3-carboxamide;
Cis-N-[(3,4-difluorophenyl)methyl]-5-(4-ethylphenyl)-7-(trifluoromethyl)-4,5,6,7-tetrahydropyrazolo[1,5-a]pyrimidine-3-carboxamide;
Cis-N-[(3,5-difluorophenyl)methyl]-5-(4-ethylphenyl)-7-(trifluoromethyl)-4,5,6,7-tetrahydropyrazolo[1,5-a]pyrimidine-3-carboxamide;
Cis-N-(2,3-dihydro-1,4-benzodioxin-6-ylmethyl)-5-(4-ethylphenyl)-7-(trifluoromethyl)-4,5,6,7-tetrahydropyrazolo[1,5-a]pyrimidine-3-carboxamide;
Cis-5-(4-ethylphenyl)-N-((6-methylpyridin-3-yl)methyl)-7-(trifluoromethyl)-4,5,6,7-tetrahydropyrazolo[1,5-a]pyrimidine-3-carboxamide;
Cis-5-(4-ethylphenyl)-N-((5-methylpyridin-2-yl)methyl)-7-(trifluoromethyl)-4,5,6,7-tetrahydropyrazolo[1,5-a]pyrimidine-3-carboxamide;
Cis-5-(4-bromophenyl)-N-(4-methylbenzyl)-7-(trifluoromethyl)-4,5,6,7-tetrahydropyrazolo[1,5-a]pyrimidine-3-carboxamide;
Cis-N-(benzo[d][1,3]dioxol-5-ylmethyl)-5-(4-bromophenyl)-7-(trifluoromethyl)-4,5,6,7-tetrahydropyrazolo[1,5-a]pyrimidine-3-carboxamide;
Cis-5-(4-ethylphenyl)-7-methyl-N-{[4-(methyloxy)phenyl]methyl}-4,5,6,7-tetrahydropyrazolo[1,5-a]pyrimidine-3-carboxamide;
Cis-N-(3-methylbenzyl)-5-(p-tolyl)-7-(trifluoromethyl)-4,5,6,7-tetrahydropyrazolo[1,5-a]pyrimidine-3-carboxamide;
Cis-N-(4-methylbenzyl)-5-(p-tolyl)-7-(trifluoromethyl)-4,5,6,7-tetrahydropyrazolo[1,5-a]pyrimidine-3-carboxamide;
Cis-N-(cyclohexylmethyl)-5-(p-tolyl)-7-(trifluoromethyl)-4,5,6,7-tetrahydropyrazolo[1,5-a]pyrimidine-3-carboxamide;
Cis-N-(3-methoxybenzyl)-5-(p-tolyl)-7-(trifluoromethyl)-4,5,6,7-tetrahydropyrazolo[1,5-a]pyrimidine-3-carboxamide;
Cis-5-(p-tolyl)-7-(trifluoromethyl)-N-(4-(trifluoromethyl)benzyl)-4,5,6,7-tetrahydropyrazolo[1,5-a]pyrimidine-3-carboxamide;
Cis-N-(3-chloro-4-methylbenzyl)-5-(p-tolyl)-7-(trifluoromethyl)-4,5,6,7-tetrahydropyrazolo[1,5-a]pyrimidine-3-carboxamide;
Cis-N-(3,5-difluorobenzyl)-5-(p-tolyl)-7-(trifluoromethyl)-4,5,6,7-tetrahydropyrazolo[1,5-a]pyrimidine-3-carboxamide;
Cis-N-(3,4-difluorobenzyl)-5-(p-tolyl)-7-(trifluoromethyl)-4,5,6,7-tetrahydropyrazolo[1,5-a]pyrimidine-3-carboxamide;
Cis-7-(difluoromethyl)-5-(4-ethylphenyl)-N-(4-methoxybenzyl)-4,5,6,7-tetrahydropyrazolo[1,5-a]pyrimidine-3-carboxamide;
Cis-7-(difluoromethyl)-5-(4-ethylphenyl)-N-(4-methylbenzyl)-4,5,6,7-tetrahydropyrazolo[1,5-a]pyrimidine-3-carboxamide;
Cis-N-(benzo[d][1,3]dioxol-5-ylmethyl)-7-(difluoromethyl)-5-(4-ethylphenyl)-4,5,6,7-tetrahydropyrazolo[1,5-a]pyrimidine-3-carboxamide;
Cis-7-(tert-butyl)-5-(4-ethylphenyl)-N-(4-methoxybenzyl)-4,5,6,7-tetrahydropyrazolo[1,5-a]pyrimidine-3-carboxamide;
Cis-N-(benzo[d][1,3]dioxol-5-ylmethyl)-7-(tert-butyl)-5-(4-ethylphenyl)-4,5,6,7-tetrahydropyrazolo[1,5-a]pyrimidine-3-carboxamide;
Cis-5-(4-ethylphenyl)-N-(4-fluorobenzyl)-7-(trifluoromethyl)-4,5,6,7-tetrahydropyrazolo[1,5-a]pyrimidine-3-carboxamide;
Cis-N-(2,4-difluorobenzyl)-5-(4-ethylphenyl)-7-(trifluoromethyl)-4,5,6,7-tetrahydropyrazolo[1,5-a]pyrimidine-3-carboxamide;
Cis-5-(4-ethylphenyl)-N-(3-fluorobenzyl)-7-(trifluoromethyl)-4,5,6,7-tetrahydropyrazolo[1,5-a]pyrimidine-3-carboxamide;
Cis-5-(4-ethylphenyl)-N-((5-methylthiophen-2-yl)methyl)-7-(trifluoromethyl)-4,5,6,7-tetrahydropyrazolo[1,5-a]pyrimidine-3-carboxamide;
Cis-5-(4-ethylphenyl)-N-(2-fluorobenzyl)-7-(trifluoromethyl)-4,5,6,7-tetrahydropyrazolo[1,5-a]pyrimidine-3-carboxamide;
Cis-5-(4-ethylphenyl)-N-(4-fluoro-3-methylbenzyl)-7-(trifluoromethyl)-4,5,6,7-tetrahydropyrazolo[1,5-a]pyrimidine-3-carboxamide;
Cis-5-(4-ethylphenyl)-N-(3-fluoro-4-methylbenzyl)-7-(trifluoromethyl)-4,5,6,7-tetrahydropyrazolo[1,5-a]pyrimidine-3-carboxamide;
Cis-N-(3,4-dimethylbenzyl)-5-(4-ethylphenyl)-7-(trifluoromethyl)-4,5,6,7-tetrahydropyrazolo[1,5-a]pyrimidine-3-carboxamide;
Cis-5-(4-ethylphenyl)-7-isopropyl-N-(4-methylbenzyl)-4,5,6,7-tetrahydropyrazolo[1,5-a]pyrimidine-3-carboxamide;
Cis-N-(4-chlorobenzyl)-5-(4-ethylphenyl)-7-(trifluoromethyl)-4,5,6,7-tetrahydropyrazolo[1,5-a]pyrimidine-3-carboxamide;
Cis-7-(1,1-difluoroethyl)-5-(4-ethylphenyl)-N-(4-methylbenzyl)-4,5,6,7-tetrahydropyrazolo[1,5-a]pyrimidine-3-carboxamide;
Cis-5-(4-ethylphenyl)-N-((5-fluoropyridin-2-yl)methyl)-7-(trifluoromethyl)-4,5,6,7-tetrahydropyrazolo[1,5-a]pyrimidine-3-carboxamide;
Cis-N-((3,5-difluoropyridin-2-yl)methyl)-5-(4-ethylphenyl)-7-(trifluoromethyl)-4,5,6,7-tetrahydropyrazolo[1,5-a]pyrimidine-3-carboxamide;
Cis-5-(4-ethylphenyl)-7-isopropyl-N-(4-methoxybenzyl)-4,5,6,7-tetrahydropyrazolo[1,5-a]pyrimidine-3-carboxamide;
Cis-N-((1,5-dimethyl-1H-pyrrol-2-yl)methyl)-5-(4-ethylphenyl)-7-(trifluoromethyl)-4,5,6,7-tetrahydropyrazolo[1,5-a]pyrimidine-3-carboxamide;
Cis-N-(2-chlorobenzyl)-5-(4-ethylphenyl)-7-(trifluoromethyl)-4,5,6,7-tetrahydropyrazolo[1,5-a]pyrimidine-3-carboxamide;
Cis-5-(4-ethylphenyl)-N-((6-methoxypyridin-3-yl)methyl)-7-(trifluoromethyl)-4,5,6,7-tetrahydropyrazolo[1,5-a]pyrimidine-3-carboxamide;
Cis-N-((5-chloropyridin-2-yl)methyl)-5-(4-ethylphenyl)-7-(trifluoromethyl)-4,5,6,7-tetrahydropyrazolo[1,5-a]pyrimidine-3-carboxamide;
Cis-N-((6-chloropyridin-3-yl)methyl)-5-(4-ethylphenyl)-7-(trifluoromethyl)-4,5,6,7-tetrahydropyrazolo[1,5-a]pyrimidine-3-carboxamide;
Cis-5-(4-ethylphenyl)-N-((5-fluoro-6-methylpyridin-2-yl)methyl)-7-(trifluoromethyl)-4,5,6,7-tetrahydropyrazolo[1,5-a]pyrimidine-3-carboxamide; and
Cis-5-(4-methoxyphenyl)-N-(4-methylbenzyl)-7-(trifluoromethyl)-4,5,6,7-tetrahydropyrazolo[1,5-a]pyrimidine-3-carboxamide.

The invention also provides a pharmaceutically acceptable salt of a compound selected from the above list.

In another aspect, the invention provides any one of the compounds of Examples 1-41 as a single enantiomer, for example in greater than 95% e.e.

Reference hereinbelow to “Enantiomer a” means the enantiomer having the shorter retention time when the racemic mixture of the compound is separated into its enantiomers a and b, using semi-preparative high performance liquid chromatography (HPLC) under the following conditions: Column: Chiralpack IC, 250×20 mm, temperature 25° C., mobile phase: hexane/ethanol 90/10, Flow rate: 18 ml/minute, detection wavelength: 254 nm, and injection of 100 mg.

In another aspect the invention provides a compound of Formula (IC) which is selected from:
(5R,7S)-5-(4-ethylphenyl)-N-(4-methoxybenzyl)-7-(trifluoromethyl)-4,5,6,7-tetrahydropyrazolo[1,5-a]pyrimidine-3-carboxamide; (Enantiomer a of
cis-5-(4-ethylphenyl)-N-(4-methoxybenzyl)-7-(trifluoromethyl)-4,5,6,7-tetrahydropyrazolo[1,5-a]pyrimidine-3-carboxamide); and
(5R,7S)-5-(4-ethylphenyl)-N-(2-furanylmethyl)-7-(trifluoromethyl)-4,5,6,7-tetrahydropyrazolo[1,5-a]pyrimidine-3-carboxamide; (Enantiomer a of cis-5-(4-ethylphenyl)-N-(2-furanylmethyl)-7-(trifluoromethyl)-4,5,6,7-tetrahydropyrazolo[1,5-a]pyrimidine-3-carboxamide).

In another aspect the invention provides a compound of Formula (IC) which is selected from:
(5R,7S)-5-(4-ethylphenyl)-N-(2-fluoro-4-methylbenzyl)-7-(trifluoromethyl)-4,5,6,7-tetrahydropyrazolo[1,5-a]pyrimidine-3-carboxamide;
(5R,7S)-5-(4-ethylphenyl)-N-(4-fluorobenzyl)-7-(trifluoromethyl)-4,5,6,7-tetrahydropyrazolo[1,5-a]pyrimidine-3-carboxamide;
(5R,7S)-5-(4-ethylphenyl)-N-[(4-methylphenyl)methyl]-7-(trifluoromethyl)-4,5,6,7-tetrahydropyrazolo[1,5-a]pyrimidine-3-carboxamide;
(5R,7S)-5-(4-ethylphenyl)-N-(3-fluorobenzyl)-7-(trifluoromethyl)-4,5,6,7-tetrahydropyrazolo[1,5-a]pyrimidine-3-carboxamide;
(5R,7S)—N-(benzo[d][1,3]dioxol-5-ylmethyl)-5-(4-ethylphenyl)-7-(trifluoromethyl)-4,5,6,7-tetrahydropyrazolo[1,5-a]pyrimidine-3-carboxamide;
(5R,7S)-5-(4-ethylphenyl)-N-((5-methylfuran-2-yl)methyl)-7-(trifluoromethyl)-4,5,6,7-tetrahydropyrazolo[1,5-a]pyrimidine-3-carboxamide;
(5R,7S)—N-((3,5-difluoropyridin-2-yl)methyl)-5-(4-ethylphenyl)-7-(trifluoromethyl)-4,5,6,7-tetrahydropyrazolo[1,5-a]pyrimidine-3-carboxamide;
(5R,7R)-5-(4-ethylphenyl)-N-(4-methoxybenzyl)-7-methyl-4,5,6,7-tetrahydropyrazolo[1,5-a]pyrimidine-3-carboxamide;
(5R,7S)-5-(4-ethylphenyl)-N-((5-methylpyridin-2-yl)methyl)-7-(trifluoromethyl)-4,5,6,7-tetrahydropyrazolo[1,5-a]pyrimidine-3-carboxamide;
(5R,7S)-5-(4-ethylphenyl)-N-((6-methylpyridin-3-yl)methyl)-7-(trifluoromethyl)-4,5,6,7-tetrahydropyrazolo[1,5-a]pyrimidine-3-carboxamide;
(5R,7S)-5-(4-ethylphenyl)-N-((5-fluoro-6-methylpyridin-2-yl)methyl)-7-(trifluoromethyl)-4,5,6,7-tetrahydropyrazolo[1,5-a]pyrimidine-3-carboxamide;
(5R,7S)-5-(4-ethylphenyl)-N-((5-fluoropyridin-2-yl)methyl)-7-(trifluoromethyl)-4,5,6,7-tetrahydropyrazolo[1,5-a]pyrimidine-3-carboxamide;
(5R,7S)-5-(4-ethylphenyl)-N-((3-fluoro-5-methylpyridin-2-yl)methyl)-7-(trifluoromethyl)-4,5,6,7-tetrahydropyrazolo[1,5-a]pyrimidine-3-carboxamide; and
(5R,7S)-5-(4-ethylphenyl)-N-((3-fluoro-5-methylpyridin-2-yl)methyl)-7-(trifluoromethyl)-4,5,6,7-tetrahydropyrazolo[1,5-a]pyrimidine-3-carboxamide.

In another aspect the invention provides a compound of Formula (IC) which is selected from:
(5R,7S)-5-(4-ethylphenyl)-N-(3-methylbenzyl)-7-(trifluoromethyl)-4,5,6,7-tetrahydropyrazolo[1,5-a]pyrimidine-3-carboxamide;
(5R,7S)-7-(difluoromethyl)-5-(4-ethylphenyl)-N-(4-methoxybenzyl)-4,5,6,7-tetrahydropyrazolo[1,5-a]pyrimidine-3-carboxamide;
N-(3,4-difluorobenzyl)-5-(4-ethylphenyl)-7-(trifluoromethyl)-4,5,6,7-tetrahydropyrazolo[1,5-a]pyrimidine-3-carboxamide;
(5R,7S)—N-(3,4-difluorobenzyl)-5-(p-tolyl)-7-(trifluoromethyl)-4,5,6,7-tetrahydropyrazolo[1,5-a]pyrimidine-3-carboxamide;
(5S,7R)—N-(benzo[d][1,3]dioxol-5-ylmethyl)-7-(difluoromethyl)-5-(4-ethylphenyl)-4,5,6,7-tetrahydropyrazolo[1,5-a]pyrimidine-3-carboxamide;
(5R,7S)-5-(4-ethylphenyl)-N-(3-fluoro-4-methylbenzyl)-7-(trifluoromethyl)-4,5,6,7-tetrahydropyrazolo[1,5-a]pyrimidine-3-carboxamide;
(5R,7S)—N-(cyclohexylmethyl)-5-(4-ethylphenyl)-7-(trifluoromethyl)-4,5,6,7-tetrahydropyrazolo[1,5-a]pyrimidine-3-carboxamide;
(5R,7S)—N-(benzo[d][1,3]dioxol-5-ylmethyl)-5-(4-bromophenyl)-7-(trifluoromethyl)-4,5,6,7-tetrahydropyrazolo[1,5-a]pyrimidine-3-carboxamide;
(5R,7S)-5-(4-ethylphenyl)-N-((5-methylthiophen-2-yl)methyl)-7-(trifluoromethyl)-4,5,6,7-tetrahydropyrazolo[1,5-a]pyrimidine-3-carboxamide;
(5R,7S)-5-(4-ethylphenyl)-N-(4-fluoro-3-methylbenzyl)-7-(trifluoromethyl)-4,5,6,7-tetrahydropyrazolo[1,5-a]pyrimidine-3-carboxamide;
(5R,7S)-7-(tert-butyl)-5-(4-ethylphenyl)-N-(4-methoxybenzyl)-4,5,6,7-tetrahydropyrazolo[1,5-a]pyrimidine-3-carboxamide;
(5R,7S)-5-(4-ethylphenyl)-7-(trifluoromethyl)-N-(4-(trifluoromethyl)benzyl)-4,5,6,7-tetrahydropyrazolo[1,5-a]pyrimidine-3-carboxamide;
(5R,7S)-5-(4-ethylphenyl)-N-((6-methoxypyridin-3-yl)methyl)-7-(trifluoromethyl)-4,5,6,7-tetrahydropyrazolo[1,5-a]pyrimidine-3-carboxamide;
(5R,7S)—N-((6-chloropyridin-3-yl)methyl)-5-(4-ethylphenyl)-7-(trifluoromethyl)-4,5,6,7-tetrahydropyrazolo[1,5-a]pyrimidine-3-carboxamide; and
(5R,7S)-5-(4-ethylphenyl)-N-((5-fluoro-6-methoxypyridin-3-yl)methyl)-7-(trifluoromethyl)-4,5,6,7-tetrahydropyrazolo[1,5-a]pyrimidine-3-carboxamide.

In one embodiment the compound of Formula (IC) is (5R,7S)-5-(4-ethylphenyl)-N-(4-methoxybenzyl)-7-(trifluoromethyl)-4,5,6,7-tetrahydropyrazolo[1,5-a]pyrimidine-3-carboxamide.

In one embodiment the compound of Formula (IC) is (5R,7S)N-(benzo[d][1,3]dioxol-5-ylmethyl)-5-(4-ethylphenyl)-7-(trifluoromethyl)-4,5,6,7-tetrahydropyrazolo[1,5-a]pyrimidine-3-carboxamide.

The invention also provides a pharmaceutically acceptable salt of a compound of Formula (IC) selected from the above list.

### TERMS AND DEFINITIONS

The term “C₁₋₄alkyl” as used herein refers to a straight or branched chain alkyl group having 1 to 4 carbon atoms. Examples of C₁₋₄alkyl groups include methyl (Me), ethyl (Et), propyl (Pr) (for example n-propyl, iso-propyl), butyl (Bu) (for example n-butyl, sec-butyl, iso-butyl, tert-butyl (t-Bu)).

The term “compounds of the invention” as used herein means a compound of Formula (I) or a pharmaceutically acceptable salt thereof. The term “a compound of the invention” means any one of the compounds of the invention as defined above.

Furthermore, it will be understood that phrases such as “a compound of Formula (I) or a pharmaceutically acceptable salt thereof” or “compounds of the invention” are intended to encompass the compound of Formula (I), a pharmaceutically acceptable salt or solvate of the compound of Formula (I), or any pharmaceutically acceptable combination of these. Thus by way of non-limiting example used here for illustrative purpose, “a compound of Formula (I) or a pharmaceutically acceptable salt thereof” encompasses a pharmaceutically acceptable salt of a compound of Formula (I) which is present as a solvate, and this phrase also encompasses a mixture of a compound of Formula (I) and a salt of a compound of Formula (I).

It will be appreciated by those skilled in the art that whilst certain compounds of the invention can form pharmaceutically acceptable salts with an acid or a base, certain other compounds of the invention may not readily form such salts. It will be appreciated that all possible pharmaceutically acceptable salts of a compound of Formula (I) are contemplated to be within the scope of the present invention.

It will be appreciated that certain salts of the compounds according to Formula (I) may be prepared. Preferred salts are pharmaceutically acceptable salts. The compounds of the present invention may be administered as a pharmaceutically acceptable salt. Accordingly, the invention is further directed to pharmaceutically acceptable salts of the compounds according to Formula (I).

As used herein, the term “pharmaceutically acceptable salts” refers to salts that retain the desired biological activity of the subject compound and exhibit minimal undesired toxicological effects. For a review on suitable salts see Berge et al, J. Pharm. Sci., 1977, 66, 1-19. The term “pharmaceutically acceptable salts” includes both pharmaceutically acceptable acid addition salts and pharmaceutically acceptable base addition salts. These pharmaceutically acceptable salts may be prepared in situ during the final isolation and purification of the compound, or by separately reacting the purified compound in its free acid or free base form with a suitable base or acid, respectively. The salt may precipitate from solution and be collected by filtration or may be recovered by evaporation of the solvent.

Embodiments of compounds according to Formula (I) that contain a basic functional group may be capable of forming pharmaceutically acceptable acid addition salts by treatment with a suitable acid, preferably a strong acid. A pharmaceutically acceptable acid addition salt may be formed by reaction of a compound of Formula (I) with a suitable inorganic or organic acid, including, but not limited to: hydrobromic, hydroiodic, sulfuric, nitric, perchloric, p-toluenesulfonic and benzenesulfonic, acids, optionally in a suitable solvent such as an organic solvent, to give the acid addition salt which is usually isolated for example by crystallisation and filtration. Pharmaceutically acceptable acid addition salts include, but are not limited to: hydrobromide, hydroiodide, sulfate, bisulfate, nitrate, perchlorate, p-toluenesulfonate, and benzenesulfonate, salts. For Example, Examples 10, 11, 19 and 20 of the present invention may form such acid addition salts.

The invention includes within its scope all possible stoichiometric and non-stoichiometric forms of the salts of the compounds of Formula (I). In one embodiment of the invention there is provided a pharmaceutically acceptable salt of a compound of Formula (I).

It will be further appreciated that all crystalline forms, polymorphs and enantiomers of the compounds of the invention, or mixtures thereof, are contemplated to be within the scope of the present invention. Unless otherwise specified (for example when the absolute stereochemistry is shown), for compounds of the invention which possess at least one stereocentre, and which can therefore form enantiomers, the compound can contain a mixture of enantiomers, for example a 1:1 mixture of enantiomers, i.e. a racemic mixture of enantiomers. This mixture of enantiomers may be separated using conventional techniques such as chiral HPLC. For an isomer of compound of the invention for which the absolute stereochemistry is stated or which is otherwise described as a single enantiomer, said isomer of a compound of the invention has, in one embodiment, at least 80% e.e. In another embodiment, said isomer of a compound of the invention has at least 90% e.e., for example at least 95% e.e. In another embodiment said isomer of compound of the invention corresponds to at least 98% e.e, for example at least 99% e.e.

Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Since the compounds of Formula (I) are intended for use in pharmaceutical compositions it will readily be understood that in particular embodiments they are provided in substantially pure form, for example at least 60% pure, more suitably at least 75% pure and particularly at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and more particularly from 10 to 59% of a compound of Formula (I) or pharmaceutically acceptable salt and/or solvate thereof.

### Compound Preparation

The general procedures used to synthesise the compounds of Formula (I) are summarised in reaction Scheme 1 and are illustrated in the Examples.

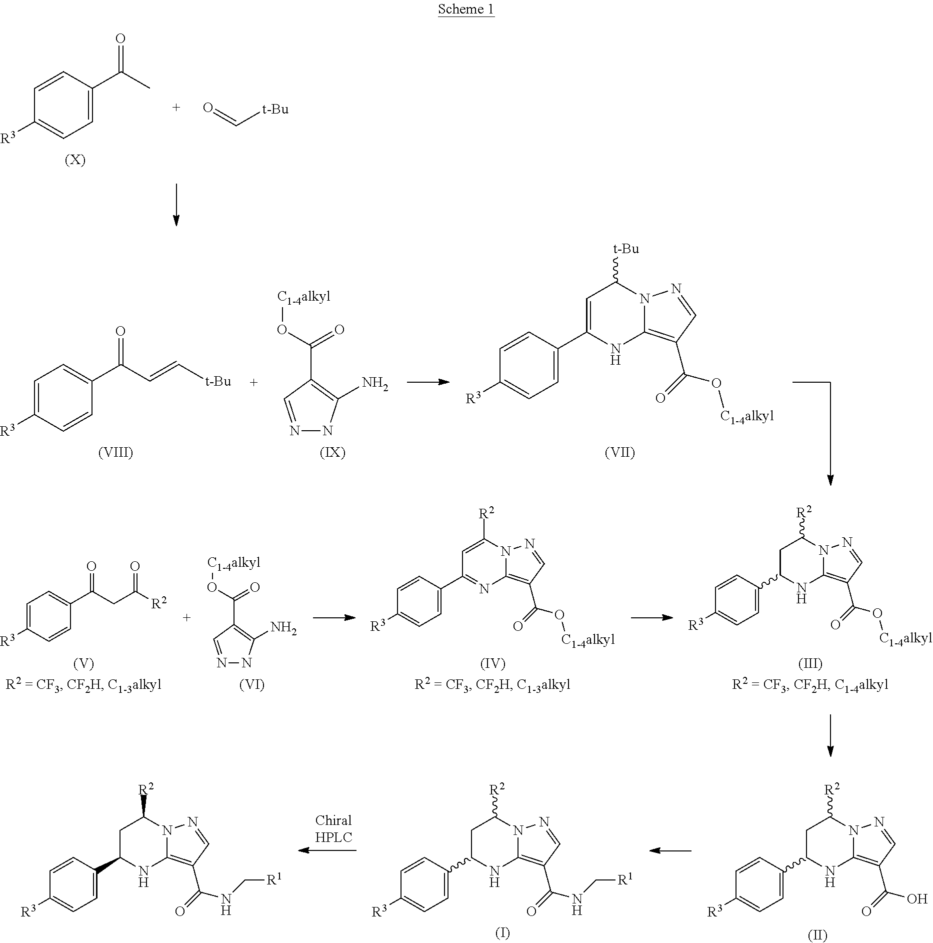

Compounds of Formula (I) may be prepared from compounds of Formula (II), wherein R² and R³ are as described herein for Formula (I), by reaction of compounds (II) with an amine R¹CH₂NH₂, wherein R¹ is as described herein for Formula (I), or with a salt of such an amine (e.g. the hydrochloride salt), in the presence of a suitable base such as N,N-diisopropylethylamine or triethylamine and a suitable coupling reagent, such as HATU, HOBt or EDC, in the presence of a suitable solvent, such as DMF or DCM, at elevated temperature, for example at 60° C.

Compounds of Formula (II) may be prepared from compounds of Formula (III), wherein R² and R³ are as described herein for Formula (I), by reaction of compounds (III) with i) a suitable base, such as KOH, NaOH or LiOH, in the presence of a suitable solvent, such as EtOH, MeOH or THF at elevated temperature, for example at 60° C.

Compounds of Formula (III), wherein R² represents CF₃, C₁₋₃alkyl or CHF₂, may be prepared from compounds of Formula (IV), wherein R² represents CF₃, C₁₋₃alkyl or CHF₂ and R³ is as described herein for Formula (I), either i) by reaction of compounds (IV) with a reducing agent, such as sodium borohydride or sodium triacetoxyborohydride, in the presence of a suitable solvent, such as MeOH or EtOH; or ii) by hydrogenation of compounds (IV) under standard conditions.

Compounds of Formula (IV) may be prepared by a reaction between commercially available compounds of Formula (V), wherein R² represents CF₃, C₁₋₃alkyl or CHF₂ and R³ is as described herein for Formula (I), and commercially available compounds of Formula (VI), wherein C₁₋₄alkyl is ethyl, in the presence of a suitable acid such as AcOH or TFA at elevated temperature, such as at reflux.

Compounds of Formula (III), wherein R² represents t-Bu, may be prepared from compounds of Formula (VII), wherein, R³ is as described herein for Formula (I), either i) by reaction of compounds (VII) with a reducing agent, such as sodium borohydride or sodium triacetoxyborohydride, in the presence of a suitable solvent, such as MeOH or EtOH; or ii) by hydrogenation of compounds (VII) under standard conditions.

Compounds of Formula (VII) may be prepared by a reaction between compounds of Formula (VIII), wherein R³ is as described herein for Formula (I), and commercially available compounds of Formula (IX), wherein C₁₋₄alkyl is ethyl, by treatment with a suitable acid, such as TFA, in a suitable solvent, such as 2-(methyloxy)ethanol, at reduced temperature, such as between 0° C. and +5° C., followed by elevated temperature, such as reflux.

Compounds of Formula (VIII) may be prepared by a reaction between commercially available compounds of Formula (X), wherein R³ is as described herein for Formula (I), and pivalaldehyde in the presence of a suitable base, such as NaOH, KOH, triethylamine or sodium carbonate, in the presence of a suitable solvent, such as MeOH, EtOH or THF.

Amines of formula R¹CH₂NH₂ are commercially available or may be prepared by methods well known in the art.

Compounds of Formula (I) where R² is a C₁₋₄ alkyl group (e.g methy, isopropyl or t-butyl) may also be prepared according to the following scheme 1A, which is shown with reference to the preparation of compounds where R² is iso-propyl:

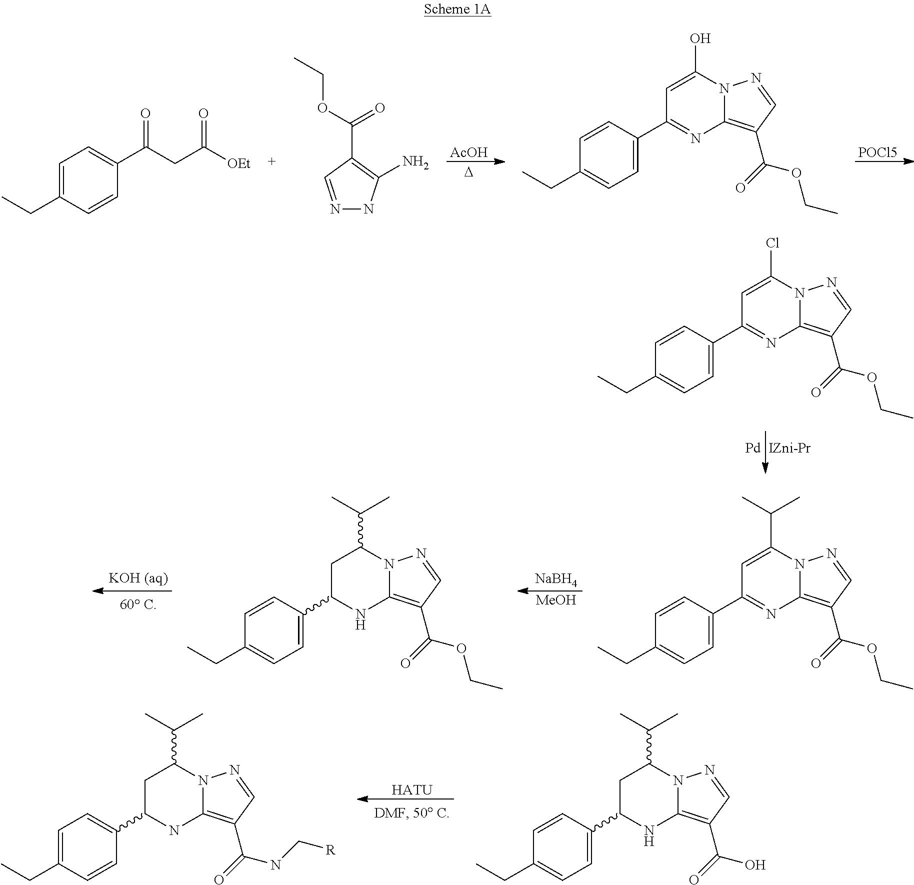

Compounds of formula (I) prepared according to Scheme 1 or Scheme 1A are initially obtained as mixtures of enantiomers. The enantiomers may be separated by chiral HPLC. Alternatively compounds of the invention may be prepared in enantiomeric form by using a chiral intermediate, for example using the method of Scheme 2 below.

The absolute configuration of enantiomers according to the invention may be determined by ab initio vibrational circular dichroism (VCD) with a level of reliability >99%. In accordance with standard practice the absolute configuration for related structures may be assigned by analogy.

Enantiomers of Formula (I) wherein R² represents CF₃ and R³ represents ethyl, may be prepared via a chiral intermediate, according to reaction scheme 2:

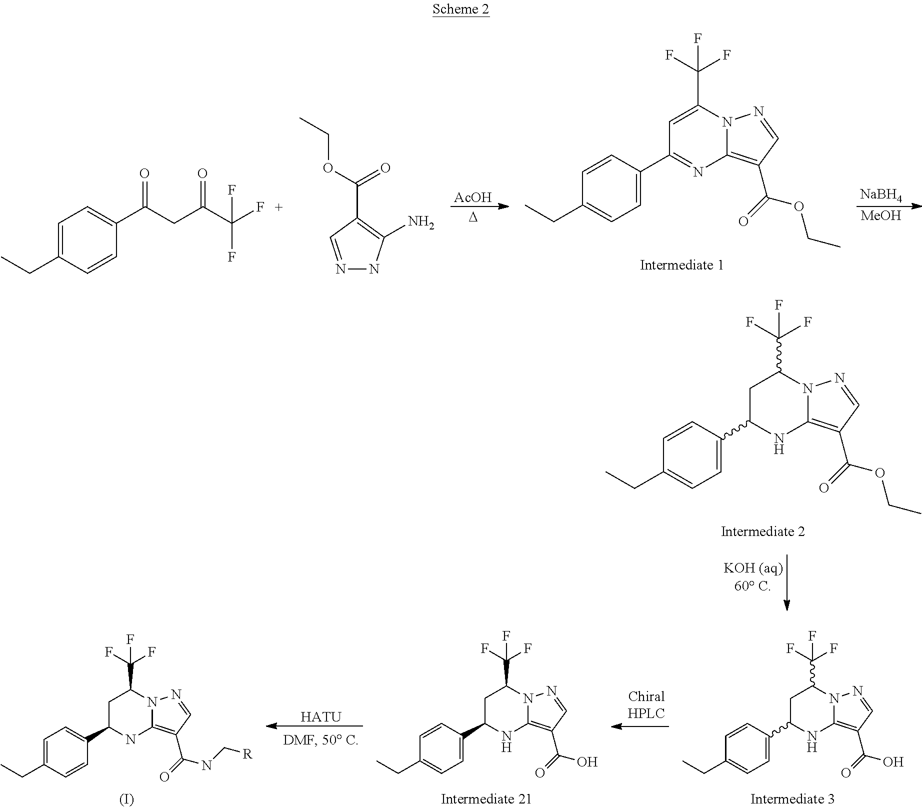

Those skilled in the art will appreciate that in the preparation of the compound of Formula (I), it may be necessary and/or desirable to protect one or more sensitive groups in the molecule or the appropriate intermediate to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P. J. Kocienski (Georg Thieme Verlag 1994).

It will be readily apparent to those skilled in the art that other compounds of Formula (I) may be prepared using methods analogous to those outlined above, or by reference to the experimental procedures detailed in the Examples provided herein. Further details for the preparation of compounds of Formula (I) are found in the Examples.

### Compositions and Formulations

The compounds of the invention may be formulated for administration in any convenient way for use in human or veterinary medicine, by analogy with formulation of antibacterials, or formulation of other antitubercular agents.

The compounds of the invention will normally, but not necessarily, be formulated into pharmaceutical compositions prior to administration to a patient. In one aspect, the invention is directed to a pharmaceutical composition comprising a compound of Formula (I), or a pharmaceutically acceptable salt thereof. In another aspect the invention is directed to a pharmaceutical composition comprising a compound of Formula (I), or a pharmaceutically acceptable salt thereof, and one or more pharmaceutically acceptable carriers, excipients or diluents. The carrier, excipient or diluent must be “acceptable” in the sense of being compatible with the other ingredients of the formulation and not deleterious to the recipient thereof.

A therapeutically effective amount of the compound of the present invention can be determined by methods known in the art. The therapeutically effective quantities will depend on the age and on the general physiological condition of the subject, the route of administration and the pharmaceutical formulation used. The therapeutic doses will generally be between about 1 and 2000 mg/day, for example between about 500 and 2000 mg/day. The daily dose as employed for acute or chronic human treatment will range from 0.01 to 250 mg/kg body weight, which may be administered in one or two daily doses, for example, depending on the route of administration and the condition of the subject. When the composition comprises dosage units, each unit will contain 1 mg to 2 g of active ingredient.

The present invention is further related to a pharmaceutical composition for the treatment of tuberculosis, comprising the compound of Formula (I) or a pharmaceutically acceptable salt thereof.

The present invention is even further related to a pharmaceutical composition comprising a) 500 to 2000 mg of the compound of Formula (I) or a pharmaceutically acceptable salt thereof, and b) 0.1 to 2 g of one or more pharmaceutically acceptable excipients.

The pharmaceutical compositions of the invention include those in a form adapted for oral use in mammals including humans.

The pharmaceutical compositions of the invention include those in a form adapted for oral use and may be used for the treatment of tuberculosis in mammals including humans.

The composition may be formulated for administration by any convenient route. For the treatment of tuberculosis, the compositions may be in the form of tablets, capsules, powders, granules, lozenges, aerosols or liquid preparations, for oral use.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

In one aspect of the invention, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use.

The compound of Formula (I), or a pharmaceutically acceptable salt thereof, may be the sole therapeutic agent in the compositions of the invention, or it may be present in the formulation in combination with one or more additional therapeutic agents.

The invention thus provides in a further aspect, a combination comprising a compound of Formula (I), or a pharmaceutically acceptable salt thereof together with one or more additional therapeutic agents. Examples of such one or more additional therapeutic agents are anti-tuberculosis agents including, but not limited to, amikacin, aminosalicylic acid, capreomycin, cycloserine, ethambutol, ethionamide, isoniazid, kanamycin, pyrazinamide, rifamycins (such as rifampin, rifapentine and rifabutin), streptomycin, clarithromycin, azithromycin, oxazolidinones and fluoroquinolones (such as ofloxacin, ciprofloxacin, moxifloxacin and gatifloxacin). Such chemotherapy is determined by the judgment of the treating physician using preferred drug combinations. “First-line” chemotherapeutic agents used to treat a Mycobacterium tuberculosis infection that is not drug resistant include isoniazid, rifampin, ethambutol, streptomycin and pyrazinamide. “Second-line” chemotherapeutic agents used to treat a Mycobacterium tuberculosis infection that has demonstrated drug resistance to one or more “first-line” drugs include ofloxacin, ciprofloxacin, ethionamide, aminosalicylic acid, cycloserine, amikacin, kanamycin and capreomycin. In addition to the aforementioned, there is a number of new anti-tuberculosis therapeutic agents emerging from clinical studies that may also be employed as the one or more additional therapeutic agents in a combination with a compound of Formula (I), including, but not limited to, TMC-207, OPC-67683, PA-824, LL-3858 and SQ-109.

In another aspect, the invention provides a combination comprising a compound of Formula (I), or a pharmaceutically acceptable salt thereof together with one or more additional therapeutic agents, such as an anti-tuberculosis agent, especially isoniazid (INH), rifampin, pyrazinamide and ethambutol and/or an anti-bacterial agent or an anti-AIDS agent.

In a further aspect, the one or more additional therapeutic agent is, for example, an agent useful for the treatment of tuberculosis in a mammal, therapeutic vaccines, anti-bacterial agents, anti-viral agents; antibiotics and/or agents for the treatment of HIV/AIDS. Examples of such therapeutic agents include isoniazid (INH), ethambutol, rifampin, pirazinamide, streptomycin, capreomycin, ciprofloxacin and clofazimine.

In one aspect, the one or more additional therapeutic agent is a therapeutic vaccine. A compound of Formula (I), or a pharmaceutically acceptable salt thereof, may thus be administered in conjunction with vaccination against mycobacterial infection, in particular vaccination against Mycobacterium tuberculosis infection. Existing vaccines against mycobacterial infection include Bacillus Calmette Guerin (BCG). Vaccines currently under development for the treatment, prophylaxis or amelioration of mycobacterial infection include: modified BCG strains which recombinantly express additional antigens, cytokines and other agents intended to improve efficacy or safety; attenuated mycobacteria which express a portfolio of antigens more similar to Mycobacterium tuberculosis than BCG; and subunit vaccines. Subunit vaccines may be administered in the form of one or more individual protein antigens, or a fusion or fusions of multiple protein antigens, either of which may optionally be adjuvanted, or in the form of a polynucleotide encoding one or more individual protein antigens, or encoding a fusion or fusions of multiple protein antigens, such as where the polynucleotide is administered in an expression vector. Examples of subunit vaccines include, but are not limited to: M72, a fusion protein derived from the antigens Mtb32a and Mtb39; HyVac-1, a fusion protein derived from antigen 85b and ESAT-6; HyVac-4, a fusion protein derived from antigen 85b and Tb10.4; MVA85a, a modified vaccinia virus Ankara expressing antigen 85a; and Aeras-402, adenovirus 35 expressing a fusion protein derived from antigen 85a, antigen 85b and Tb10.4.

The compound of Formula (I), or a pharmaceutically acceptable salt thereof, may be either i) administered to an individual who has previously been vaccinated against mycobacterial infection; ii) administered to an individual who is subsequently vaccinated against mycobacterial infection; or iii) may be co-administered with a vaccine against mycobacterial infection, either by administering the compound of the invention and the vaccine together in the same dosage form or co-administering the compound of the invention and the vaccine in separate dosage forms.

When a compound of Formula (I), or a pharmaceutically acceptable salt thereof is used in combination with one or more additional therapeutic agents, the dose of the compound or agent may differ from that when the compound or agent is used alone. Appropriate doses will be readily appreciated by those skilled in the art. It will be appreciated that the amount of a compound of the invention and the one or more additional therapeutic agents required for use in treatment will vary with the nature of the condition being treated and the age and the condition of the patient and will be ultimately at the discretion of the attendant physician or veterinarian.

The combinations may conveniently be presented for use in the form of a pharmaceutical formulation. In a further aspect of the present invention there is provided a pharmaceutical combination comprising a compound of Formula (I), or a pharmaceutically acceptable salt thereof, together with one or more additional therapeutic agents, and one or more pharmaceutically acceptable carriers, excipients or diluents. The individual components of such combinations may be administered either sequentially or simultaneously in separate or combined pharmaceutical formulations by any convenient route.

When administration is sequential, either the compound of the present invention or one or more additional therapeutic agent may be administered first. When administration is simultaneous, the combination may be administered either in the same or different pharmaceutical composition. When combined in the same formulation it will be appreciated that the compound and agents must be stable and compatible with each other and the other components of the formulation. When formulated separately they may be provided in any convenient formulation, conveniently in such manner as are known for such compounds in the art.

### ABBREVIATIONS

In describing the invention, chemical elements are identified in accordance with the Periodic Table of the Elements. Abbreviations and symbols utilized herein are in accordance with the common usage of such abbreviations and symbols by those skilled in the chemical arts. The following abbreviations are used herein:
ACN acetonitrile
AcOH acetic acid
CDCl₃ deuterated chloroform
DCM dichloromethane
DIPEA N,N-diisopropylethylamine
DMF dimethylformamide
DMSO-d₆ deuterated dimethylsulfoxide
EDC 1-ethyl-3-(3-dimethyllaminopropyl)carbodiimide
ES MS Electrospray mass spectrometry
Et Ethyl
EtOAc ethyl acetate
EtOH ethanol
Ex Example
h hours
HATU N,N,N′,N′-Tetramethyl-O-(7-azabenzotriazol-1-yl)uronium hexafluorophosphate
HPLC high performance liquid chromatography
HOBt hydroxybenzotriazole
KOH potassium hydroxide
LCMS Liquid chromatography mass spectroscopy
M Molar
Me methyl
MeOH methanol
MeONa sodium methoxide
MgSO₄ magnesium sulphate
min minutes
MS mass spectrum
NaHCO₃ sodium bicarbonate
Na₂SO₄ sodium sulphate
NH₄HCO₃ ammonium bicarbonate
NMR Nuclear Magnetic Resonance spectroscopy
TFA trifluoroacetic acid
THF tetrahydrofuran
t-Bu tert-butyl
i-Pr iso-propyl

### Examples

The following Examples illustrate the invention. These Examples are not intended to limit the scope of the invention, but rather to provide guidance to the skilled artisan to prepare and use the compounds, compositions, and methods of the invention. While particular embodiments of the invention are described, the skilled artisan will appreciate that various changes and modifications can be made. References to preparations carried out in a similar manner to, or by the general method of, other preparations, may encompass variations in routine parameters such as time, temperature, workup conditions, minor changes in reagent amounts etc.

Proton nuclear magnetic resonance (¹H NMR) spectra were recorded, and chemical shifts are reported in parts per million (δ) downfield from the internal standard tetramethylsilane (TMS). Abbreviations for NMR data are as follows: s=singlet, d=doublet, t=triplet, q=quartet, m=multiplet, dd=doublet of doublets, dt=doublet of triplets, app=apparent, br=broad. Mass spectra were obtained using electrospray (ES) ionization techniques. All temperatures are reported in degrees centigrade.

Reactions involving metal hydrides including sodium borohydride and sodium triacetoxyborohydride are carried out under argon or nitrogen unless otherwise specified.

In the following Intermediates and Examples, where the relative stereochemistry of the compound has been identified, this is indicated both in the name of the compound (for example “cis”).

In certain of the following Intermediates and Examples, starting materials are identified by reference to other Intermediate or Example numbers. This does not signify that the actual material from any particular Intermediate or Example was necessarily used in a subsequent step exemplified herein, but is used as a short-hand means of denoting the relevant compound name.

### Intermediates

### Intermediate 1: Ethyl 5-(4-ethylphenyl)-7-(trifluoromethyl)pyrazolo[1,5-a]pyrimidine-3-carboxylate

A mixture of ethyl 3-aminopyrazole-4-carboxylate (ALDRICH, 0.635 g, 4.090 mmol) and 1-(4-ethyl-phenyl)-4,4,4-trifluoro-butane-1,3-dione (ARTCHEM, 1 g, 4.090 mmol) in AcOH (10 mL) was heated at reflux for 6 h. After cooling to rt the reaction mixture was poured onto ice (60 g). The solid formed was filtered off, triturated with hexane and dried to afford a pale yellow solid (950 mg, 2.61 mmol, 64%). ¹H NMR (400 MHz, CDCl₃) δ ppm: 8.66 (s, 1H), 8.18 (d, 2H), 7.77 (s, 1H), 7.39 (d, 2H), 4.47 (q, 2H), 2.76 (q, 2H), 1.47 (t, 3H), 1.30 (t, 3H). [ES+MS] m/z 364 (M+H)⁺.

### Intermediate 2: Cis-ethyl 5-(4-ethylphenyl)-7-(trifluoromethyl)-4,5,6,7-tetrahydropyrazolo[1,5-a]pyrimidine-3-carboxylate

To a suspension of

Intermediate 1 (480 mg, 1.321 mmol) in dry MeOH (11 mL) was added sodium borohydride (ALDRICH, 175 mg, 4.620 mmol) in 4 portions at rt. The reaction was stirred for 5 h and checked by LCMS. Completion of the reaction was observed. The reaction was quenched with saturated citric acid solution, concentrated in vacuo and extracted with EtOAc (3×10 mL). The organic solution was washed with aqueous NaHCO₃ solution, water and brine, dried over anhydrous MgSO₄ and concentrated to afford a pale yellow solid (452 mg, 1.23 mmol, 93%). ¹H NMR (400 MHz, CDCl₃) δ ppm: 7.74 (s, 1H), 7.36 (d, 2H), 7.25 (d, 2H), 6.12 (bs, 1H), 4.87-4.81 (m, 1H), 4.55 (dd, 1H), 4.28-4.22 (m, 2H), 2.68 (q, 2H), 2.57-2.51 (m, 1H), 2.41-2.32 (m, 1H), 1.32 (t, 3H), 1.26 (t, 3H). [ES+MS] m/z 368 (M+H)⁺.

### Intermediate 3: Cis-5-(4-ethylphenyl)-7-(trifluoromethyl)-4,5,6,7-tetrahydropyrazolo[1,5-a]pyrimidine-3-carboxylic acid

To a solution of Intermediate 2 (1 g, 2.720 mmol) in ethanol (5 mL) was added a 1.5 M aqueous solution of KOH (3.5 mL, 5.250 mmol) and the reaction was stirred 12 h at 60° C. The reaction was concentrated in vacuo to remove the organic solvent and a 0.1M citric acid solution was them added to acid pH. The precipitated solid was collected by filtration, washed with water and dried to afford a pale yellow solid (850 mg, 2.50 mmol, 92%). ¹H NMR (400 MHz, DMSO-d₆) δ ppm: 12.08 (bs, 1H), 7.61 (s, 1H), 7.35 (d, 2H), 7.24 (d, 2H), 6.40 (bs, 1H), 5.30-5.25 (m, 1H), 4.64 (dd, 1H), 2.60 (q, 2H), 2.20-2.12 (m, 1H), 1.17 (t, 3H). [ES+MS] m/z 340 (M+H)⁺.

### Intermediate 4: Ethyl 5-(4-bromophenyl)-7-(trifluoromethyl)pyrazolo[1,5-a]pyrimidine-3-carboxylate

The title compound was prepared by a method analogous to that described for Intermediate 1. Ethyl 3-aminopyrazole-4-carboxylate (ALDRICH, 0.578 g, 3.730 mmol) and 1-(4-bromo-phenyl)-4,4,4-trifluoro-butane-1,3-dione (FLUOROCHEM, 1 g, 3.390 mmol) as starting reactants. The title compound (1.2 g, 2.90 mmol, 85%) was obtained as a pale yellow solid. ¹H NMR (400 MHz, CDCl₃) δ ppm: 8.68 (s, 1H), 8.14 (d, 2H), 7.76 (s, 1H), 7.71 (d, 2H), 4.47 (q, 2H), 1.47 (t, 3H). [ES+MS] m/z 414 (M+H)⁺.

### Intermediate 5: Cis-ethyl 5-(4-bromophenyl)-7-(trifluoromethyl)-4,5,6,7-tetrahydropyrazolo[1,5-a]pyrimidine-3-carboxylate

The title compound was prepared by a method analogous to that described for Intermediate 2. Intermediate 4 (1.0 g, 2.414 mmol) and sodium borohydride (ALDRICH, 320 mg, 8.450 mmol) as starting reactants. The title compound (900 mg, 2.152 mmol, 89%) was obtained as a pale yellow solid. ¹H NMR (400 MHz, CDCl₃) δ ppm: 7.74 (s, 1H), 7.56 (d, 2H), 7.34 (d, 2H), 6.13 (bs, 1H), 4.87-4.82 (m, 1H), 4.55 (dd, 1H), 4.29-4.23 (m, 2H), 2.56-2.50 (m, 1H), 2.39-2.38 (m, 1H), 1.33 (t, 3H). [ES+MS] m/z 418 (M+H)⁺.

### Intermediate 6: Cis-5-(4-bromophenyl)-7-(trifluoromethyl)-4,5,6,7-tetrahydropyrazolo[1,5-a]pyrimidine-3-carboxylic acid

The title compound was prepared by a method analogous to that described for Intermediate 3. Intermediate 5 (900 mg, 2.152 mmol) and potassium hydroxide (3.5 mL, 5.250 mmol) as starting reactants. The title compound (700 mg, 1.794 mmol, 83%) was obtained as a pale yellow solid. ¹H NMR (400 MHz, DMSO-d₆) δ ppm: 12.09 (bs, 1H), 7.61 (s, 1H), 7.58 (d, 2H), 7.40 (d, 2H), 6.68 (bs, 1H), 5.28-5.20 (m, 1H), 4.67 (dd, 1H), 2.24-2.15 (m, 1H). [ES+MS] m/z 390 (M+H)⁺.

### Intermediate 7: Ethyl 7-(4-ethylphenyl)-7-methylpyrazolo[1,5-a]pyrimidine-3-carboxylate

The title compound was prepared by a method analogous to that described for Intermediate 1. Ethyl 3-aminopyrazole-4-carboxylate (ALDRICH, 0.816 g, 5.260 mmol) and 1-(4-ethylphenyl)-1,3-butanedione (PARAGOS, 1 g, 5.260 mmol) as starting reactants. The title compound (220 mg, 0.711 mmol, 13%) was obtained as a white solid. ¹H NMR (400 MHz, CDCl₃) δ ppm: 8.57 (s, 1H), 8.16 (d, 2H), 7.35 (d, 2H), 7.33 (s, 1H), 4.46 (q, 2H), 2.88 (s, 3H), 2.73 (q, 2H), 1.47 (t, 3H), 1.29 (t, 3H). [ES+MS] m/z 310 (M+H)⁺.

### Intermediate 8: Cis-ethyl 5-(4-ethylphenyl)-7-methyl-4,5,6,7-tetrahydropyrazolo[1,5-a]pyrimidine-3-carboxylate

The title compound was prepared by a method analogous to that described for Intermediate 2. Intermediate 7 (150 mg, 0.485 mmol) and sodium borohydride (ALDRICH, 64.2 mg, 1.697 mmol) as starting reactants. The title compound (125 mg, 0.399 mmol, 82%) was obtained as a pale yellow oil. ¹H NMR (400 MHz, CDCl₃) δ ppm: 7.65 (s, 1H), 7.33 (d, 2H), 7.22 (d, 2H), 5.91 (bs, 1H), 4.55 (dd, 1H), 4.35-4.28 (m, 1H), 4.24 (q, 2H), 2.67 (q, 2H), 2.34-2.29 (m, 1H), 2.04-1.95 (m, 1H), 1.60 (d, 3H), 1.31 (t, 3H), 1.25 (t, 3H).

### Intermediate 9: Cis-5-(4-ethylphenyl)-7-methyl-4,5,6,7-tetrahydropyrazolo[1,5-a]pyrimidine-3-carboxylic acid

The title compound was prepared by a method analogous to that described for Intermediate 3. Intermediate 8 (125 mg, 0.407 mmol) and potassium hydroxide (0.968 mL, 1.452 mmol) as starting reactants. The title compound (100 mg, 0.350 mmol, 84%) was obtained as a white solid. ¹H NMR (400 MHz, DMSO-d₆) δ ppm: 11.89 (bs, 1H), 7.47 (s, 1H), 7.33 (d, 2H), 7.22 (d, 2H), 6.04 (bs, 1H), 4.56 (dd, 1H), 4.28-4.23 (m, 1H), 2.24-2.15 (m, 1H), 2.59 (q, 2H), 2.31-2.27 (m, 1H), 1.90-1.81 (m, 1H), 1.42 (d, 3H), 1.17 (t, 3H).

### Intermediate 10: 4,4,4-trifluoro-1-(p-tolyl)butane-1,3-dione

To a solution of MeONa prepared from sodium (1.919 g, 83.0 mmol) and methanol (60 mL) was added ethyl 2,2,2-trifluoroacetate (ALDRICH, 8.19 mL, 54.8 mmol) and the reaction was stirred at rt 30 min. Then 1-(p-tolyl)ethanone (ALDRICH, 6.97 mL, 52.2 mmol) was added, the reaction mixture was heated at 60° C. overnight. The reaction was checked by LCMS and the end of the reaction was observed. The reaction mixture was concentrated under vacuum and partitioned between sodium carbonate (10%) 25 mL and DCM (25 mL), the organic layer was separated, dried over Na₂SO₄, filtered and concentrated under vacuum to afford a brown solid (10.2 g, 44.3 mmol, 85%). ¹H NMR (400 MHz, CDCl₃) δ ppm: 7.85 (d, 2H), 7.30 (d, 2H), 6.56 (s, 1H), 2.45 (s, 3H).

### Intermediate 11: Ethyl 5-(p-tolyl)-7-(trifluoromethyl)pyrazolo[1,5-a]pyrimidine-3-carboxylate

The title compound was prepared by a method analogous to that described for Intermediate 1. Ethyl 3-aminopyrazole-4-carboxylate (ALDRICH, 1.541 g, 9.930 mmol) and Intermediate 10 (2.286 g, 9.930 mmol) as starting reactants. The title compound (3.06 g, 8.760 mmol, 88%) was obtained as a pale yellow solid. ¹H NMR (400 MHz, CDCl₃) δ ppm: 8.66 (s, 1H), 8.16 (d, 2H), 7.77 (s, 1H), 7.38 (d, 2H), 4.47 (q, 2H), 2.47 (s, 3H), 1.47 (t, 3H). [ES+MS] m/z 350 (M+H)⁺.

### Intermediate 12: Cis-ethyl 5-(p-tolyl)-7-(trifluoromethyl)-4,5,6,7-tetrahydropyrazolo[1,5-a]pyrimidine-3-carboxylate

The title compound was prepared by a method analogous to that described for Intermediate 2. Intermediate 11 (3.02 g, 8.650 mmol) and sodium borohydride (ALDRICH, 2.12 g, 56.240 mmol) as starting reactants. The title compound (3.03 g, 8.580 mmol, 99%) was obtained as an orange oil. ¹H NMR (400 MHz, CDCl₃) δ ppm: 7.74 (s, 1H), 7.33 (d, 2H), 7.23 (d, 2H), 6.11 (bs, 1H), 4.88-4.80 (m, 1H), 4.56-4.53 (dd, 1H), 4.25 (q, 2H), 2.56-2.50 (m, 1H), 2.40-2.31 (m, 4H), 1.33 (t, 3H). [ES+MS] m/z 354 (M+H)⁺.

### Intermediate 13: Cis-5-(p-tolyl)-7-(trifluoromethyl)-4,5,6,7-tetrahydropyrazolo[1,5-a]pyrimidine-3-carboxylic acid

The title compound was prepared by a method analogous to that described for Intermediate 3. Intermediate 12 (3.09 g, 8.750 mmol) and potassium hydroxide (24 mL, 36.0 mmol) as starting reactants. The title compound (2.684 g, 8.250 mmol, 94%) was obtained as a white solid. ¹H NMR (400 MHz, DMSO-d₆) δ ppm: 12.07 (bs, 1H), 7.61 (s, 1H), 7.33 (d, 2H), 7.20 (d, 2H), 6.40 (bs, 1H), 5.30-5.24 (m, 1H), 4.64-4.62 (dd, 1H), 2.30 (s, 3H), 2.21-2.12 (m, 1H). [ES+MS] m/z 326 (M+H)⁺.

### Intermediate 14: Ethyl 7-(difluoromethyl)-5-(4-ethylphenyl)pyrazolo[1,5-a]pyrimidine-3-carboxylate

The title compound was prepared by a method analogous to that described for Intermediate 1. Ethyl 3-aminopyrazole-4-carboxylate (ALDRICH, 1.5 g, 9.670 mmol) and 1-(4-ethylphenyl)-4,4-difluorobutane-1,3-dione (ZELINSKY, 2.286 g, 10.110 mmol) as starting reactants. The title compound (2.4 g, 6.950 mmol, 69%) was obtained as a pale green solid. ¹H NMR (400 MHz, CDCl₃) δ ppm: 8.60 (s, 1H), 8.19 (d, 2H), 7.74 (s, 1H), 7.40-7.37 (m, 3H), 4.47 (q, 2H), 2.76 (q, 2H), 1.47 (t, 3H), 1.30 (t, 3H). [ES+MS] m/z 346 (M+H)⁺.

### Intermediate 15: Cis-ethyl 7-(difluoromethyl)-5-(4-ethylphenyl)-4,5,6,7-tetrahydropyrazolo[1,5-a]pyrimidine-3-carboxylate

The title compound was prepared by a method analogous to that described for Intermediate 2. Intermediate 14 (2 g, 5.790 mmol), sodium borohydride (ALDRICH, 0.767 g, 20.270 mmol) as starting reactants. The title compound (1.87 g, 5.350 mmol, 92%) was obtained as pale yellow solid in a mixture of cis/trans 85/15. ¹H NMR (400 MHz, CDCl₃) δ ppm: 7.68 (s, 1H), 7.36 (d, 2H), 7.25 (d, 2H), 6.70-6.41 (m, 1H), 5.99 (bs, 1H), 4.58-4.54 (m, 2H), 4.27-4.22 (m, 2H), 2.68 (q, 2H), 2.42-2.38 (m, 2H), 1.32 (t, 3H), 1.26 (t, 3H). [ES+MS] m/z 350 (M+H)⁺.

### Intermediate 16: Cis-7-(difluoromethyl)-5-(4-ethylphenyl)-4,5,6,7-tetrahydropyrazolo[1,5-a]pyrimidine-3-carboxylic acid

The title compound was prepared by a method analogous to that described for Intermediate 3. Intermediate 15 (1.87 g, 5.350 mmol) and potassium hydroxide (3.5 mL, 5.250 mmol) as starting reactants. The title compound (1.47 g, 4.570 mmol, 85%) was obtained as a white solid in a mixture of cis/trans 85/15. ¹H NMR (400 MHz, DMSO-d₆) δ ppm: 7.56 (s, 1H), 7.35 (d, 2H), 7.24 (d, 2H), 6.68-6.38 (m, 1H), 6.29 (bs, 1H), 4.80-4.76 (m, 1H), 4.64 (dd, 1H), 2.60 (q, 2H), 2.31-2.29 (m, 1H), 2.12-2.09 (m, 1H), 1.17 (t, 3H). [ES+MS] m/z 322 (M+H)⁺.

### Intermediate 17: (E)-1-(4-ethylphenyl)-4,4-dimethylpent-2-en-1-one

To a solution of 1-(4-ethylphenyl)ethanone (ALDRICH, 3.02 mL, 20.240 mmol) and NaOH (0.081 g, 20.240 mmol) in methanol (25 mL), pivalaldehyde (ALDRICH, 2.199 mL, 20.24 mmol) was added. The reaction was stirred at rt 4 h. LCMS showed reaction was finished. The mixture was diluted with EtOAc, washed with saturated NaHCO₃, water and brine, dried over MgSO₄ and concentrated in vacuo.

Then, the crude material was purified by flash chromatography on silica gel (Merck cartridge, 40 g) and was eluted with mixtures of hexane/EtOAc (90:10). The desired fraction was collected and evaporated to obtain a colorless oil. (2.5 g, 11.56 mmol, 57%). ¹H NMR (400 MHz, CDCl₃) δ ppm: 7.87 (d, 2H), 7.31 (d, 2H), 7.05 (d, 1H), 6.77 (d, 1H), 2.73 (q, 2H), 1.27 (t, 3H), 1.16 (s, 9H).

### Intermediate 18: Ethyl 7-(tert-butyl)-5-(4-ethylphenyl)-4,7-dihydropyrazolo[1,5-a]pyrimidine-3-carboxylate

To a solution of Intermediate 17 (1 g, 4.620 mmol) and ethyl 5-amino-1H-pyrazole-4-carboxylate (ALDRICH, 0.717 g, 4.620 mmol) in 2-(methyloxy)ethanol (10 mL) was added TFA (0.885 mL, 11.560 mmol) with ice-water cooling. The reaction was refluxed overnight. LCMS showed reaction was finished. The mixture was diluted with EtOAc, washed with saturated NaHCO₃, water and brine, dried over MgSO₄ and concentrated in vacuo. Then, the crude material was purified by flash chromatography on silica gel (Merck cartridge, 20 g) and was eluted with mixtures of hexane/EtOAc (80:20). The desired fraction was collected and evaporated to obtain a yellow solid (1.1 g, 3.110 mmol, 67%). ¹H NMR (400 MHz, CDCl₃) δ ppm: 7.73 (s, 1H), 7.44 (d, 2H), 7.25 (d, 2H), 5.09 (dd, 1H), 4.78 (d, 1H), 4.30 (q, 2H), 2.68 (q, 2H), 1.37 (t, 3H), 1.26 (t, 3H), 0.92 (s, 9H). [ES+MS] m/z 353 (M+H)⁺.

### Intermediate 19: Cis-ethyl 7-(tert-butyl)-5-(4-ethylphenyl)-4,5,6,7-tetrahydropyrazolo[1,5-a]pyrimidine-3-carboxylate

The title compound was prepared by a method analogous to that described for Intermediate 2. Intermediate 18 (750 mg, 2.122 mmol) and sodium borohydride (ALDRICH, 281 mg, 7.43 mmol) as starting reactants. The title compound (400 mg, 1.125 mmol, 53%) was obtained as a pale yellow solid. ¹H NMR (400 MHz, CDCl₃) δ ppm: 7.63 (s, 1H), 7.35 (d, 2H), 7.23 (d, 2H), 5.96 (s, 1H), 4.42 (dd, 1H), 4.23 (q, 2H), 4.06 (dd, 1H), 2.68 (q, 2H), 2.26-2.21 (m, 1H), 2.07-2.02 (m, 1H), 1.31 (t, 3H), 1.26 (t, 3H), 1.12 (s, 9H). [ES+MS] m/z 356 (M+H)⁺.

### Intermediate 20: Cis-7-(tert-butyl)-5-(4-ethylphenyl)-4,5,6,7-tetrahydropyrazolo[1,5-a]pyrimidine-3-carboxylic acid

The title compound was prepared by a method analogous to that described for Intermediate 3. Intermediate 19 (368 mg, 1.035 mmol) and potassium hydroxide (0.690 mL, 1.035 mmol) as starting reactants. The title compound (300 mg, 0.910 mmol, 89%). was obtained as a white solid. ¹H NMR (400 MHz, DMSO-d₆) δ ppm: 11.94 (bs, 1H), 7.51 (s, 1H), 7.36 (d, 2H), 7.23 (d, 2H), 5.98 (s, 1H), 4.47 (dd, 1H), 4.06 (dd, 1H), 2.61 (q, 2H), 2.19-2.14 (m, 1H), 1.94-1.85 (m, 1H), 1.17 (t, 3H), 1.06 (s, 9H). [ES+MS] m/z 328 (M+H)⁺.

### Intermediate 21: (5R,7S)-5-(4-ethylphenyl)-7-(trifluoromethyl)-4,5,6,7-tetrahydropyrazolo[1,5-a]pyrimidine-3-carboxylic acid

30 g of cis-5-(4-ethylphenyl)-7-(trifluoromethyl)-4,5,6,7-tetrahydropyrazolo[1,5-a]pyrimidine-3-carboxylic acid (Intermediate 3) was diluted with ethanol (400 mL), then warmed in a sonicating bath at 40° C. for 1 hr. To the hazy solution (contained white flocculent solid) was added heptane (400 mL) and the mixture further sonicated at 40° C. for 1 hr. Ethanol (50 mL) was added and the hazy solution warmed to approx 55° C. with stirring. The warm hazy solution was filtered (porosity 3 filter) before injection. The following conditions were then employed to process the clarified solution on the Varian SD-2 prep HPLC system; Column: Chiralpak IC, 250×50 mm ID, 20 um; Eluent: Heptane+0.1% v/v Acetic acid/Ethanol (90:10 v/v); Flow: 125 ml/min; Temp: 25° C.; Detector: 280 nm, Range 5.

Injection: 37×22 mL & 17 mL of 30.9 mg/mL solution in Heptane/Ethanol (47:53 v/v). (Injection pump set to flow at 46 ml/min for 30 sec which actually delivers 22 mL). Fractions which contained the desired first eluting enantiomer were combined (approx 10 L) and evaporated on the 20 L Buchi using a 10 L flask until the majority of solvent had been removed, then the residue was diluted with ethanol (total vol 200 ml, includes flask rinse) and the pale yellow solution transferred to a 1 L flask. The solvent was removed by evaporation (70 mbar/40° C.) on a lab Buchi and the resulting pale yellow ‘oily’ foam diluted with heptane (100 mL). After re-concentration the residue was triturated with heptane (100 mL) by agitation on a Buchi at 40° C. for 1 hr and occasional scraping with a spatula. The resulting white suspension was allowed to stand at ambient temperature overnight, then filtered. The collected solids were washed with heptane (2×50 mL), sucked dry on the filter for ca. 2 h and then further dried in vacuo at 45° C. for a total of 22.5 h to furnish the first eluting enantiomer, (Intermediate 21) as a white solid. 10 g isolated (36′)/ow/w recovery). Chiral HPLC showed a purity of 99.1% and no evidence of the second eluting enantiomer. [α]_{D}²⁵=+41.4 (c=0.56, EtOH).

Fractions rich in the second eluting enantiomer were combined (approx 20 L) and evaporated on the 20 L Buchi using a 10 L flask until the majority of solvent had been removed, then the residue was diluted with ethanol (total vol 200 ml, includes flask rinse) and the pale yellow solution transferred to a 1 L flask. The solvent was removed by evaporation (70 mbar/40° C.) on lab Buchi and the resulting yellow oil diluted with heptane (100 mL). After re-concentration the residue was triturated with heptane (100 mL) by agitation on a buchi at 40° C. for 1 h and scraping with a spatula. The resulting white suspension was allowed to stand at ambient temperature overnight. The solids were collected by filtration, then washed with heptane (2×50 mL), sucked dry for ca. 1 h and then further dried in vacuo at 45° C. for a total of 65 h to afford the second eluting enantiomer, as a white solid 8.2 g isolated (30% w/w recovery). Chiral HPLC showed a purity of 98.3%. [α]_{D}²⁵=−45.2 (c=0.76, EtOH).

### Intermediate 22: ethyl 7-(1,1-difluoroethyl)-5-(4-ethylphenyl)pyrazolo[1,5-a]pyrimidine-3-carboxylate

The title compound was prepared by a method analogous to that described for Intermediate 1. Ethyl 3-aminopyrazole-4-carboxylate (ALDRICH, 0.646 g, 4.160 mmol) and 1-(4-ethylphenyl)-4,4-difluoropentane-1,3-dione (FLUOROCHEM, 1 g, 4.160 mmol) as starting reactants. The title compound (1.07 g, 2.98 mmol, 72%) was obtained as a pale yellow solid. ¹H NMR (400 MHz, CDCl₃) δ ppm: 8.61 (s, 1H), 8.19 (d, 2H), 7.69 (s, 1H), 7.38 (d, 2H), 4.46 (q, 2H), 2.75 (q, 2H), 2.36 (t, 3H), 1.47 (t, 3H), 1.30 (t, 3H). [ES+MS] m/z 360 (M+H)⁺.

### Intermediate 23: Cis-ethyl 7-(1,1-difluoroethyl)-5-(4-ethylphenyl)-4,5,6,7-tetrahydropyrazolo[1,5-a]pyrimidine-3-carboxylate

The title compound was prepared by a method analogous to that described for Intermediate 2. Intermediate 22 (1.06 g, 2.950 mmol) and sodium borohydride (ALDRICH, 391 mg, 10.32 mmol) as starting reactants. The title compound (996 mg, 2.74 mmol, 89%) was obtained as a white solid. ¹H NMR (400 MHz, CDCl₃) δ ppm: 7.70 (s, 1H), 7.36 (d, 2H), 7.25 (d, 2H), 6.05 (bs, 1H), 4.65-4.51 (m, 2H), 4.28-4.21 (m, 2H), 2.68 (q, 3H), 2.52-2.47 (m, 1H), 2.24-2.15 (m, 1H), 1.79 (t, 3H), 1.32 (t, 3H), 1.26 (t, 3H). [ES+MS] m/z 363 (M+H)⁺.

### Intermediate 24: Cis-7-(1,1-difluoroethyl)-5-(4-ethylphenyl)-4,5,6,7-tetrahydropyrazolo[1,5-a]pyrimidine-3-carboxylic acid

The title compound was prepared by a method analogous to that described for Intermediate 3. Intermediate 23 (980 mg, 2.700 mmol) and potassium hydroxide (3.5 mL, 5.250 mmol) as starting reactants. The title compound (750 mg, 2.556 mmol, 83%) was obtained as a white solid. ¹H NMR (400 MHz, CDCl₃) δ ppm: 7.74 (s, 1H), 7.35 (d, 2H), 7.25 (d, 2H), 6.01 (bs, 1H), 4.64-4.52 (m, 2H), 4.29-4.23 (m, 2H), 2.68 (q, 2H), 2.55-2.48 (m, 1H), 2.26-2.16 (m, 1H), 1.81 (t, 3H), 1.26 (t, 3H). [ES+MS] m/z 336 (M+H)⁺.

### Intermediate 25: ethyl 5-(4-ethylphenyl)-7-hydroxypyrazolo[1,5-a]pyrimidine-3-carboxylate

The title compound was prepared by a method analogous to that described for Intermediate 1. Ethyl 3-aminopyrazole-4-carboxylate (ALDRICH, 1.45 g, 9.350 mmol) and ethyl 3-(4-ethylphenyl)-3-oxopropanoate (AMATEK, 2 g, 9.33 mmol) as starting reactants. The title compound (1.03 g, 3.31 mmol, 35%) was obtained as a pale yellow solid. ¹H NMR (400 MHz, CDCl₃) δ ppm: 8.19 (s, 1H), 7.61 (d, 2H), 7.41 (d, 2H), 6.27 (s, 1H), 4.40 (q, 2H), 2.75 (q, 2H), 1.42 (t, 3H), 1.30 (t, 3H).

### Intermediate 26: ethyl 7-chloro-5-(4-ethylphenyl)pyrazolo[1,5-a]pyrimidine-3-carboxylate

A suspension of Intermediate 25 (1 g, 3.21 mmol) in phosphorus oxychloride (ALDRICH, 5.99 ml, 64.2 mmol) was heated to reflux 2 h. The reaction mixture was slowly added to an elernmeyer containing a mixture of water and ice. The solution was neutralize with Na₂CO₃ and DCM (25 mL) was added. The two layers were separated, the aqueous layer extracted with DCM (2×5 mL). The combined organic layers were washed with water (20 mL) and brine (20 mL), and dried over MgSO₄. The solvent was removed under reduced pressure. The crude product was added to a silica gel column (20 g) and was eluted with (gradient 100% hexane to hexane/AcOEt 80/20). The title compound (390 mg, 1.83 mmol, 37%) was obtained as a pale yellow solid. ¹H NMR (400 MHz, CDCl₃) δ ppm: 8.64 (s, 1H), 8.14 (d, 2H), 7.59 (s, 1H), 7.37 (d, 2H), 4.46 (q, 2H), 2.75 (q, 2H), 1.47 (t, 3H), 1.30 (t, 3H). [ES+MS] m/z 330 (M+H)⁺.

Intermediate 27: ethyl 5-(4-ethylphenyl)-7-isopropylpyrazolo[1,5-a]pyrimidine-3-carboxylate

In a MW vial Intermediate 26 (400 mg, 1.213 mmol), Tetrakis(triphenylphosphine)palladium (ALFAAESAR, 140 mg, 0.121 mmol) was dissolved in Tetrahydrofuran (THF) (5 mL), Then isopropylzinc(II) bromide (ALDRICH, 9.70 mL, 4.85 mmol) was added. The reaction was heated in MW 45 min at 75° C. The reaction was quenched via slowly addition of NH₄Cl aq solution. The reaction mixture was extracted with AcOEt (3×20 mL) the two layers were separated. The combined organic layers were washed with water (20 mL) and brine (20 mL), and dried over MgSO₄. The solvent was removed under reduced pressure. The crude product was added to a silica gel column (10 g) and was eluted with (gradient 100% hexane to Hexane/AcOEt 80/20). The title compound (355 mg, 1.05 mmol, 87%) was obtained as a pale yellow solid. ¹H NMR (400 MHz, CDCl₃) δ ppm: 8.57 (s, 1H), 8.16 (d, 2H), 7.36 (d, 2H), 7.30 (s, 1H), 4.45 (q, 2H), 2.74 (q, 2H), 1.58-1.47 (m, 9H), 1.29 (t, 3H). [ES+MS] m/z 338 (M+H)⁺.

### Intermediate 28: Cis-ethyl 5-(4-ethylphenyl)-7-isopropyl-4,5,6,7-tetrahydropyrazolo[1,5-a]pyrimidine-3-carboxylate

The title compound was prepared by a method analogous to that described for Intermediate 2. Intermediate 27 (350 mg, 1.037 mmol) and sodium borohydride (ALDRICH, 300 mg, 8.32 mmol) as starting reactants. The title compound (245 mg, 0.718 mmol, 69%) was obtained as a pale yellow oil. ¹H NMR (400 MHz, CDCl₃) δ ppm: 7.67 (s, 1H), 7.36 (d, 2H), 7.24 (d, 2H), 5.89 (bs, 1H), 4.51-4.48 (m, 1H), 4.27-4.18 (m, 3H), 2.95-2.90 (m, 1H), 2.68 (q, 3H), 2.14-1.96 (m, 2H), 1.31 (t, 3H), 1.26 (t, 3H), 1.01 (d, 3H), 0.78 (d, 3H). [ES+MS] m/z 342 (M+H)⁺.

### Intermediate 29: Cis-5-(4-ethylphenyl)-7-isopropyl-4,5,6,7-tetrahydropyrazolo[1,5-a]pyrimidine-3-carboxylic acid

The title compound was prepared by a method analogous to that described for Intermediate 3. Intermediate 28 (245 mg, 0.718 mmol) and potassium hydroxide (2.4 mL, 3.59 mmol) as starting reactants. The title compound (224 mg, 0.715 mmol, 100%) was obtained as a white solid. ¹H NMR (400 MHz, DMSO-d₆) δ ppm: 7.51 (s, 1H), 7.35 (d, 2H), 7.24 (d, 2H), 5.97 (bs, 1H), 4.55-4.51 (m, 1H), 4.22-4.17 (m, 1H), 2.79-2.64 (m, 1H), 2.60 (q, 2H), 2.09-2.06 (m, 1H), 1.88-1.80 (m, 1H), 1.17 (t, 3H), 0.94 (d, 3H), 0.70 (d, 3H). [ES+MS] m/z 314 (M+H)⁺.

### EXAMPLES

### Example 1

### Cis-5-(4-ethylphenyl)-N-(4-methoxybenzyl)-7-(trifluoromethyl)-4,5,6,7-tetrahydropyrazolo[1,5-a]pyrimidine-3-carboxamide

### Preparation by Scheme 1

Intermediate 3 (618 mg, 1.821 mmol), HATU (CARBOSYNTH, 831 mg, 2.186 mmol) and N,N-diisopropylethylamine (FLUKA, 0.954 mL, 5.460 mmol) was dissolved in N,N-dimethylformamide (DMF) (15 mL). The reaction mixture was stirred for 30 min at rt then 4-methoxybenzylamine (ALDRICH, 300 mg, 2.186 mmol) was added. The reaction was stirred at 60° C. overnight. The reaction was checked by LCMS and the reaction was completed. The reaction mixture was concentrated under vacuum and partitioned between sodium carbonate (10%) 50 mL and DCM (50 mL) the organic layer was separated, dried over Na2SO4, filtered and concentrated under vacuum. The crude product was added to a silica gel column (30 g) and was eluted with (gradient 100% hexane to hexane/EtOAc 50/50), after collected the appropriated tubes the solvent was removed under vacuum to afford a pale yellow solid (780 mg, 1.703 mmol 93%) ¹H NMR (400 MHz, CDCl₃) δ ppm: 7.50 (s, 1H), 7.35 (d, 2H), 7.27-7.22 (m, 4H), 6.8 (d, 2H), 6.68 (bs, 1H), 4.87-4.81 (m, 1H), 4.53 (dd, 1H), 4.48-4.47 (m, 2H), 3.80 (s, 3H), 2.66 (q, 2H), 2.55-2.52 (m, 1H), 2.42-2.33 (m, 1H), 1.25 (t, 3H). [ES+MS] m/z 459 (M+H)⁺. Example 1 was also purchased from Interbioscreen Ltd.

### Examples 1a and 1b

### Enantiomers a and b of cis-5-(4-ethylphenyl)-N-(4-methoxybenzyl)-7-(trifluoromethyl)-4,5,6,7-tetrahydropyrazolo[1,5-a]pyrimidine-3-carboxamide

### Preparation A

Racemic Example 1 was subjected to semi-preparative high performance liquid chromatography (HPLC) to afford the optically pure enantiomers Example 1a, >95% ee (shorter retention time) and Example 1b, >95% ee (longer retention time). [Column: Chiralpack IC, 250×20 mm, temperature 25° C., mobile phase: hexane/ethanol 90/10, Flow rate: 18 ml/minute, detection wavelength: 254 nm, and injection of 100 mg.

The absolute configuration of the enantiomers a and b was subsequently determined by ab initio vibrational circular dichroism (VCD) with a level of reliability >99%.

Enantiomer 1a (shorter retention time) was determined to be (5R,7S)-5-(4-ethylphenyl)-N-(4-methoxybenzyl)-7-(trifluoromethyl)-4,5,6,7-tetrahydropyrazolo[1,5-a]pyrimidine-3-carboxamide, and Example 1b (longer retention time) was determined to be (5S,7R)-5-(4-ethylphenyl)-N-(4-methoxybenzyl)-7-(trifluoromethyl)-4,5,6,7-tetrahydropyrazolo[1,5-a]pyrimidine-3-carboxamide, In accordance with standard practice the absolute configuration for Intermediate 21 and chiral compounds prepared from this intermediate was assigned on this basis.

### Further preparation of Enantiomers a and b of cis-5-(4-ethylphenyl)-N-(4-methoxybenzyl)-7-(trifluoromethyl)-4,5,6,7-tetrahydropyrazolo[1,5-a]pyrimidine-3-carboxamide

### Preparation B

Racemic Example 1 was subjected to semi-preparative high performance liquid chromatography (HPLC) to afford the optically pure enantiomers 1a: (5R,7S)-5-(4-ethylphenyl)-N-(4-methoxybenzyl)-7-(trifluoromethyl)-4,5,6,7-tetrahydropyrazolo[1,5-a]pyrimidine-3-carboxamide, >95% ee (8 minutes) and 1b: (5S,7R)-5-(4-ethylphenyl)-N-(4-methoxybenzyl)-7-(trifluoromethyl)-4,5,6,7-tetrahydropyrazolo[1,5-a]pyrimidine-3-carboxamide, >95% ee (13 minutes). [Column: Chiralpack IC, 250×20 mm, temperature 25° C., mobile phase: hexane/ethanol 85/15, Flow rate: 18 ml/minute, detection wavelength: 254 nm, and injection of 100 mg.

### Example 2

### Cis-5-(4-ethylphenyl)-N-(4-methylbenzyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (100 mg, 0.295 mmol), HATU (CARBOSYNTH, 134 mg, 0.354 mmol), N,N-diisopropylethylamine (FLUKA, 0.154 mL, 0.884 mmol) and 4-methylbenzylamine (ALDRICH, 46.4 mg, 0.383 mmol) as starting reactants. The title compound (103 mg, 0.233 mmol, 79%) was obtained as a white solid. ¹H NMR (400 MHz, CDCl₃) δ ppm: 7.50 (s, 1H), 7.35 (d, 2H), 7.24-7.21 (m, 4H), 7.15 (d, 2H), 6.68 (bs, 1H), 5.81-5.78 (m, 1H), 4.90-4.80 (m, 1H), 4.55-4.49 (m, 3H), 2.67 (q, 2H), 2.55-2.50 (m, 1H), 2.42-2.36 (m, 1H), 2.34 (s, 3H), 1.25 (t, 3H). [ES+MS] m/z 443 (M+H)⁺.

### Example 3

### Cis-5-(4-ethylphenyl)-N-(3-methylbenzyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (100 mg, 0.295 mmol), HATU (CARBOSYNTH, 134 mg, 0.354 mmol), N,N-diisopropylethylamine (FLUKA, 0.154 mL, 0.884 mmol), 3-methylbenzylamine (ALDRICH, 46.4 mg, 0.383 mmol) as starting reactants. The title compound (105 mg, 0.237 mmol, 81%) was obtained as a white solid. ¹H NMR (400 MHz, CDCl₃) δ ppm: 7.52 (s, 1H), 7.35 (d, 2H), 7.24-7.21 (m, 3H), 7.13-7.09 (m, 3H), 6.68 (bs, 1H), 5.83-5.80 (m, 1H), 4.87-4.81 (m, 1H), 4.56-4.50 (m, 3H), 2.67 (q, 2H), 2.52-2.33 (m, 4H), 1.25 (t, 3H). [ES+MS] m/z 443 (M+H)⁺.

### Example 4

### Cis-5-(4-ethylphenyl)-N-((5-methylfuran-2-yl)methyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (100 mg, 0.295 mmol), HATU (CARBOSYNTH, 134 mg, 0.354 mmol), N,N-diisopropylethylamine (FLUKA, 0.154 mL, 0.884 mmol) and 5-methylfurfurylamine (ALDRICH, 39.3 mg, 0.354 mmol) as starting reactants. The title compound (103 mg, 0.238 mmol, 81%) was obtained as a white solid. ¹H NMR (400 MHz, CDCl₃) δ ppm: 7.53 (s, 1H), 7.34 (d, 2H), 7.23 (d, 2H), 6.65 (bs, 1H), 6.11 (d, 1H), 5.92-5.89 (m, 1H), 5.80-5.77 (m, 1H), 4.52 (dd, 1H), 4.48-4.46 (m, 1H), 2.67 (q, 2H), 2.54-2.50 (m, 1H), 2.41-2.32 (m, 1H), 2.28 (s, 3H), 1.25 (t, 3H). [ES+MS] m/z 433 (M+H)⁺.

### Example 5

### Cis-N-(cyclohexylmethyl)-5-(4-ethylphenyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (100 mg, 0.295 mmol), HATU (CARBOSYNTH, 134 mg, 0.354 mmol), N,N-diisopropylethylamine (FLUKA, 0.154 mL, 0.884 mmol), 5-cyclohexanemethylamine (ALDRICH, 40 mg, 0.354 mmol) as starting reactants. The title compound (80 mg, 0.184 mmol, 62%) was obtained as a white solid. ¹H NMR (400 MHz, CDCl₃) δ ppm: 7.52 (s, 1H), 7.34 (d, 2H), 7.22 (d, 2H), 6.66 (bs, 1H), 5.61-5.58 (m, 1H), 4.87-4.81 (m, 1H), 4.54-4.50 (m, 1H), 3.20-3.17 (m, 2H), 2.66 (q, 2H), 2.54-2.49 (m, 1H), 1.67-1.65 (m, 5H), 1.55-1.49 (m, 1H), 1.26-1.14 (m, 6H), 0.99-0.90 (m, 2H). [ES+MS] m/z 435 (M+H)⁺.

### Example 6

### Cis-5-(4-ethylphenyl)-7-(trifluoromethyl)-N-(4-(trifluoromethyl)benzyl)-4,5,6,7-tetrahydropyrazolo[1,5-a]pyrimidine-3-carboxamide

The title compound was prepared by a method analogous to that described for Example 1. Intermediate 3 (100 mg, 0.295 mmol), HATU (CARBOSYNTH, 134 mg, 0.354 mmol), N,N-diisopropylethylamine (FLUKA, 0.154 mL, 0.884 mmol) and 4-(Trifluoromethyl)benzylamine (ALDRICH, 67.1 mg, 0.383 mmol) as starting reactants. The title compound (80 mg, 0.161 mmol, 55%) was obtained as a white solid. ¹H NMR (400 MHz, CDCl₃) δ ppm: 7.59 (d, 2H), 7.56 (s, 1H), 7.44 (d, 2H), 7.35 (d, 2H), 7.23 (d, 2H), 6.64 (bs, 1H), 5.98-5.95 (m, 1H), 4.88-4.82 (m, 1H), 4.60-4.52 (m, 3H), 2.67 (q, 2H), 2.57-2.50 (m, 1H), 2.42-2.33 (m, 1H), 1.24 (t, 3H). [ES+MS] m/z 497 (M+H)⁺.

### Example 7

### Cis-(4-ethylphenyl)-N-(2-furanylmethyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (100 mg, 0.295 mmol), HATU (CARBOSYNTH, 134 mg, 0.354 mmol), N,N-diisopropylethylamine (FLUKA, 0.154 mL, 0.884 mmol) and furfurylamine (ALDRICH, 37.2 mg, 0.383 mmol) as starting reactants. The title compound (95 mg, 0.227 mmol, 77%) was obtained as a white solid. ¹H NMR (400 MHz, CDCl₃) δ ppm: 7.53 (s, 1H), 7.37-7.33 (m, 3H), 7.23 (d, 2H), 6.65 (bs, 1H), 6.33 (dd, 1H), 6.27-6.25 (m, 1H), 5.85-5.82 (m, 1H), 4.86-4.81 (m, 1H), 4.59-4.48 (m, 3H), 3.80 (s, 3H), 2.67 (q, 2H), 2.55-2.49 (m, 1H), 2.41-2.32 (m, 1H), 1.25 (t, 3H). [ES+MS] m/z 419 (M+H)⁺.

### Examples 7a and 7b

### Enantiomers a and b of cis-5-(4-ethylphenyl)-N-(2-furanylmethyl)-7-(trifluoromethyl)-4,5,6,7-tetrahydropyrazolo[1,5-a]pyrimidine-3-carboxamide

### Preparation A

Racemic Example 7 was separated into its enantiomers using an analogous procedure to that described for separation of Example 1, Preparation A to obtain

Example 7a (5R,7S)-5-(4-ethylphenyl)-N-(2-furanylmethyl)-7-(trifluoromethyl)-4,5,6,7-tetrahydropyrazolo[1,5-a]pyrimidine-3-carboxamide
>95% ee (shorter retention time) and
Example 7b (5S,7R)-5-(4-ethylphenyl)-N-(2-furanylmethyl)-7-(trifluoromethyl)-4,5,6,7-tetrahydropyrazolo[1,5-a]pyrimidine-3-carboxamide
>95% ee (longer retention time).

### Further preparation of: Enantiomers a and b of cis-5-(4-ethylphenyl)-N-(2-furanylmethyl)-7-(trifluoromethyl)-4,5,6,7-tetrahydropyrazolo[1,5-a]pyrimidine-3-carboxamide

Preparation B Racemic Example 7 was subjected to semi-preparative high performance liquid chromatography (HPLC) to afford the optically pure enantiomers 7a: (5R,7S)-5-(4-ethylphenyl)-N-(2-furanylmethyl)-7-(trifluoromethyl)-4,5,6,7-tetrahydropyrazolo[1,5-a]pyrimidine-3-carboxamide, >95% ee (13 minutes) and 7b: (5S,7R)-5-(4-ethylphenyl)-N-(2-furanylmethyl)-7-(trifluoromethyl)-4,5,6,7-tetrahydropyrazolo[1,5-a]pyrimidine-3-carboxamide, >95% ee (19 minutes). [Column: Chiralpack IC, 250×20 mm, temperature 25° C., mobile phase: hexane/ethanol 93/7, Flow rate: 18 ml/minute, detection wavelength: 254 nm, and injection of 100 mg.

### Example 8

### Cis-5-(4-ethylphenyl)-N-{[3-(methyloxy)phenyl]methyl}-7-(trifluoromethyl)-4,5,6,7-tetrahydropyrazolo[1,5-a]pyrimidine-3-carboxamide

The title compound was prepared by a method analogous to that described for Example 1. Intermediate 3 (60 mg, 0.177 mmol), HATU (CARBOSYNTH, 81 mg, 0.212 mmol), N,N-diisopropylethylamine (FLUKA, 0.093 mL, 0.532 mmol) and 3-methoxybenzylamine (ALFAAESAR, 29.1 mg, 0.212 mmol) as starting reactants. The title compound (58 mg, 0.127 mmol, 71%) was obtained as a white solid. ¹H NMR (400 MHz, CDCl₃) δ ppm: 7.53 (s, 1H), 7.35 (d, 2H), 7.28-7.22 (m, 3H), 6.93-6.89 (m, 1H), 6.87-6.85 (m, 1H), 6.84-6.80 (m, 1H), 6.68 (bs, 1H), 5.85-5.80 (m, 1H), 4.89-4.80 (m, 1H), 4.56-4.51 (m, 3H), 3.80 (s, 3H), 2.67 (q, 2H), 2.56-2.50 (m, 1H), 2.42-2.33 (m, 1H), 1.25 (t, 3H). [ES+MS] m/z 459 (M+H)⁺.

### Example 9

### Cis-N-(3-chloro-4-methylbenzyl)-5-(4-ethylphenyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (100 mg, 0.295 mmol), HATU (CARBOSYNTH, 134 mg, 0.354 mmol), N,N-diisopropylethylamine (FLUKA, 0.257 mL, 1.474 mmol) and 3-chloro-4-methylbencylamine (ACROS, 55.0 mg, 0.354 mmol) as starting reactants. The title compound (50.2 mg, 0.105 mmol, 35%) was obtained as a white solid. ¹H NMR (400 MHz, CDCl₃) δ ppm: 7.53 (s, 1H), 7.35 (d, 2H), 7.31-7.29 (m, 1H), 7.23 (d, 2H), 7.20-7.18 (m, 1H), 7.12-7.10 (m, 1H), 6.66 (bs, 1H), 5.85-5.82 (m, 1H), 4.89-4.80 (m, 1H), 4.56-4.52 (m, 1H), 4.49-4.44 (m, 2H), 2.67 (q, 2H), 2.56-2.50 (m, 1H), 2.42-2.33 (m, 4H), 1.25 (t, 3H). [ES+MS] m/z 477 (M+H)⁺.

### Example 10

### Cis-5-(4-ethylphenyl)-N-[(1-methyl-1H-pyrrol-2-yl)methyl]-7-(trifluoromethyl)-4,5,6,7-tetrahydropyrazolo[1,5-a]pyrimidine-3-carboxamide

The title compound was prepared by a method analogous to that described for Example 1. Intermediate 3 (60 mg, 0.177 mmol), HATU (CARBOSYNTH, 81 mg, 0.212 mmol), N,N-diisopropylethylamine (FLUKA, 0.093 mL, 0.532 mmol) and (1-methyl-1H-pyrrol-2-yl)methylamine (MAYBRIDGE, 23.37 mg, 0.212 mmol) as starting reactants. The title compound (54.8 mg, 0.127 mmol, 71%) was obtained as a white solid. ¹H NMR (400 MHz, CDCl₃) δ ppm: 7.49 (s, 1H), 7.35 (d, 2H), 7.24 (d, 2H), 6.66 (bs, 1H), 6.65-6.63 (m, 1H), 6.14-6.13 (m, 1H), 6.10-6.08 (m, 1H), 5.57-5.55 (m, 1H), 4.89-4.80 (m, 1H), 4.59-4.47 (m, 3H), 3.59 (s, 3H), 2.67 (q, 2H), 2.56-2.50 (m, 1H), 2.42-2.33 (m, 1H), 1.25 (t, 3H). [ES+MS] m/z 432 (M+H)⁺.

### Example 11

### Cis-N-(4-(dimethylamino)benzyl)-5-(4-ethylphenyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (100 mg, 0.295 mmol), HATU (CARBOSYNTH, 134 mg, 0.354 mmol), N,N-diisopropylethylamine (FLUKA, 0.257 ml, 1.474 mmol) and 4-(dimethylamino)benzylamine dihydrochloride (ALDRICH, 79 mg, 0.354 mmol) as starting reactants. The title compound (43 mg, 0.091 mmol, 31%) was obtained as a yellow solid. ¹H NMR (400 MHz, CDCl₃) δ ppm: 7.48 (s, 1H), 7.36 (d, 2H), 7.24-7.20 (m, 4H), 6.73-6.68 (m, 3H), 5.71-5.68 (m, 1H), 4.89-4.80 (m, 1H), 4.55-4.51 (m, 1H), 4.44-4.42 (m, 2H), 2.94 (s, 6H), 2.70-2.64 (m, 2H), 2.49-2.25 (m, 1H), 2.42-2.33 (m, 1H), 1.25 (t, 3H). [ES+MS] m/z 472 (M+H)⁺.

### Example 12

### Cis-5-(4-ethylphenyl)-N-(furan-3-ylmethyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (100 mg, 0.295 mmol), HATU (CARBOSYNTH, 134 mg, 0.354 mmol), N,N-diisopropylethylamine (FLUKA, 0.257 mL, 1.474 mmol) and furan-3-ylmethanamine hydrochloride (MAYBRIDGE, 47.2 mg, 0.354 mmol) as starting reactants. The title compound (95 mg, 0.227 mmol, 77%) was obtained as a white solid. ¹H NMR (400 MHz, CDCl₃) δ ppm: 7.51 (s, 1H), 7.41-7.39 (m, 2H), 7.35 (d, 2H), 7.23 (d, 2H), 6.66 (bs, 1H), 6.40-6.39 (m, 1H), 5.70-5.68 (m, 1H), 4.88-4.80 (m, 1H), 4.56-4.52 (m, 1H), 4.38 (d, 2H), 2.67 (q, 2H), 2.56-2.50 (m, 1H), 2.42-2.33 (m, 1H), 1.25 (t, 3H). [ES+MS] m/z 419 (M+H)⁺.

### Example 13

### Cis-5-(4-ethylphenyl)-N-(1-naphthalenylmethyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (60 mg, 0.177 mmol), HATU (CARBOSYNTH, 81 mg, 0.212 mmol), N,N-diisopropylethylamine (FLUKA, 0.93 mL, 0.532 mmol) and 1-naphtalenemethylamine (ALDRICH, 33.4 mg, 0.212 mmol) as starting reactants. The title compound (53.8 mg, 0.112 mmol, 64%) was obtained as a white solid. ¹H NMR (400 MHz, CDCl₃) δ ppm: 8.06 (d, 1H), 7.91-7.83 (m, 2H), 7.57-7.43 (m, 5H), 7.38 (d, 2H), 7.25 (d, 2H), 6.73 (bs, 1H), 5.78-5.75 (m, 1H), 5.06-4.94 (m, 2H), 4.86-4.81 (m, 1H), 4.58-4.54 (m, 1H), 2.65 (q, 2H), 2.56-2.51 (m, 1H), 2.43-2.34 (m, 1H), 1.25 (t, 3H). [ES+MS] m/z 479 (M+H)⁺.

### Example 14

### Cis-5-(4-ethylphenyl)-7-(trifluoromethyl)-N-{[3-(trifluoromethyl)phenyl]methyl}-4,5,6,7-tetrahydropyrazolo[1,5-a]pyrimidine-3-carboxamide

The title compound was prepared by a method analogous to that described for Example 1. Intermediate 3 (100 mg, 0.295 mmol), HATU (CARBOSYNTH, 134 mg, 0.354 mmol), N,N-diisopropylethylamine (FLUKA, 0.154 mL, 0.884 mmol) and 3-(trifluoromethyl)benzylamine (ALDRICH, 67.1 mg, 0.383 mmol) as starting reactants. The title compound (91 mg, 0.183 mmol, 62%) was obtained as a white solid. ¹H NMR (400 MHz, CDCl₃) δ ppm: 7.57-7.52 (m, 4H), 7.48-7.44 (m, 1H), 7.35 (d, 2H), 7.23 (d, 2H), 6.65 (bs, 1H), 5.95-5.92 (m, 1H), 4.89-4.81 (m, 1H), 4.65-4.53 (m, 3H), 2.67 (q, 2H), 2.56-2.50 (m, 1H), 2.43-2.34 (m, 1H), 1.25 (t, 3H). [ES+MS] m/z 497 (M+H)⁺.

### Example 15

### Cis-N-benzyl-5-(4-ethylphenyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (80 mg, 0.236 mmol), HATU (CARBOSYNTH, 108 mg, 0.283 mmol), N,N-diisopropylethylamine (FLUKA, 0.124 mL, 0.707 mmol) and phenylmethanamine (ALDRICH, 32.8 mg, 0.307 mmol) as starting reactants. The title compound (93 mg, 0.217 mmol, 92%) was obtained as a white solid. ¹H NMR (400 MHz, CDCl₃) δ ppm: 7.52 (s, 1H), 7.36-7.28 (m, 7H), 7.23 (d, 2H), 6.68 (bs, 1H), 5.86-5.83 (m, 1H), 4.87-4.81 (m, 1H), 4.56-4.52 (m, 3H), 2.67 (q, 2H), 2.56-2.52 (m, 1H), 2.43-2.33 (m, 1H), 1.25 (t, 3H). [ES+MS] m/z 429 (M+H)⁺.

### Example 16

### Cis-N-[(3,4-difluorophenyl)methyl]-5-(4-ethylphenyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (100 mg, 0.295 mmol), HATU (CARBOSYNTH, 134 mg, 0.354 mmol), N,N-diisopropylethylamine (FLUKA, 0.154 mL, 0.884 mmol) and 3,4-difluorobenzylamine (ALDRICH, 71.69 mg, 0.501 mmol) as starting reactants. The title compound (113.3 mg, 0.244 mmol, 83%) was obtained as a white solid. ¹H NMR (400 MHz, CDCl₃) δ ppm: 7.55 (s, 1H), 7.35 (d, 2H), 7.23 (d, 2H), 7.17-7.08 (m, 2H), 7.06-7.01 (m, 1H), 6.63 (bs, 1H), 5.95-5.92 (m, 1H), 4.89-4.80 (m, 1H), 4.56-4.48 (m, 3H), 2.65 (q, 2H), 2.56-2.51 (m, 1H), 2.42-2.33 (m, 1H), 1.25 (t, 3H). [ES+MS] m/z 465 (M+H)⁺.

### Example 17

### Cis-N-[(3,5-difluorophenyl)methyl]-5-(4-ethylphenyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (100 mg, 0.295 mmol), HATU (CARBOSYNTH, 134 mg, 0.354 mmol), N,N-diisopropylethylamine (FLUKA, 0.154 mL, 0.884 mmol) and 3,5-difluorobenzylamine (ALDRICH, 84.3 mg, 0.59 mmol) as starting reactants. The title compound (54.3 mg, 0.117 mmol, 40%) was obtained as a white solid. ¹H NMR (400 MHz, CDCl₃) δ ppm: 7.57 (s, 1H), 7.35 (d, 2H), 7.23 (d, 2H), 6.87-6.83 (m, 2H), 6.73-6.70 (m, 1H), 6.63 (bs, 1H), 5.95-5.92 (m, 1H), 4.89-4.80 (m, 1H), 4.58-4.47 (m, 3H), 2.65 (q, 2H), 2.56-2.51 (m, 1H), 2.43-2.34 (m, 1H), 1.25 (t, 3H). [ES+MS] m/z 465 (M+H)⁺.

### Example 18

### Cis-N-(2,3-dihydro-1,4-benzodioxin-6-ylmethyl)-5-(4-ethylphenyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (100 mg, 0.295 mmol), HATU (CARBOSYNTH, 134 mg, 0.354 mmol), N,N-diisopropylethylamine (FLUKA, 0.154 mL, 0.884 mmol) and 2,3-dihydro-1,4-benzodioxin-6-ylmethylamine (MAYBRIDGE, 58.4 mg, 0.354 mmol). The title compound (113.2 mg, 0.233 mmol, 79%) was obtained as a white solid. ¹H NMR (400 MHz, CDCl₃) δ ppm: 7.51 (s, 1H), 7.35 (d, 2H), 7.23 (d, 2H), 6.84-6.78 (m, 3H), 6.67 (bs, 1H), 5.78-5.75 (m, 1H), 4.87-4.81 (m, 1H), 4.55-4.52 (m, 1H), 4.47-4.38 (m, 2H), 4.25 (s, 4H), 2.65 (q, 2H), 2.56-2.51 (m, 1H), 2.43-2.34 (m, 1H), 1.25 (t, 3H). [ES+MS] m/z 487 (M+H)⁺.

### Example 19

### Cis-5-(4-ethylphenyl)-N-((6-methylpyridin-3-yl)methyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (100 mg, 0.295 mmol), HATU (CARBOSYNTH, 134 mg, 0.354 mmol), N,N-diisopropylethylamine (FLUKA, 0.154 mL, 0.884 mmol) and (6-methylpyridin-3-yl)methanamine (ABCHEM, 46.8 mg, 0.383 mmol) as starting reactants. The title compound (100 mg, 0.225 mmol, 77%) was obtained as a pale yellow solid. ¹H NMR (400 MHz, CDCl₃) δ ppm: 8.44 (s, 1H), 7.58-7.54 (m, 2H), 7.35 (d, 2H), 7.23 (d, 2H), 7.12 (d, 2H), 6.64 (bs, 1H), 5.97-5.94 (m, 1H), 4.87-4.81 (m, 1H), 4.56-4.50 (m, 3H), 2.67 (q, 2H), 2.54-2.50 (m, 4H), 1.25 (t, 3H); [ES+MS] m/z 444 (M+H)⁺.

### Example 20

### Cis-5-(4-ethylphenyl)-N-((5-methylpyridin-2-yl)methyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (100 mg, 0.295 mmol), HATU (CARBOSYNTH, 134 mg, 0.354 mmol), N,N-diisopropylethylamine (FLUKA, 0.154 mL, 0.884 mmol) and (5-methylpyridin-2-yl)methanamine hydrochloride (ALLWEYS, 60.8 mg, 0.383 mmol) as starting reactants. The title compound (97 mg, 0.219 mmol, 74%) was obtained as a pale yellow solid. ¹H NMR (400 MHz, CDCl₃) δ ppm: 8.42 (s, 1H), 7.67 (s, 1H), 7.51 (d, 1H), 7.39 (d, 2H), 7.30-7.22 (m, 4H), 6.78 (m, 1H), 6.69 (bs, 1H), 4.87-4.81 (m, 1H), 4.63 (d, 2H), 4.56 (d, 1H), 2.71 (q, 2H), 2.61-2.54 (m, 1H), 2.45-2.39 (m, 1H), 2.37 (s, 3H), 1.29 (t, 3H); [ES+MS] m/z 444 (M+H)⁺.

### Example 21

### Cis-5-(4-bromophenyl)-N-(4-methylbenzyl)-7-(trifluoromethyl)-4,5,6,7-tetrahydropyrazolo[1,5-a]pyrimidine-3-carboxamide

The title compound was prepared by a method analogous to that described for Example 1. Intermediate 6 (100 mg, 0.256 mmol), HATU (CARBOSYNTH, 117 mg, 0.308 mmol), N,N-diisopropylethylamine (FLUKA, 0.134 mL, 0.769 mmol) and 4-methylbenzylamine (ALDRICH, 31.1 mg, 0.256 mmol). The title compound (63.9 mg, 0.130 mmol, 50%) was obtained as a pale yellow solid. ¹H NMR (400 MHz, CDCl₃) δ ppm: 7.55-7.52 (m, 3H), 7.33 (d, 2H), 7.22 (d, 2H); 7.16 (d, 2H), 6.71 (s, 1H), 5.84-5.81 (m, 1H), 4.88-4.80 (m, 1H), 4.55-4.49 (m, 3H), 2.54-2.49 (m, 1H), 2.38-2.29 (m, 4H). [ES+MS] m/z 493 (M+H)⁺.

### Example 22

### Cis-N-(benzo[d][1,3]dioxol-5-ylmethyl)-5-(4-bromophenyl)-7-(trifluoromethyl)-4,5,6,7-tetrahydropyrazolo[1,5-a]pyrimidine-3-carboxamide

The title compound was prepared by a method analogous to that described for Example 1. Intermediate 6 (340 mg, 0.871 mmol), HATU (CARBOSYNTH, 398 mg, 1.046 mmol), N,N-diisopropylethylamine (FLUKA, 0.457 mL, 2.610 mmol) and piperonylamine (ALDRICH, 171 mg, 1.133 mmol). The title compound (305 mg, 0.583 mmol, 67%) was obtained as a pale yellow solid. ¹H NMR (400 MHz, CDCl₃) δ ppm: 7.55-7.53 (m, 3H), 7.33 (d, 2H), 6.82 (s, 1H), 6.77 (s, 2H), 6.70 (bs, 1H), 5.95 (s, 2H), 5.85-5.82 (m, 1H), 4.88-4.80 (m, 1H), 4.56-4.52 (m, 1H), 4.49-4.40 (m, 2H), 2.55-2.50 (m, 1H), 2.38-2.29 (m, 1H). [ES+MS] m/z 523 (M+H)⁺.

### Example 23

### Cis-5-(4-ethylphenyl)-7-methyl-N-{[4-(methyloxy)phenyl]methyl}-4,5,6,7-tetrahydropyrazolo[1,5-a]pyrimidine-3-carboxamide

The title compound was prepared by a method analogous to that described for Example 1. Intermediate 9 (89 mg, 0.312 mmol), HATU (CARBOSYNTH, 142 mg, 0.374 mmol), N,N-diisopropylethylamine (FLUKA, 0.163 mL, 0.936 mmol) and 4-methoxybenzylamine (ALDRICH, 55.6 mg, 0.405 mmol) as starting reactants. The title compound (93 mg, 0.230 mmol, 73%) was obtained as a white solid. ¹H NMR (400 MHz, CDCl₃) δ ppm: 7.41 (s, 1H), 7.34 (d, 2H), 7.25 (d, 2H), 7.20 (d, 2H), 6.87 (d, 2H), 6.46 (bs, 1H), 5.74-5.71 (m, 1H), 4.55-4.42 (m, 3H), 4.33-4.28 (m, 1H), 3.80 (s, 3H) 2.66 (q, 2H), 2.33-2.29 (m, 1H), 2.09-1.97 (m, 1H), 1.59 (d, 3H), 1.24 (t, 3H). [ES+MS] m/z 405 (M+H)⁺.

### Example 24

### Cis-N-(3-methylbenzyl)-5-(p-tolyl)-7-(trifluoromethyl)-4,5,6,7-tetrahydropyrazolo[1,5-a]pyrimidine-3-carboxamide

The title compound was prepared by a method analogous to that described for Example 1. Intermediate 13 (100 mg, 0.307 mmol), HATU (CARBOSYNTH, 140 mg, 0.369 mmol), N,N-diisopropylethylamine (FLUKA, 0.161 mL, 0.922 mmol) and 3-methylbenzylamine (ALDRICH, 48.4 mg, 0.400 mmol) as starting reactants. The title compound (105 mg, 0.245 mmol, 80%) was obtained as a white solid. ¹H NMR (400 MHz, CDCl₃) δ ppm: 7.52 (s, 1H), 7.33 (d, 2H), 7.24-7.19 (m, 3H), 7.13-7.09 (m, 3H), 6.68 (bs, 1H), 5.84-5.81 (m, 1H), 4.87-4.81 (m, 1H), 4.55-4.50 (m, 3H), 2.59-2.54 (m, 1H), 2.41-2.32 (m, 7H). [ES+MS] m/z 429 (M+H)⁺.

### Example 25

### Cis-N-(4-methylbenzyl)-5-(p-tolyl)-7-(trifluoromethyl)-4,5,6,7-tetrahydropyrazolo[1,5-a]pyrimidine-3-carboxamide

The title compound was prepared by a method analogous to that described for Example 1. Intermediate 13 (100 mg, 0.307 mmol), HATU (CARBOSYNTH, 140 mg, 0.369 mmol), N,N-diisopropylethylamine (FLUKA, 0.161 mL, 0.922 mmol) and 4-methylbenzylamine (ALDRICH, 48.4 mg, 0.400 mmol) as starting reactants. The title compound (103 mg, 0.240 mmol, 78%) was obtained as a white solid. ¹H NMR (400 MHz, CDCl₃) δ ppm: 7.51 (s, 1H), 7.32 (d, 2H), 7.23-7.19 (m, 4H), 7.15 (d, 2H), 6.68 (bs, 1H), 5.81-5.79 (m, 1H), 4.87-4.81 (m, 1H), 4.54-4.49 (m, 3H), 2.54-2.49 (m, 1H), 2.41-2.32 (m, 7H). [ES+MS] m/z 429 (M+H)⁺.

### Example 26

### Cis-N-(cyclohexylmethyl)-5-(p-tolyl)-7-(trifluoromethyl)-4,5,6,7-tetrahydropyrazolo[1,5-a]pyrimidine-3-carboxamide

The title compound was prepared by a method analogous to that described for Example 1. Intermediate 13 (100 mg, 0.307 mmol), HATU (CARBOSYNTH, 140 mg, 0.369 mmol), N,N-diisopropylethylamine (FLUKA, 0.161 mL, 0.922 mmol), 5-cyclohexanemethylamine (ALDRICH, 41.8 mg, 0.369 mmol) as starting reactants. The title compound (98 mg, 0.233 mmol, 76%) was obtained as a white solid. ¹H NMR (400 MHz, CDCl₃) δ ppm: 7.52 (s, 1H), 7.32 (d, 2H), 7.19 (d, 2H), 6.66 (bs, 1H), 5.61-5.58 (m, 1H), 4.86-4.81 (m, 1H), 3.20-3.17 (m, 2H), 2.53-2.49 (m, 1H), 2.40-2.31 (m, 3H), 1.77-1.68 (m, 5H), 1.28-1.14 (m, 3H), 0.99-0.90 (m, 2H). [ES+MS] m/z 421 (M+H)⁺.

### Example 27

### Cis-N-(3-methoxybenzyl)-5-(p-tolyl)-7-(trifluoromethyl)-4,5,6,7-tetrahydropyrazolo[1,5-a]pyrimidine-3-carboxamide

The title compound was prepared by a method analogous to that described for Example 1. Intermediate 13 (100 mg, 0.307 mmol), HATU (CARBOSYNTH, 140 mg, 0.369 mmol), N,N-diisopropylethylamine (FLUKA, 0.161 mL, 0.922 mmol) and 3-methoxybenzylamine (ALFAAESAR, 50.6 mg, 0.369 mmol) as starting reactants. The title compound (91 mg, 0.205 mmol, 67%) was obtained as a white solid. ¹H NMR (400 MHz, CDCl₃) δ ppm: 7.53 (s, 1H), 7.32 (d, 2H), 7.20 (d, 2H), 6.92-6.90 (m, 1H), 6.87-6.85 (m, 1H), 6.84-6.80 (m, 1H), 6.67 (bs, 1H), 5.85-5.82 (m, 1H), 4.87-4.81 (m, 1H), 4.56-4.51 (m, 3H), 3.80 (s, 3H), 2.56-2.50 (m, 1H), 2.42-2.33 (m, 4H). [ES+MS] m/z 445 (M+H)⁺.

### Example 28

### Cis-5-(p-tolyl)-7-(trifluoromethyl)-N-(4-(trifluoromethyl)benzyl)-4,5,6,7-tetrahydropyrazolo[1,5-a]pyrimidine-3-carboxamide

The title compound was prepared by a method analogous to that described for Example 1. Intermediate 13 (100 mg, 0.307 mmol), HATU (CARBOSYNTH, 140 mg, 0.369 mmol), N,N-diisopropylethylamine (FLUKA, 0.161 mL, 0.922 mmol) and 3-chloro-4-methylbenzylamine (ALDRICH, 96.9 mg, 0.554 mmol) as starting reactants. The title compound (76.8 mg, 0.159 mmol, 52%) was obtained as a white solid. ¹H NMR (400 MHz, CDCl₃) δ ppm: 7.59 (d, 2H), 7.56 (s, 1H), 7.44 (d, 2H), 7.32 (d, 2H), 7.20 (d, 2H), 6.64 (bs, 1H), 5.98-5.95 (m, 1H), 4.87-4.82 (m, 1H), 4.61-4.51 (m, 3H), 2.55-2.50 (m, 1H), 2.41-2.32 (m, 4H). [ES+MS] m/z 483 (M+H)⁺.

### Example 29

### Cis-N-(3-chloro-4-methylbenzyl)-5-(p-tolyl)-7-(trifluoromethyl)-4,5,6,7-tetrahydropyrazolo[1,5-a]pyrimidine-3-carboxamide

The title compound was prepared by a method analogous to that described for Example 1. Intermediate 13 (100 mg, 0.307 mmol), HATU (CARBOSYNTH, 140 mg, 0.369 mmol), N,N-diisopropylethylamine (FLUKA, 0.161 mL, 0.922 mmol) and 3-chloro-4-methylbenzylamine (ACROS, 110 mg, 0.707 mmol) as starting reactants. The title compound (69.2 mg, 0.149 mmol, 49%) was obtained as a white solid. ¹H NMR (400 MHz, CDCl₃) δ ppm: 7.53 (s, 1H), 7.33-7.30 (m, 3H), 7.21-7.18 (m, 3H), 7.12-7.10 (m, 1H), 6.65 (bs, 1H), 5.85-5.82 (m, 1H), 4.88-4.80 (m, 1H), 4.55-4.43 (m, 3H), 2.55-2.49 (m, 1H), 2.41-2.32 (m, 7H). [ES+MS] m/z 463 (M+H)⁺.

### Example 30

### Cis-N-(3,5-difluorobenzyl)-5-(p-tolyl)-7-(trifluoromethyl)-4,5,6,7-tetrahydropyrazolo[1,5-a]pyrimidine-3-carboxamide

The title compound was prepared by a method analogous to that described for Example 1. Intermediate 13 (100 mg, 0.307 mmol), HATU (CARBOSYNTH, 140 mg, 0.369 mmol), N,N-diisopropylethylamine (FLUKA, 0.161 mL, 0.922 mmol) and 3,5-difluorobenzylamine (ALDRICH, 79 mg, 0.553 mmol) as starting reactants. The title compound (65 mg, 0.144 mmol, 47%) was obtained as a pale yellow solid. ¹H NMR (400 MHz, CDCl₃) δ ppm: 8.51 (t, 1H), 7.87 (s, 1H), 7.32 (d, 2H), 7.20 (d, 2H), 7.10-7.04 (m, 1H), 6.99-6.94 (m, 2H), 6.54 (bs, 1H), 5.29-5.23 (m, 1H), 4.63-4.59 (m, 1H), 4.38-4.36 (m, 2H), 2.30 (bs, 3H), 2.18-2.09 (m, 3H). [ES+MS] m/z 451 (M+H)⁺.

### Example 31

### Cis-N-(3,4-difluorobenzyl)-5-(p-tolyl)-7-(trifluoromethyl)-4,5,6,7-tetrahydropyrazolo[1,5-a]pyrimidine-3-carboxamide

The title compound was prepared by a method analogous to that described for Example 1. Intermediate 13 (100 mg, 0.307 mmol), HATU (CARBOSYNTH, 227 mg, 0.369 mmol), N,N-diisopropylethylamine (FLUKA, 0.157 mL, 0.922 mmol) and 3,4-difluorobenzylamine (ALDRICH, 66 mg, 0.461 mmol) as starting reactants. The title compound (97 mg, 0.215 mmol, 70%) was obtained as a pale yellow solid. ¹H NMR (400 MHz, CDCl₃) δ ppm: 7.55 (s, 1H), 7.32 (d, 2H), 7.21 (d, 2H), 7.15-7.10 (m, 2H), 7.08-7.02 (m, 2H), 6.33 (bs, 1H), 5.91 (t, 1H), 4.89-4.80 (m, 1H), 4.55-4.44 (m, 3H), 2.55-2.49 (m, 1H), 2.41-2.32 (m, 4H). [ES+MS] m/z 451 (M+H)⁺.

### Example 32

### Cis-7-(difluoromethyl)-5-(4-ethylphenyl)-N-(4-methoxybenzyl)-4,5,6,7-tetrahydropyrazolo[1,5-a]pyrimidine-3-carboxamide

The title compound was prepared by a method analogous to that described for Example 1. Intermediate 16 (100 mg, 0.311 mmol), HATU (CARBOSYNTH, 142 mg, 0.373 mmol), N,N-diisopropylethylamine (FLUKA, 0.163 mL, 0.934 mmol) and 4-methoxybenzylamine (ALDRICH, 51.2 mg, 0.373 mmol) as starting reactants. The title compound (66.9 mg, 0.152 mmol, 49%) was obtained as a white solid. ¹H NMR (400 MHz, CDCl₃) δ ppm: 7.44 (s, 1H), 7.35 (d, 2H), 7.24-7.21 (m, 4H), 6.88 (d, 2H), 6.66-6.38 (m, 2H), 5.75-5.72 (m, 1H), 4.60-4.46 (m, 4H), 3.80 (s, 3H), 2.67 (q, 2H), 2.43-2.38 (m, 2H), 1.25 (t, 3H); [ES+MS] m/z 441 (M+H)⁺.

### Example 33

### Cis-7-(difluoromethyl)-5-(4-ethylphenyl)-N-(4-methylbenzyl)-4,5,6,7-tetrahydropyrazolo[1,5-a]pyrimidine-3-carboxamide

The title compound was prepared by a method analogous to that described for Example 1. Intermediate 16 (100 mg, 0.311 mmol), HATU (CARBOSYNTH, 142 mg, 0.373 mmol), N,N-diisopropylethylamine (FLUKA, 0.163 mL, 0.934 mmol) and 4-methylbenzylamine (ALDRICH, 45.3 mg, 0.373 mmol) as starting reactants. The title compound (45.7 mg, 0.108 mmol, 35%) was obtained as a white solid. ¹H NMR (400 MHz, CDCl₃) δ ppm: 7.44 (s, 1H), 7.35 (d, 2H), 7.23-7.21 (m, 4H), 7.15 (d, 2H), 6.65-6.38 (m, 2H), 5.76-5.74 (m, 1H), 4.56-4.49 (m, 4H), 2.67 (q, 2H), 2.43-2.38 (m, 2H), 2.34 (s, 3H), 1.25 (t, 3H); [ES+MS] m/z 423 (M+H)⁺.

### Example 34

### Cis-N-(benzo[d][1,3]dioxol-5-ylmethyl)-7-(difluoromethyl)-5-(4-ethylphenyl)-4,5,6,7-tetrahydropyrazolo[1,5-a]pyrimidine-3-carboxamide

The title compound was prepared by a method analogous to that described for Example 1. Intermediate 16 (100 mg, 0.311 mmol), HATU (CARBOSYNTH, 142 mg, 0.373 mmol), N,N-diisopropylethylamine (FLUKA, 0.163 mL, 0.934 mmol) and piperonylamine (ALDRICH, 56.5 mg, 0.373 mmol) as starting reactants. The title compound (52.6 mg, 0.116 mmol, 37%) was obtained as a white solid. ¹H NMR (400 MHz, CDCl₃) δ ppm: 7.45 (s, 1H), 7.35 (d, 2H), 7.22 (d, 2H), 6.82-6.77 (m, 3H), 6.75-6.38 (m, 2H), 5.95 (s, 2H), 5.77-5.74 (m, 1H), 4.60-4.54 (m, 2H), 4.49-4.39 (m, 2H), 2.67 (q, 2H), 2.43-2.38 (m, 2H), 1.25 (t, 3H); [ES+MS] m/z 455 (M+H)⁺.

### Example 35

### Cis-7-(tert-butyl)-5-(4-ethylphenyl)-N-(4-methoxybenzyl)-4,5,6,7-tetrahydropyrazolo[1,5-a]pyrimidine-3-carboxamide

The title compound was prepared by a method analogous to that described for Example 1. Intermediate 20 (85 mg, 0.260 mmol), HATU (CARBOSYNTH, 118 mg, 0.312 mmol), N,N-diisopropylethylamine (FLUKA, 0.136 mL, 0.779 mmol) and 4-methoxybenzylamine (ALDRICH, 46.3 mg, 0.337 mmol) as starting reactants. The title compound (95 mg, 0.213 mmol, 82%) was obtained as a white solid. ¹H NMR (400 MHz, CDCl₃) δ ppm: 7.39 (s, 1H), 7.36 (d, 2H), 7.26 (d, 2H), 7.21 (d, 2H), 6.87 (d, 2H), 6.52 (bs, 1H), 5.74-5.71 (m, 1H), 4.47 (d, 2H), 4.40 (dd, 1H), 4.06 (dd, 1H), 3.80 (s, 3H), 2.67 (q, 2H), 2.26-2.21 (m, 1H), 2.11-2.02 (m, 1H), 1.25 (t, 3H), 1.11 (s, 9H); [ES+MS] m/z 447 (M+H)⁺.

### Example 36

### Cis-N-(benzo[d][1,3]-dioxol-5-ylmethyl)-7-(tert-butyl)-5-(4-ethylphenyl)-4,5,6,7-tetrahydropyrazolo[1,5-a]pyrimidine-3-carboxamide

The title compound was prepared by a method analogous to that described for Example 1. Intermediate 20 (85 mg, 0.260 mmol), HATU (CARBOSYNTH, 118 mg, 0.312 mmol), N,N-diisopropylethylamine (FLUKA, 0.136 mL, 0.779 mmol) and piperonylamine (ALDRICH, 51.0 mg, 0.337 mmol) as starting reactants. The title compound (97 mg, 0.211 mmol, 81%) was obtained as a white solid. ¹H NMR (400 MHz, DMSO-d₆) δ ppm: 8.28-8.25 (m, 1H), 7.75 (s, 1H), 7.35 (d, 2H), 7.23 (d, 2H), 7.21 (d, 2H), 6.82 (d, 2H), 6.73 (bs, 1H), 6.35-6.33 (m, 1H), 5.95 (s, 2H), 4.42 (dd, 1H), 4.25 (d, 2H), 4.04 (dd, 1H), 2.59 (q, 2H), 2.18-2.13 (m, 1H), 1.93-1.87 (m, 1H), 1.17 (t, 3H), 1.05 (s, 9H); [ES+MS] m/z 461 (M+H)⁺.

The following compounds were purchased as indicated:

| Example | Name | Purchased from |
|---|---|---|
| Example | N-(benzo[d][1,3]dioxol-5-ylmethyl)-5-(4-ethylphenyl)-7- | Interbioscreen |
| 37 | (trifluoromethyl)-4,5,6,7-tetrahydropyrazolo[1,5- | Ltd. |
| | a]pyrimidine-3-carboxamide | |
| Example | N-(benzo[d][1,3]dioxol-5-ylmethyl)-5-(p-tolyl)-7- | ChemBridge |
| 38 | (trifluoromethyl)-4,5,6,7-tetrahydropyrazolo[1,5- | Corporation |
| | a]pyrimidine-3-carboxamide | |
| Example | N-(4-methoxybenzyl)-5-(p-tolyl)-7-(trifluoromethyl)- | AsinEx Limited |
| 39 | 4,5,6,7-tetrahydropyrazolo[1,5-a]pyrimidine-3- | |
| | carboxamide | |
| Example | 5-(4-ethylphenyl)-N-(thiophen-2-ylmethyl)-7- | Interbioscreen |
| 40 | (trifluoromethyl)-4,5,6,7-tetrahydropyrazolo[1,5- | Ltd. |
| | a]pyrimidine-3-carboxamide | |
| Example | N-(benzo[d][1,3]dioxol-5-ylmethyl)-5-(4- | Interbioscreen |
| 41 | methoxyphenyl)-7-(trifluoromethyl)-4,5,6,7- | Ltd. |
| | tetrahydropyrazolo[1,5-a]pyrimidine-3-carboxamide | |

The following compounds were prepared according to procedures analogous to those detailed above:

| Example | Name | MS |
|---|---|---|
| Example | Cis-5-(4-bromophenyl)-N-(4-methoxybenzyl)-7-(trifluoromethyl)- | [ES+ MS] |
| 42 | 4,5,6,7-tetrahydropyrazolo[1,5-a]pyrimidine-3-carboxamide | m/z |
| | | 509 (M + H)⁺. |
| Example | Cis-5-(p-tolyl)-7-(trifluoromethyl)-N-(2-(trifluoromethyl)benzyl)- | [ES+ MS] |
| 43 | 4,5,6,7-tetrahydropyrazolo[1,5-a]pyrimidine-3-carboxamide | m/z |
| | | 482 (M + H)⁺. |
| Example | Cis-5-(4-ethylphenyl)-7-(trifluoromethyl)-N-(2- | [ES+ MS] |
| 44 | (trifluoromethyl)benzyl)-4,5,6,7-tetrahydropyrazolo[1,5- | m/z |
| | a]pyrimidine-3-carboxamide | 496 (M + H)⁺. |
| Example | Cis-5-(4-ethylphenyl)-N-(2-methoxybenzyl)-7-(trifluoromethyl)- | [ES+ MS] |
| 45 | 4,5,6,7-tetrahydropyrazolo[1,5-a]pyrimidine-3-carboxamide | m/z |
| | | 458 (M + H)⁺. |
| Example | Cis-N-(2,4-dimethoxybenzyl)-5-(4-ethylphenyl)-7- | [ES+ MS] |
| 46 | (trifluoromethyl)-4,5,6,7-tetrahydropyrazolo[1,5-a]pyrimidine-3- | m/z |
| | carboxamide | 488 (M + H)⁺. |
| Example | Cis-N-(3,4-dimethoxybenzyl)-5-(4-ethylphenyl)-7- | [ES+ MS] |
| 47 | (trifluoromethyl)-4,5,6,7-tetrahydropyrazolo[1,5-a]pyrimidine-3- | m/z |
| | carboxamide | 488 (M + H)⁺. |
| Example | Cis-5-(4-ethylphenyl)-N-(pyridin-4-ylmethyl)-7-(trifluoromethyl)- | [ES+ MS] |
| 48 | 4,5,6,7-tetrahydropyrazolo[1,5-a]pyrimidine-3-carboxamide | m/z |
| | | 429 (M + H)⁺. |
| Example | Cis-N-(4-(dimethylamino)benzyl)-5-(p-tolyl)-7-(trifluoromethyl)- | [ES+ MS] |
| 49 | 4,5,6,7-tetrahydropyrazolo[1,5-a]pyrimidine-3-carboxamide | m/z |
| | | 457 (M + H)⁺. |
| Example | Cis-N-((2,3-dihydrobenzo[b][1,4]dioxin-6-yl)methyl)-5-(p-tolyl)-7- | [ES+ MS] |
| 50 | (trifluoromethyl)-4,5,6,7-tetrahydropyrazolo[1,5-a]pyrimidine-3- | m/z |
| | carboxamide | 472 (M + H)⁺. |
| Example | Cis-5-(4-ethylphenyl)-N-(2-furanylmethyl)-7-(trifluoromethyl)- | [ES+ MS] |
| 7b | 4,5,6,7-tetrahydropyrazolo[1,5-a]pyrimidine-3-carboxamide | m/z |
| | | 419 (M + H)⁺. |
| Example | Cis-5-(4-ethylphenyl)-N-(4-methoxybenzyl)-7-(trifluoromethyl)- | [ES+ MS] |
| 1b | 4,5,6,7-tetrahydropyrazolo[1,5-a]pyrimidine-3-carboxamide | m/z |
| | | 459 (M + H)⁺. |

The following compounds were purchased as indicated:

| | | Purchased |
|---|---|---|
| Example | Name | from |
| Example 51 | 7-(difluoromethyl)-N-(4-methylbenzyl)-5-(p- | AsinEx |
| | tolyl)-4,5,6,7-tetrahydropyrazolo[1,5- | Limited |
| | a]pyrimidine-3-carboxamide | |
| Example 52 | N-(2-chlorobenzyl)-5-(p-tolyl)-7- | ChemBridge |
| | (trifluoromethyl)-4,5,6,7- | Corporation |
| | tetrahydropyrazolo[1,5-a]pyrimidine-3- | |
| | carboxamide | |
| Example 53 | N-benzyl-5-(4-methoxyphenyl)-7- | AsinEx |
| | (trifluoromethyl)-4,5,6,7- | Limited |
| | tetrahydropyrazolo[1,5-a]pyrimidine-3- | |
| | carboxamide | |
| Example 54 | N-(4-methoxybenzyl)-5-(4-methoxyphenyl)- | Bio Specs |
| | 7-(trifluoromethyl)-4,5,6,7- | |
| | tetrahydropyrazolo[1,5-a]pyrimidine-3- | |
| | carboxamide | |
| Example 55 | 7-(difluoromethyl)-5-(p-tolyl)-N-(2- | AsinEx |
| | (trifluoromethyl)benzyl)-4,5,6,7- | Limited |
| | tetrahydropyrazolo[1,5-a]pyrimidine-3- | |
| | carboxamide | |

Comparative Examples 56 and 57, which are prior art compounds (Compounds 17f and 17e, respectively in: Tuberculosis, 2009, 89, 354-363) were purchased from the source shown in the table below, and were also prepared according to procedures analogous to those detailed above.

| | | Source where purchased; |
|---|---|---|
| Example | Name | MS where synthesized |
| Comparative | Cis-N-(furan-2-ylmethyl)-5-(p-tolyl)-7- | Purchased from |
| Example 56 | (trifluoromethyl)-4,5,6,7- | ChemDiv.; |
| | tetrahydropyrazolo[1,5-a]pyrimidine-3- | [ES+ MS] m/z 405 (M + H)⁺. |
| | carboxamide* | |
| Comparative | 5-(4-bromophenyl)-N-(furan-2-ylmethyl)-7- | Purchased from |
| Example 57 | (trifluoromethyl)-4,5,6,7- | Interbioscreen Ltd.; |
| | tetrahydropyrazolo[1,5-a]pyrimidine-3- | [ES+ MS] m/z 469 (M + H)⁺. |
| | carboxamide** | |

*Example 56: stereochemistry is cis for both synthesized (Ex 56′) and purchased (Ex 56″) compound.
**Example 57: stereochemistry is cis for synthesized (Ex 57′), and unknown for purchased (Ex 57″) compound.

### Example 58

### Cis-5-(4-ethylphenyl)-N-(4-fluorobenzyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (100 mg, 0.295 mmol), HATU (CARBOSYNTH, 134 mg, 0.354 mmol), N,N-diisopropylethylamine (FLUKA, 0.206 mL, 1.179 mmol) and 4-fluorobenzylamine Hydrochloride (ALDRICH, 57 mg, 0.354 mmol) as starting reactants. The title compound (64.5 mg, 0.137 mmol, 46.6%) was obtained as a pale yellow solid. ¹H NMR (400 MHz, CDCl₃) δ ppm: 7.52 (s, 1H), 7.35 (d, 2H), 7.31-7.27 (m, 2H), 7.23 (d, 2H), 7.05-6.99 (m, 2H), 6.65 (bs, 1H), 5.85-5.82 (m, 1H), 4.88-4.79 (m, 1H), 4.55-4.50 (m, 3H), 2.67 (q, 2H), 2.55-2.50 (m, 1H), 2.42-2.33 (m, 1H), 1.24 (t, 3H). [ES+MS] m/z 447 (M+H)⁺.

### Example 59

### Cis-N-[(2,4-difluorophenyl)methyl]-5-(4-ethylphenyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (100 mg, 0.295 mmol), HATU (CARBOSYNTH, 168 mg, 0.442 mmol), N,N-diisopropylethylamine (FLUKA, 0.154 mL, 0.884 mmol) and (2,4-difluorophenyl)methanamine (ALDRICH, 0.126 mL, 0.59 mmol) as starting reactants. The title compound (18.0 mg, 0.037 mmol, 12.5%) was obtained as a white solid. ¹H NMR (400 MHz, CDCl₃) δ ppm: 7.53 (s, 1H), 7.38-7.31 (m, 3H), 7.23 (d, 2H), 6.87-6.78 (m, 2H), 6.61 (bs, 1H), 5.87-5.92 (m, 1H), 4.89-4.79 (m, 1H), 4.60-4.48 (m, 3H), 2.67 (q, 2H), 2.56-2.49 (m, 1H), 2.42-2.31 (m, 1H), 1.25 (t, 3H). [ES+MS] m/z 465 (M+H)⁺.

### Example 60

### Cis-5-(4-ethylphenyl)-N-(3-fluorobenzyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (75 mg, 0.221 mmol), HATU (CARBOSYNTH, 126 mg, 0.332 mmol), N,N-diisopropylethylamine (FLUKA, 0.116 mL, 0.663 mmol) and (3-fluorophenyl)methanamine (ALDRICH, 0.038 mL, 0.333 mmol) as starting reactants. The title compound (84.0 mg, 0.179 mmol, 81%) was obtained as a white solid. ¹H NMR (400 MHz, CDCl₃) δ ppm: 7.55 (s, 1H), 7.36-7.26 (m, 3H), 7.23 (d, 2H), 7.09 (d, 1H), 7.03 (d, 1H), 6.97 (t, 1H), 6.65 (bs, 1H), 5.93-5.87 (m, 1H), 4.89-4.80 (m, 1H), 4.60-4.48 (m, 3H), 2.67 (q, 2H), 2.57-2.49 (m, 1H), 2.44-2.33 (m, 1H), 1.25 (t, 3H). [ES+MS] m/z 447 (M+H)⁺.

### Example 61

### Cis-5-(4-ethylphenyl)-N-[(1,5-methyl-2-thienyl)methyl]-7-(trifluoromethyl)-4,5,6,7-tetrahydropyrazolo[1,5-a]pyrimidine-3-carboxamide

The title compound was prepared by a method analogous to that described for Example 1. Intermediate 3 (100 mg, 0.295 mmol), HATU (CARBOSYNTH, 134 mg, 0.354 mmol), N,N-diisopropylethylamine (FLUKA, 0.206 mL, 1.179 mmol) and (5-methylthien-2-yl)methylamine hydrochloride (MAYBRIDGE, 58 mg, 0.354 mmol) as starting reactants. The title compound (95 mg, 0.191 mmol, 65%) was obtained. ¹H NMR (400 MHz, CDCl₃) δ ppm: 7.51 (s, 1H), 7.37-7.35 (d, 2H), 7.25-7.23 (d, 2H), 6.79-6.78 (m, 1H), 6.66 (bs, 1H), 6.60-6.59 (m, 1H), 5.83-5.80 (m, 1H), 4.88-4.80 (m, 1H), 4.69-4.52 (m, 3H), 2.68 (q, 2H), 2.56-2.50 (m, 1H), 2.45 (s, 3H), 2.42-2.33 (m, 1H), 1.26 (t, 3H). [ES+MS] m/z 449 (M+H)⁺.

### Example 62

### Cis-5-(4-ethylphenyl)-N-(2-fluorobenzyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (75 mg, 0.221 mmol), HATU (CARBOSYNTH, 126 mg, 0.332 mmol), N,N-diisopropylethylamine (FLUKA, 0.116 mL, 0.663 mmol) and (2-fluorophenyl)methanamine (ALDRICH, 38 μL, 0.332 mmol) as starting reactants. The title compound (40 mg, 0.085 mmol, 38%) was obtained as a white solid. ¹H NMR (400 MHz, CDCl₃) δ ppm: 7.54 (s, 1H), 7.39-7.34 (m, 3H), 7.29-7.22 (m, 3H), 7.13-7.04 (m, 2H), 6.64 (bs, 1H), 5.95-5.92 (m, 1H), 4.88-4.80 (m, 1H), 4.64-4.51 (m, 3H), 2.67 (q, 2H), 2.55-2.50 (m, 1H), 2.42-2.33 (m, 1H), 1.25 (t, 3H). [ES+MS] m/z 447 (M+H)⁺.

### Example 63

### Cis-5-(4-ethylphenyl)-N-(4-fluoro-3-methylbenzyl)-7-(trifluoromethyl)-4,5,6,7-tetrahydropyrazolo[1,5-a]pyrimidine-3-carboxamide

The title compound was prepared by a method analogous to that described for Example 1 Intermediate 3 (100 mg, 0.295 mmol), HATU (CARBOSYNTH, 134 mg, 0.354 mmol), N,N-diisopropylethylamine (FLUKA, 0.206 mL, 1.179 mmol) and 4-fluoro-3-methyl benzylamine (MATRIX, 49 mg, 0.354 mmol) as starting reactants. The title compound (75 mg, 0.155 mmol, 52.5%) was obtained as a white solid. ¹H NMR (400 MHz, CDCl₃) δ ppm: 7.52 (s, 1H), 7.35 (d, 2H), 7.22 (d, 2H), 7.14-7.07 (m, 2H), 6.98-6.93 (m, 1H), 6.66 (bs, 1H), 5.82-5.79 (m, 1H), 4.88-4.79 (m, 1H), 4.55-4.51 (m, 1H), 4.47-4.46 (m, 2H), 2.67 (q, 2H), 2.56-2.49 (m, 1H), 2.42-2.33 (m, 1H), 2.25 (m, 3H), 1.24 (t, 3H). [ES+MS] m/z 461 (M+H)⁺.

### Example 64

### Cis-5-(4-ethylphenyl)-N-(3-fluoro-4-methylbenzyl)-7-(trifluoromethyl)-4,5,6,7-tetrahydropyrazolo[1,5-a]pyrimidine-3-carboxamide

The title compound was prepared by a method analogous to that described for Example 1 Intermediate 3 (100 mg, 0.295 mmol), HATU (CARBOSYNTH, 134 mg, 0.354 mmol), N,N-diisopropylethylamine (FLUKA, 0.206 mL, 1.179 mmol) and 3-fluoro-4-methyl benzylamine (MATRIX, 49 mg, 0.354 mmol) as starting reactants. The title compound (75.3 mg, 0.155 mmol, 52.7%) was obtained as a pale yellow solid. ¹H NMR (400 MHz, CDCl₃) δ ppm: 7.52 (s, 1H), 7.35 (d, 2H), 7.22 (d, 2H), 7.15-7.11 (m, 1H), 6.99-6.96 (m, 2H), 6.65 (bs, 1H), 5.85-5.82 (m, 1H), 4.88-4.80 (m, 1H), 4.55-4.44 (m, 3H), 2.66 (q, 2H), 2.56-2.49 (m, 1H), 2.42-2.33 (m, 1H), 2.25 (m, 3H), 1.24 (t, 3H). [ES+MS] m/z 461 (M+H)⁺.

### Example 65

### Cis-N-(3,4-dimethylbenzyl)-5-(4-ethylphenyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (40 mg, 0.118 mmol), HATU (CARBOSYNTH, 67.2 mg, 0.177 mmol), N,N-diisopropylethylamine (FLUKA, 0.062 mL, 0.355 mmol) and 3,4-dimethylphenyl)methanamine (Trans World Chemicals Inc, 24 mg, 0.178 mmol) as starting reactants. The title compound (17 mg, 0.037 mmol, 32%) was obtained as a white solid. ¹H NMR (400 MHz, CDCl₃) δ ppm: 7.51 (s, 1H), 7.36 (d, 2H), 7.23 (d, 2H), 7.14-7.03 (m, 3H), 6.69 (bs, 1H), 5.81 (bt, 1H), 4.89-4.78 (m, 1H), 4.53 (dd, 1H), 4.48 (d, 2H), 2.72-2.63 (m, 2H), 2.57-2.48 (m, 1H), 2.44-2.31 (m, 1H), 2.26 (s, 3H), 2.25 (s, 3H), 1.26 (t, 3H). [ES+MS] m/z 457 (M+H)⁺.

### Example 66

### Cis-5-(4-ethylphenyl)-7-isopropyl-N-(4-methylbenzyl)-4,5,6,7-tetrahydropyrazolo[1,5-a]pyrimidine-3-carboxamide

The title compound was prepared by a method analogous to that described for Example 1. Intermediate 29 (80 mg, 0.255 mmol), HATU (CARBOSYNTH, 116 mg, 0.306 mmol), N,N-diisopropylethylamine (FLUKA, 0.134 mL, 0.766 mmol) and 4-Methylbenzylamine (ALDRICH, 44.2 mg, 0.332 mmol) as starting reactants. The title compound (70.6 mg, 0.169 mmol, 66%) was obtained as a white solid. ¹H NMR (400 MHz, CDCl₃) δ ppm: 7.42 (s, 1H), 7.36 (d, 2H), 7.24-7.19 (m, 4H), 7.15 (d, 2H), 6.43 (bs, 1H), 5.75-5.73 (m, 1H), 4.55-4.45 (m, 3H), 4.23-4.18 (m, 1H), 2.94-2.86 (m, 1H), 2.67 (q, 2H), 2.34 (s, 3H), 2.14-1.98 (m, 2H), 1.25 (t, 3H) 1.01 (d, 3H), 0.78 (d, 3H). [ES+MS] m/z 417 (M+H)⁺.

### Example 67

### Cis-N-(4-chlorobenzyl)-5-(4-ethylphenyl)-7-(trifluoromethyl)-4,5,6,7-tetrahydropyrazolo[1,5-a]pyrimidine-3-carboxamide

The title compound was prepared by a method analogous to that described for Example 1 Intermediate 3 (70 mg, 0.206 mmol), HATU (CARBOSYNTH, 94 mg, 0.248 mmol), N,N-diisopropylethylamine (FLUKA, 0.108 mL, 0.619 mmol) and 4-chloro benzylamine (ALDRICH, 35 mg, 0.248 mmol) as starting reactants. The title compound (43 mg, 0.088 mmol, 42.8%) was obtained as a white solid. ¹H NMR (400 MHz, CDCl₃) δ ppm: 7.53 (s, 1H), 7.35-7.21 (m, 8H), 6.64 (bs, 1H), 5.87-5.84 (m, 1H), 4.87-4.81 (m, 1H), 4.55-4.50 (m, 3H), 2.66 (q, 2H), 2.56-2.50 (m, 1H), 2.42-2.33 (m, 1H), 1.24 (t, 3H). [ES+MS] m/z 463 (M+H)⁺.

### Example 68

### Cis-7-(1,1-difluoroethyl)-5-(4-ethylphenyl)-N-(4-methylbenzyl)-4,5,6,7-tetrahydropyrazolo[1,5-a]pyrimidine-3-carboxamide

The title compound was prepared by a method analogous to that described for Example 1. Intermediate 24 (100 mg, 0.298 mmol), HATU (CARBOSYNTH, 136 mg, 0.358 mmol), N,N-diisopropylethylamine (FLUKA, 0.156 mL, 0.895 mmol) and 4-Methylbenzylamine (ALDRICH, 43.4 mg, 0.358 mmol) as starting reactants. The title compound (92 mg, 0.210 mmol, 70%) was obtained as a white solid. ¹H NMR (400 MHz, CDCl₃) δ ppm: 7.46 (s, 1H), 7.35 (d, 2H), 7.23-7.21 (m, 4H), 7.15 (d, 2H), 6.61 (bs, 1H), 5.79-5.76 (m, 1H), 4.64-4.59 (m, 1H), 4.53-4.49 (m, 3H), 2.67 (q, 2H), 2.51-2.46 (m, 1H), 2.34 (s, 3H), 2.25-2.15 (m, 1H), 1.76 (t, 3H), 1.25 (t, 3H). [ES+MS] m/z 439 (M+H)⁺.

### Example 69

### Cis-5-(4-ethylphenyl)-N-((5-fluoropyridin-2-yl)methyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (200 mg, 0.589 mmol), HATU (CARBOSYNTH, 269 mg, 0.707 mmol), N,N-diisopropylethylamine (FLUKA, 0.618 mL, 3.536 mmol) and (5-fluoropyridin-2-yl)methanamine, 2 Hydrochloride (prepared as described in WO2005/66126) (153 mg, 0.766 mmol) as starting reactants. The title compound (221 mg, 0.494 mmol, 80%) was obtained as an off-white solid. ¹H NMR (400 MHz, CDCl₃) δ ppm: 8.42 (d, 1H), 7.63 (s, 1H), 7.42-7.31 (m, 4H), 7.23 (d, 2H), 6.60 (m, 2H), 4.85 (m, 1H), 4.63 (d, 2H), 4.55-4.52 (m, 1H), 2.67 (q, 2H), 2.56-2.50 (m, 1H), 2.42-2.33 (m, 1H), 1.25 (t, 3H) [ES+MS] m/z 448 (M+H)⁺.

### Example 70

### Cis N-((3,5-difluoropyridin-2-yl)methyl)-5-(4-ethylphenyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (100 mg, 0.295 mmol), HATU (CARBOSYNTH, 168 mg, 0.442 mmol), N,N-diisopropylethylamine (FLUKA, 0.308 mL, 1.764 mmol) and (3,5-difluoropyridin-2-yl)methanamine hydrochloride (prepared as described in WO2005/66126 83 mg, 0.460 mmol) as starting reactants. The title compound (30.5 mg, 0.066 mmol, 22%) was obtained as a beige solid. ¹H NMR (400 MHz, CDCl₃) δ ppm: 8.32 (s, 1H), 7.65 (s, 1H), 7.35 (d, 2H), 7.26-7.16 (m, 3H), 6.70 (bt, 1H), 6.63 (bs, 1H), 4.90-4.79 (m, 1H), 4.70 (d, 2H), 4.53 (dd, 1H), 2.72-2.63 (m, 2H), 2.57-2.48 (m, 1H), 2.43-2.31 (m, 1H), 1.25 (t, 3H). [ES+MS] m/z 466 (M+H)⁺.

### Example 71

### Cis-5-(4-ethylphenyl)-7-isopropyl-N-(4-methoxybenzyl)-4,5,6,7-tetrahydropyrazolo[1,5-a]pyrimidine-3-carboxamide

The title compound was prepared by a method analogous to that described for Example 1. Intermediate 29 (50 mg, 0.160 mmol), HATU (CARBOSYNTH, 72.8 mg, 0.191 mmol), N,N-diisopropylethylamine (FLUKA, 0.084 mL, 0.479 mmol) and 4-Methoxylbenzylamine (ALDRICH, 28.5 mg, 0.207 mmol) as starting reactants. The title compound (62.4 mg, 0.144 mmol, 90%) was obtained as a white solid. ¹H NMR (400 MHz, CDCl₃) δ ppm: 7.43 (s, 1H), 7.36 (d, 2H), 7.27-7.19 (m, 4H), 6.87 (d, 2H), 6.43 (bs, 1H), 5.75-5.72 (m, 1H), 4.52-4.46 (m, 3H), 4.23-4.18 (m, 1H), 3.80 (s, 3H), 2.92-2.86 (m, 1H), 2.67 (q, 2H), 2.14-1.98 (m, 2H), 1.25 (t, 3H), 1.01 (d, 3H), 0.78 (d, 3H). [ES+MS] m/z 433 (M+H)⁺.

### Example 72

### Cis-N-((1,5-dimethyl-1H-pyrrol-2-yl)methyl)-5-(4-ethylphenyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (50 mg, 0.147 mmol), HATU (CARBOSYNTH, 84 mg, 0.221 mmol), N,N-diisopropylethylamine (FLUKA, 0.097 mL, 0.555 mmol) and (1,5-dimethyl-1H-pyrrol-2-yl)methanamine (MAYBRIDGE, 27.5 mg, 0.221 mmol) as starting reactants. The title compound (40 mg, 0.090 mmol, 61%) was obtained as a brown solid. ¹H NMR (400 MHz, CDCl₃) δ ppm: 7.50 (s, 1H), 7.37 (d, 2H), 7.24 (d, 2H), 6.67 (bs, 1H), 6.04 (bs, 1H), 5.58 (bs, 1H), 5.61 (bt, 1H), 4.90-4.78 (m, 1H), 4.61-4.44 (m, 3H), 3.44 (s, 3H), 2.72-2.63 (m, 2H), 2.58-2.50 (m, 1H), 2.43-2.32 (m, 1H), 2.22 (s, 3H), 1.25 (t, 3H). [ES+MS] m/z 446 (M−H+).

### Example 73

### Cis-N-(2-chlorobenzyl)-5-(4-ethylphenyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (100 mg, 0.295 mmol), HATU (CARBOSYNTH, 134 mg, 0.354 mmol), N,N-diisopropylethylamine (FLUKA, 0.154 mL, 0.884 mmol) and (2-chlorophenyl)methanamine (ALDRICH, 50.1 mg, 0.354 mmol) as starting reactants. The title compound (100 mg, 0.216 mmol, 73%) was obtained as beige solid. ¹H NMR (400 MHz, CDCl₃) δ ppm: 7.55 (s, 1H), 7.43-7.34 (m, 4H), 7.25-7.22 (m, 4H), 6.63 (bs, 1H), 6.01-5.98 (m, 1H), 4.88-4.80 (m, 1H), 4.69-4.58 (m, 2H), 4.54-4.51 (m, 1H), 2.67 (q, 2H), 2.56-2.50 (m, 1H), 2.42-2.33 (m, 1H), 1.25 (t, 3H). [ES+MS] m/z 463 (M+H)⁺.

### Example 74

### Cis5-(4-ethylphenyl)-N-((6-methoxypyridin-3-yl)methyl)-7-(trifluoromethyl)-4,5,6,7-tetrahydropyrazolo[1,5-a]pyrimidine-3-carboxamide

The title compound was prepared by a method analogous to that described for Example 1 Intermediate 3 (280 mg, 0.825 mmol), HATU (CARBOSYNTH, 377 mg, 0.990 mmol), N,N-diisopropylethylamine (FLUKA, 0.432 mL, 2.476 mmol) and (6-methoxypyridin-3-yl)methanamine (Syngene International Pvt. Ltd., India 0.089 ml, 0.990 mmol) as starting reactants. The title compound (7 mg, 0.014 mmol, 1.7%) was obtained as a yellow solid. ¹H NMR (400 MHz, CDCl₃) δ ppm: 8.11 (d, 1H), 7.57 (dd, 1H), 7.50 (s, 1H), 7.35 (d, 2H), 7.23 (d, 2H), 6.72 (d, 1H), 6.64 (bs, 1H), 5.80-5.76 (m, 1H), 4.86-4.80 (m, 1H), 4.55-4.52 (m, 1H), 4.47-4.46 (m, 2H), 3.92 (s, 3H), 2.67 (q, 2H), 2.55-2.49 (m, 1H), 2.42-2.32 (m, 1H), 1.25 (t, 3H). [ES+MS] m/z 460 (M+H)⁺.

### Example 75

### Cis-N-((5-chloropyridin-2-yl)methyl)-5-(4-ethylphenyl)-7-(trifluoromethyl)-4,5,6,7-tetrahydropyrazolo[1,5-a]pyrimidine-3-carboxamide

The title compound was prepared by a method analogous to that described for Example 1 Intermediate 3 (55 mg, 0.162 mmol), HATU (CARBOSYNTH, 74 mg, 0.195 mmol), N,N-diisopropylethylamine (FLUKA, 0.085 mL, 0.486 mmol) and 5-chloropyridin-2-yl)methanamine (BETAPHARMA, 23 mg, 0.162 mmol) as starting reactants. The title compound (11.5 mg, 0.024 mmol, 14.5%) was obtained as a solid. ¹H NMR (400 MHz, CDCl₃) δ ppm: 8.35 (d, 1H), 7.65 (dd, 1H), 7.54 (s, 1H), 7.34 (d, 2H), 7.29 (d, 1H), 7.23 (d, 2H), 6.60 (bs, 1H), 5.94-5.91 (m, 1H), 4.87-4.81 (m, 1H), 4.56-4.48 (m, 3H), 2.67 (q, 2H), 2.56-2.50 (m, 1H), 2.42-2.33 (m, 1H), 1.24 (t, 3H). [ES+MS] m/z 464 (M+H)⁺.

### Example 76

### Cis-N-((6-chloropyridin-3-yl)methyl)-5-(4-ethylphenyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (50 mg, 0.147 mmol), HATU (CARBOSYNTH, 84 mg, 0.221 mmol), N,N-diisopropylethylamine (FLUKA, 0.097 mL, 0.555 mmol) and (6-chloropyridin-3-yl)methanamine (ALDRICH, 31.5 mg, 0.221 mmol) as starting reactants. The title compound (40 mg, 0.086 mmol, 58%) was obtained as a colourless oil. ¹H NMR (400 MHz, CDCl₃) δ ppm: 8.35 (d, 1H), 7.67 (dd, 1H), 7.58 (s, 1H), 7.35 (d, 2H), 7.29 (d, 1H), 7.24 (d, 2H), 6.61 (bs, 1H), 6.17 (bt, 1H), 4.90-4.79 (m, 1H), 4.58-4.46 (m, 3H), 2.72-2.63 (m, 2H), 2.58-2.50 (m, 1H), 2.43-2.32 (m, 1H), 1.25 (t, 3H). [ES+MS] m/z 464 (M−H+).

### Example 77

### Cis-5-(4-ethylphenyl)-N-((5-fluoro-6-methylpyridin-2-yl)methyl)-7-(trifluoromethyl)-4,5,6,7-tetrahydropyrazolo[1,5-a]pyrimidine-3-carboxamide

The title compound was prepared by a method analogous to that described for Example 1. Intermediate 3 (80 mg, 0.236 mmol), HATU (CARBOSYNTH, 134 mg, 0.354 mmol), N,N-diisopropylethylamine (FLUKA, 0.124 mL, 0.710 mmol) and (5-fluoro-6-methylpyridin-2-yl)methanamine (prepared as described in WO2005061497) (49.2 mg, 0.351 mmol) as starting reactants. The title compound (19 mg, 0.041 mmol, 17%) was obtained as a colourless oil. ¹H NMR (400 MHz, CDCl₃) δ ppm: 7.63 (s, 1H), 7.35 (d, 2H), 7.30 (d, 1H), 7.25 (d, 2H), 7.16-7.12 (m, 1), 6.65 (bt, 1H), 6.61 (bs, 1H), 4.80-4.90 (m, 1H), 4.59 (d, 2H), 4.53 (dd, 1H), 2.71-2.63 (m, 2H), 2.57-2.48 (m, 1H), 2.53 (s, 3H), 2.43-2.32 (m, 1H), 1.25 (t, 3H). [ES+MS] m/z 462 (M+H)⁺.

### Examples 78a and 78b

### Enantiomers a and b of cis-5-(4-ethylphenyl)-N-(3-methylbenzyl)-7-(trifluoromethyl)-4,5,6,7-tetrahydropyrazolo[1,5-a]pyrimidine-3-carboxamide

Cis-5-(4-ethylphenyl)-N-(3-methylbenzyl)-7-(trifluoromethyl)-4,5,6,7-tetrahydropyrazolo[1,5-a]pyrimidine-3-carboxamide was subjected to semi-preparative high performance liquid chromatography (HPLC) to afford the optically pure enantiomers Examples 78a (5R,7S)-5-(4-ethylphenyl)-N-(3-methylbenzyl)-7-(trifluoromethyl)-4,5,6,7-tetrahydropyrazolo[1,5-a]pyrimidine-3-carboxamide, >95% ee (13 minutes) and Examples 78b (5S,7R)-5-(4-ethylphenyl)-N-(3-methylbenzyl)-7-(trifluoromethyl)-4,5,6,7-tetrahydropyrazolo[1,5-a]pyrimidine-3-carboxamide, >95% ee (19 minutes). [Column: Chiralpack IC, 250×20 mm, temperature 25° C., mobile phase: hexane/ethanol 93/7, Flow rate: 18 ml/minute, detection wavelength: 254 nm, and injection of 70 mg.

### Examples 79a and 79b

### Enantiomers a and b of cis-7-(difluoromethyl)-5-(4-ethylphenyl)-N-(4-methoxybenzyl)-4,5,6,7-tetrahydropyrazolo[1,5-a]pyrimidine-3-carboxamide

Cis-7-(difluoromethyl)-5-(4-ethylphenyl)-N-(4-methoxybenzyl)-4,5,6,7-tetrahydropyrazolo[1,5-a]pyrimidine-3-carboxamide was subjected to semi-preparative high performance liquid chromatography (HPLC) to afford the optically pure enantiomers Examples 79a (5R,7S)-7-(difluoromethyl)-5-(4-ethylphenyl)-N-(4-methoxybenzyl)-4,5,6,7-tetrahydropyrazolo[1,5-a]pyrimidine-3-carboxamide, >95% ee (16 minutes) and Examples 79b (5S,7R)-7-(difluoromethyl)-5-(4-ethylphenyl)-N-(4-methoxybenzyl)-4,5,6,7-tetrahydropyrazolo[1,5-a]pyrimidine-3-carboxamide, >95% ee (Tiempo retencion). [Column: Chiralpack IC, 250×20 mm, temperature 25° C., mobile phase: hexane/ethanol 90/10, Flow rate: 18 ml/minute, detection wavelength: 254 nm, and injection of 50 mg.

### Examples 80a and 80b

### Enantiomers a and b of cis-5-(4-ethylphenyl)-N-(4-fluorobenzyl)-7-(trifluoromethyl)-4,5,6,7-tetrahydropyrazolo[1,5-a]pyrimidine-3-carboxamide

Cis-5-(4-ethylphenyl)-N-(4-fluorobenzyl)-7-(trifluoromethyl)-4,5,6,7-tetrahydropyrazolo[1,5-a]pyrimidine-3-carboxamide was subjected to semi-preparative high performance liquid chromatography (HPLC) to afford the optically pure enantiomers Example 80a (5R,7S)-5-(4-ethylphenyl)-N-(4-fluorobenzyl)-7-(trifluoromethyl)-4,5,6,7-tetrahydropyrazolo[1,5-a]pyrimidine-3-carboxamide, >95% ee (17 minutes) and Example 80b (5S,7R)-5-(4-ethylphenyl)-N-(4-fluorobenzyl)-7-(trifluoromethyl)-4,5,6,7-tetrahydropyrazolo[1,5-a]pyrimidine-3-carboxamide, >95% ee (28 minutes). [Column: Chiralpack IC, 250×20 mm, temperature 25° C., mobile phase: hexane/ethanol 95/5, Flow rate: 18 ml/minute, detection wavelength: 254 nm, and injection of 58 mg.

### Examples 81a and 81b

### Enantiomers a and b of cis-N-(3,4-difluorobenzyl)-5-(p-tolyl)-7-(trifluoromethyl)-4,5,6,7-tetrahydropyrazolo[1,5-a]pyrimidine-3-carboxamide

Cis-N-(3,4-difluorobenzyl)-5-(p-tolyl)-7-(trifluoromethyl)-4,5,6,7-tetrahydropyrazolo[1,5-a]pyrimidine-3-carboxamide was subjected to semi-preparative high performance liquid chromatography (HPLC) to afford the optically pure enantiomers Example 81a (5R,7S)—N-(3,4-difluorobenzyl)-5-(p-tolyl)-7-(trifluoromethyl)-4,5,6,7-tetrahydropyrazolo[1,5-a]pyrimidine-3-carboxamide, >95% ee (12 minutes) and Example 81b (5S,7R)—N-(3,4-difluorobenzyl)-5-(p-tolyl)-7-(trifluoromethyl)-4,5,6,7-tetrahydropyrazolo[1,5-a]pyrimidine-3-carboxamide, >95% ee (22 minutes). [Column: Chiralpack IC, 250×20 mm, temperature 25° C., mobile phase: hexane/ethanol 95/5, Flow rate: 18 ml/minute, detection wavelength: 254 nm, and injection of 59 mg.

### Examples 82a and 82b

### Enantiomers a and b of cis-N-(benzo[d][1,3]dioxol-5-ylmethyl)-7-(difluoromethyl)-5-(4-ethylphenyl)-4,5,6,7-tetrahydropyrazolo[1,5-a]pyrimidine-3-carboxamide

Cis-N-(benzo[d][1,3]dioxol-5-ylmethyl)-7-(difluoromethyl)-5-(4-ethylphenyl)-4,5,6,7-tetrahydropyrazolo[1,5-a]pyrimidine-3-carboxamide was subjected to semi-preparative high performance liquid chromatography (HPLC) to afford the optically pure enantiomers

Example 82a (5R,7S)—N-(benzo[d][1,3]dioxol-5-ylmethyl)-7-(difluoromethyl)-5-(4-ethylphenyl)-4,5,6,7-tetrahydropyrazolo[1,5-a]pyrimidine-3-carboxamide, >95% ee (12 minutes) and Example 82b (5S,7R)—N-(benzo[d][1,3]dioxol-5-ylmethyl)-7-(difluoromethyl)-5-(4-ethylphenyl)-4,5,6,7-tetrahydropyrazolo[1,5-a]pyrimidine-3-carboxamide, >95% ee (17 minutes). [Column: Chiralpack IC, 250×20 mm, temperature 25° C., mobile phase: hexane/ethanol 80/20, Flow rate: 18 ml/minute, detection wavelength: 254 nm, and injection of 40 mg.

### Examples 83a and 83b

### Enantiomers a and b of cis-5-(4-ethylphenyl)-N-(3-fluorobenzyl)-7-(trifluoromethyl)-4,5,6,7-tetrahydropyrazolo[1,5-a]pyrimidine-3-carboxamide

Cis-5-(4-ethylphenyl)-N-(3-fluorobenzyl)-7-(trifluoromethyl)-4,5,6,7-tetrahydropyrazolo[1,5-a]pyrimidine-3-carboxamide was subjected to semi-preparative high performance liquid chromatography (HPLC) to afford the optically pure enantiomers Example 83a (5R,7S)-5-(4-ethylphenyl)-N-(3-fluorobenzyl)-7-(trifluoromethyl)-4,5,6,7-tetrahydropyrazolo[1,5-a]pyrimidine-3-carboxamide, >95% ee (14 minutes) and Example 83b (5S,7R)-5-(4-ethylphenyl)-N-(3-fluorobenzyl)-7-(trifluoromethyl)-4,5,6,7-tetrahydropyrazolo[1,5-a]pyrimidine-3-carboxamide, >95% ee (24 minutes). [Column: Chiralpack IC, 250×20 mm, temperature 25° C., mobile phase: hexane/ethanol 95/5, Flow rate: 18 ml/minute, detection wavelength: 254 nm, and injection of 60 mg.

### Examples 84a and 84b

### Enantiomers a and b of cis-5-(4-ethylphenyl)-N-(3-fluoro-4-methylbenzyl)-7-(trifluoromethyl)-4,5,6,7-tetrahydropyrazolo[1,5-a]pyrimidine-3-carboxamide

Cis-5-(4-ethylphenyl)-N-(3-fluoro-4-methylbenzyl)-7-(trifluoromethyl)-4,5,6,7-tetrahydropyrazolo[1,5-a]pyrimidine-3-carboxamide was subjected to semi-preparative high performance liquid chromatography (HPLC) to afford the optically pure enantiomers Examples 84a (5R,7S)-5-(4-ethylphenyl)-N-(3-fluoro-4-methylbenzyl)-7-(trifluoromethyl)-4,5,6,7-tetrahydropyrazolo[1,5-a]pyrimidine-3-carboxamide, >95% ee (8 minutes) and Examples 84b (5S,7R)-5-(4-ethylphenyl)-N-(3-fluoro-4-methylbenzyl)-7-(trifluoromethyl)-4,5,6,7-tetrahydropyrazolo[1,5-a]pyrimidine-3-carboxamide, >95% ee (12 minutes). [Column: Chiralpack IC, 250×20 mm, temperature 25° C., mobile phase: hexane/ethanol 90/10, Flow rate: 18 ml/minute, detection wavelength: 254 nm, and injection of 60 mg.

### Examples 85a and 85b

### Enantiomers a and b of cis-N-(cyclohexylmethyl)-5-(4-ethylphenyl)-7-(trifluoromethyl)-4,5,6,7-tetrahydropyrazolo[1,5-a]pyrimidine-3-carboxamide

Cis-N-(cyclohexylmethyl)-5-(4-ethylphenyl)-7-(trifluoromethyl)-4,5,6,7-tetrahydropyrazolo[1,5-a]pyrimidine-3-carboxamide was subjected to semi-preparative high performance liquid chromatography (HPLC) to afford the optically pure enantiomers Examples 85a (5R,7S)—N-(cyclohexylmethyl)-5-(4-ethylphenyl)-7-(trifluoromethyl)-4,5,6,7-tetrahydropyrazolo[1,5-a]pyrimidine-3-carboxamide, >95% ee (11 minutes) and Examples 85b (5R,7S)—N-(cyclohexylmethyl)-5-(4-ethylphenyl)-7-(trifluoromethyl)-4,5,6,7-tetrahydropyrazolo[1,5-a]pyrimidine-3-carboxamide, >95% ee (17 minutes). [Column: Chiralpack IC, 250×20 mm, temperature 25° C., mobile phase: hexane/ethanol 95/5, Flow rate: 18 ml/minute, detection wavelength: 254 nm, and injection of 100 mg.

### Examples 86a and 86b

### Enantiomers a and b of cis-N-(benzo[d][1,3]dioxol-5-ylmethyl)-5-(4-bromophenyl)-7-(trifluoromethyl)-4,5,6,7-tetrahydropyrazolo[1,5-a]pyrimidine-3-carboxamide

Cis-N-(benzo[d][1,3]dioxol-5-ylmethyl)-5-(4-bromophenyl)-7-(trifluoromethyl)-4,5,6,7-tetrahydropyrazolo[1,5-a]pyrimidine-3-carboxamide was subjected to semi-preparative high performance liquid chromatography (HPLC) to afford the optically pure enantiomers 86a (5R,7S)—N-(benzo[d][1,3]dioxol-5-ylmethyl)-5-(4-bromophenyl)-7-(trifluoromethyl)-4,5,6,7-tetrahydropyrazolo[1,5-a]pyrimidine-3-carboxamide >95% ee (15 minutes) and 86b (5S,7R)—N-(benzo[d][1,3]dioxol-5-ylmethyl)-5-(4-bromophenyl)-7-(trifluoromethyl)-4,5,6,7-tetrahydropyrazolo[1,5-a]pyrimidine-3-carboxamide, >95% ee (25 minutes). [Column: Chiralpack IC, 250×20 mm, temperature 25° C., mobile phase: hexane/ethanol 90/10, Flow rate: 18 ml/minute, detection wavelength: 254 nm, and injection of 110 mg.

### Examples 87a and 87b

### Enantiomers a and b of cis-5-(4-ethylphenyl)-N-((5-methylthiophen-2-yl)methyl)-7-(trifluoromethyl)-4,5,6,7-tetrahydropyrazolo[1,5-a]pyrimidine-3-carboxamide

Cis-5-(4-ethylphenyl)-N-((5-methylthiophen-2-yl)methyl)-7-(trifluoromethyl)-4,5,6,7-tetrahydropyrazolo[1,5-a]pyrimidine-3-carboxamide was subjected to semi-preparative high performance liquid chromatography (HPLC) to afford the optically pure enantiomers Examples 87a (5R,7S)-5-(4-ethylphenyl)-N-((5-methylthiophen-2-yl)methyl)-7-(trifluoromethyl)-4,5,6,7-tetrahydropyrazolo[1,5-a]pyrimidine-3-carboxamide, >95% ee (9 minutes) and Examples 87b (5S,7R)-5-(4-ethylphenyl)-N-((5-methylthiophen-2-yl)methyl)-7-(trifluoromethyl)-4,5,6,7-tetrahydropyrazolo[1,5-a]pyrimidine-3-carboxamide, >95% ee (14 minutes). [Column: Chiralpack IC, 250×20 mm, temperature 25° C., mobile phase: hexane/ethanol 90/10, Flow rate: 18 ml/minute, detection wavelength: 254 nm, and injection of 60 mg.

### Examples 88a and 88b

### Enantiomers a and b of cis-5-(4-ethylphenyl)-N-(4-fluoro-3-methylbenzyl)-7-(trifluoromethyl)-4,5,6,7-tetrahydropyrazolo[1,5-a]pyrimidine-3-carboxamide

Cis-5-(4-ethylphenyl)-N-(4-fluoro-3-methylbenzyl)-7-(trifluoromethyl)-4,5,6,7-tetrahydropyrazolo[1,5-a]pyrimidine-3-carboxamide was subjected to semi-preparative high performance liquid chromatography (HPLC) to afford the optically pure enantiomers Examples 88a (5R,7S)-5-(4-ethylphenyl)-N-(4-fluoro-3-methylbenzyl)-7-(trifluoromethyl)-4,5,6,7-tetrahydropyrazolo[1,5-a]pyrimidine-3-carboxamide, >95% ee (15 minutes) and Examples 88b (5S,7R)-5-(4-ethylphenyl)-N-(4-fluoro-3-methylbenzyl)-7-(trifluoromethyl)-4,5,6,7-tetrahydropyrazolo[1,5-a]pyrimidine-3-carboxamide, >95% ee (22 minutes). [Column: Chiralpack IC, 250×20 mm, temperature 25° C., mobile phase: hexane/ethanol 95/5, Flow rate: 18 ml/minute, detection wavelength: 254 nm, and injection of 70 mg.

### Examples 89a and 89b

### Enantiomers a and b of cis-5-(4-ethylphenyl)-N-(4-methoxybenzyl)-7-methyl-4,5,6,7-tetrahydropyrazolo[1,5-a]pyrimidine-3-carboxamide

Cis-5-(4-ethylphenyl)-N-(4-methoxybenzyl)-7-methyl-4,5,6,7-tetrahydropyrazolo[1,5-a]pyrimidine-3-carboxamide was subjected to semi-preparative high performance liquid chromatography (HPLC) to afford the optically pure enantiomers Example 89a (5R,7R)-5-(4-ethylphenyl)-N-(4-methoxybenzyl)-7-methyl-4,5,6,7-tetrahydropyrazolo[1,5-a]pyrimidine-3-carboxamide, >95% ee (14 minutes) and Examples 89b (5S,7S)-5-(4-ethylphenyl)-N-(4-methoxybenzyl)-7-methyl-4,5,6,7-tetrahydropyrazolo[1,5-a]pyrimidine-3-carboxamide, >95% ee (21 minutes). [Column: Chiralpack IC, 250×20 mm, temperature 25° C., mobile phase: hexane/ethanol 80/20, Flow rate: 18 ml/minute, detection wavelength: 254 nm, and injection of 35 mg.

### Examples 90a and 90b

### Enantiomers a and b of cis-5-(4-ethylphenyl)-N-((5-methylpyridin-2-yl)methyl)-7-(trifluoromethyl)-4,5,6,7-tetrahydropyrazolo[1,5-a]pyrimidine-3-carboxamide

Cis-5-(4-ethylphenyl)-N-((5-methylpyridin-2-yl)methyl)-7-(trifluoromethyl)-4,5,6,7-tetrahydropyrazolo[1,5-a]pyrimidine-3-carboxamide was subjected to semi-preparative high performance liquid chromatography (HPLC) to afford the optically pure enantiomers Example 90a (5R,7S)-5-(4-ethylphenyl)-N-((5-methylpyridin-2-yl)methyl)-7-(trifluoromethyl)-4,5,6,7-tetrahydropyrazolo[1,5-a]pyrimidine-3-carboxamide, >95% ee (9 minutes) and Example 90b (5R,7S)-5-(4-ethylphenyl)-N-((5-methylpyridin-2-yl)methyl)-7-(trifluoromethyl)-4,5,6,7-tetrahydropyrazolo[1,5-a]pyrimidine-3-carboxamide, >95% ee (15 minutes). [Column: Chiralpack IC, 250×20 mm, temperature 25° C., mobile phase: hexane/ethanol 95/5, Flow rate: 18 ml/minute, detection wavelength: 254 nm, and injection of 100 mg.

### Examples 91a and 91b

### Enantiomers a and b of cis-7-(tert-butyl)-5-(4-ethylphenyl)-N-(4-methoxybenzyl)-4,5,6,7-tetrahydropyrazolo[1,5-a]pyrimidine-3-carboxamide

Cis-7-(tert-butyl)-5-(4-ethylphenyl)-N-(4-methoxybenzyl)-4,5,6,7-tetrahydropyrazolo[1,5-a]pyrimidine-3-carboxamide was subjected to semi-preparative high performance liquid chromatography (HPLC) to afford the optically pure enantiomers Example 91a (5R,7S)-7-(tert-butyl)-5-(4-ethylphenyl)-N-(4-methoxybenzyl)-4,5,6,7-tetrahydropyrazolo[1,5-a]pyrimidine-3-carboxamide, >95% ee (17 minutes) and Example 91b (5S,7R)-7-(tert-butyl)-5-(4-ethylphenyl)-N-(4-methoxybenzyl)-4,5,6,7-tetrahydropyrazolo[1,5-a]pyrimidine-3-carboxamide >95% ee (22 minutes). [Column: Chiralpack IC, 250×20 mm, temperature 25° C., mobile phase: hexane/ethanol 93/7, Flow rate: 18 ml/minute, detection wavelength: 254 nm, and injection of 60 mg.

### Examples 92a and 92b

### Enantiomers a and b of cis-5-(4-ethylphenyl)-7-(trifluoromethyl)-N-(4-(trifluoromethyl)benzyl)-4,5,6,7-tetrahydropyrazolo[1,5-a]pyrimidine-3-carboxamide

Cis-5-(4-ethylphenyl)-7-(trifluoromethyl)-N-(4-(trifluoromethyl)benzyl)-4,5,6,7-tetrahydropyrazolo[1,5-a]pyrimidine-3-carboxamide was subjected to semi-preparative high performance liquid chromatography (HPLC) to afford the optically pure enantiomers Example 92a (5R,7S)-5-(4-ethylphenyl)-7-(trifluoromethyl)-N-(4-(trifluoromethyl)benzyl)-4,5,6,7-tetrahydropyrazolo[1,5-a]pyrimidine-3-carboxamide, >95% ee (9 minutes) and Example 92b (5S,7R)-5-(4-ethylphenyl)-7-(trifluoromethyl)-N-(4-(trifluoromethyl)benzyl)-4,5,6,7-tetrahydropyrazolo[1,5-a]pyrimidine-3-carboxamide, >95% ee (15 minutes). [Column: Chiralpack IC, 250×20 mm, temperature 25° C., mobile phase: hexane/ethanol 95/5, Flow rate: 18 ml/minute, detection wavelength: 254 nm, and injection of 55 mg.

### Examples 93a and 93b

### Enantiomers a and b of cis-5-(4-ethylphenyl)-N-((6-methylpyridin-3-yl)methyl)-7-(trifluoromethyl)-4,5,6,7-tetrahydropyrazolo[1,5-a]pyrimidine-3-carboxamide

Cis-5-(4-ethylphenyl)-N-((6-methylpyridin-3-yl)methyl)-7-(trifluoromethyl)-4,5,6,7-tetrahydropyrazolo[1,5-a]pyrimidine-3-carboxamide was subjected to semi-preparative high performance liquid chromatography (HPLC) to afford the optically pure enantiomers Example 93a (5R,7S)-5-(4-ethylphenyl)-N-((6-methylpyridin-3-yl)methyl)-7-(trifluoromethyl)-4,5,6,7-tetrahydropyrazolo[1,5-a]pyrimidine-3-carboxamide, >95% ee (10 minutes) and Example 93b (5S,7R)-5-(4-ethylphenyl)-N-((6-methylpyridin-3-yl)methyl)-7-(trifluoromethyl)-4,5,6,7-tetrahydropyrazolo[1,5-a]pyrimidine-3-carboxamide, >95% ee (15 minutes). [Column: Chiralpack IC, 250×20 mm, temperature 25° C., mobile phase: hexane/ethanol 80/20, Flow rate: 18 ml/minute, detection wavelength: 254 nm, and injection of 55 mg.

### Examples 94a and 94b

### Enantiomers a and b of cis-5-(4-ethylphenyl)-N-((5-fluoropyridin-2-yl)methyl)-7-(trifluoromethyl)-4,5,6,7-tetrahydropyrazolo[1,5-a]pyrimidine-3-carboxamide

Cis-5-(4-ethylphenyl)-N-((5-fluoropyridin-2-yl)methyl)-7-(trifluoromethyl)-4,5,6,7-tetrahydropyrazolo[1,5-a]pyrimidine-3-carboxamide was subjected to semi-preparative high performance liquid chromatography (HPLC) to afford the optically pure enantiomers Example 94a (5R,7S)-5-(4-ethylphenyl)-N-((5-fluoropyridin-2-yl)methyl)-7-(trifluoromethyl)-4,5,6,7-tetrahydropyrazolo[1,5-a]pyrimidine-3-carboxamide, >95% ee (16 minutes) and Example 94b (5S,7R)-5-(4-ethylphenyl)-N-((5-fluoropyridin-2-yl)methyl)-7-(trifluoromethyl)-4,5,6,7-tetrahydropyrazolo[1,5-a]pyrimidine-3-carboxamide >95% ee (23 minutes). [Column: Chiralpack IC, 250×20 mm, temperature 25° C., mobile phase: hexane/ethanol 90/10, Flow rate: 18 ml/minute, detection wavelength: 254 nm, and injection of 100 mg.

### Example 95

### (5R,7S)—N-(benzo[d][1,3]dioxol-5-ylmethyl)-5-(4-ethylphenyl)-7-(trifluoromethyl)-4,5,6,7-tetrahydropyrazolo[1,5-a]pyrimidine-3-carboxamide

To a solution of Intermediate 21 (4.5 g, 13.26 mmol) in N,N-Dimethylformamide (DMF) (25 mL) was added HATU (CARBOSYNTH, 6.05 g, 15.91 mmol) and N,N-diisopropylethylamine (FLUKA, 6.95 mL, 39.8 mmol), the mixture was stirred at room temperature for 30 min then piperonylamine (ALDRICH, 2.481 mL, 19.89 mmol) was added and the mixture was stirred at 50° C. overnight. After cooling to room temperature, reaction mixture was diluted with tert-butyl methyl ether (50 mL) and washed with sat. NH₄Cl (25 mL) and sat. NaCl (25 mL). When it was washed with NaCl a solid appeared and it was necessary to dilute with more tert-butyl methyl ether (100 mL). The organic layers were dried over MgSO₄, filtered and evaporated. The crude was purified by grinding with a mixture of tert-butyl methyl ether (15 mL) and hexane (30 mL), but solid filtrate was pale brown and it was repurified solving solid with Dichloromethane (5 mL) and precipitating with Hexane (30 mL). Solid was filtered and dried in a vacuum drying overnight at 40° C. to obtain a white solid. (4.1 g, 862 mmol, 65.4%).

¹H NMR (400 MHz, CDCl₃) δ ppm: 7.51 (s, 1H), 7.35 (d, 2H), 7.23 (d, 2H), 6.82 (s, 1H), 6.77 (s, 2H), 6.67 (bs, 1H), 5.94 (s, 2H), 5.82-5.79 (m, 1H), 4.88-4.80 (m, 1H), 4.55-4.52 (m, 1H), 4.45-4.43 (m, 2H), 2.70-2.64 (m, 2H), 2.55-2.50 (m, 1H), 2.42-2.33 (m, 1H), 1.25 (t, 3H). [ES+MS] m/z 473 (M+H)⁺. [α]_{D}²⁵=+43.7 (c=0.6, EtOH).

### Example 96

### (5R,7S)—N-(benzo[d][1,3]dioxol-5-ylmethyl)-5-(4-ethylphenyl)-7-(trifluoromethyl)-4,5,6,7-tetrahydropyrazolo[1,5-a]pyrimidine-3-carboxamide

The title compound was prepared by a method analogous to that described for Example 1. Intermediate 21 (150 mg, 0.442 mmol), HATU (CARBOSYNTH, 202 mg, 0.530 mmol), N,N-diisopropylethylamine (FLUKA, 0.232 mL, 1.326 mmol) and Piperonylamine (Aldrich, 0.083 mL, 0.663 mmol) as starting reactants. The title compound (140 mg, 0.296 mmol, 67%) was obtained as a white solid. ¹H NMR (400 MHz, CDCl₃) δ ppm: 7.51 (s, 1H), 7.35 (d, 2H), 7.23 (d, 2H), 6.82 (s, 1H), 6.77 (d, 2H), 6.67 (bs, 1H), 5.94 (s, 2H), 5.80 (ts, 1H), 4.88-4.80 (m, 1H), 4.55-4.52 (m, 1H), 4.49-4.40 (m, 2H), 2.67 (q, 2H), 2.55-2.50 (m, 1H), 2.42-2.33 (m, 1H), 1.25 (t, 3H). [ES+MS] m/z 473 (M+H)⁺.

### Example 97

### (5R,7S)-5-(4-ethylphenyl)-N-(2-fluoro-4-methylbenzyl)-7-(trifluoromethyl)-4,5,6,7-tetrahydropyrazolo[1,5-a]pyrimidine-3-carboxamide

The title compound was prepared by a method analogous to that described for Example 1. Intermediate 21 (90 mg, 0.265 mmol), HATU (CARBOSYNTH, 121 mg, 0.318 mmol), N,N-diisopropylethylamine (FLUKA, 0.139 mL, 0.796 mmol) and (2-fluoro-4-methylphenyl)methanamine (Fluorochemicals Ltd. 55.4 mg, 0.398 mmol) as starting reactants. The title compound (87 mg, 0.189 mmol, 71%) was obtained as a white solid.

¹H NMR (400 MHz, CDCl₃) δ ppm: 7.52 (s, 1H), 7.34 (d, 2H), 7.26 (d, 1H), 7.22 (d, 2H), 6.92-6.86 (m, 2H), 6.64 (bs, 1H), 5.86 (t, 1H), 4.86-4.80 (m, 1H), 4.55-4.49 (m, 3H), 2.67 (q, 2H), 2.55-2.49 (m, 1H), 2.41-2.33 (m, 1H), 2.33 (s, 3H), 1.25 (t, 3H). [ES+MS] m/z 461 (M+H)⁺.

### Example 98

### (5R,7S)—N-(3,4-difluorobenzyl)-5-(4-ethylphenyl)-7-(trifluoromethyl)-4,5,6,7-tetrahydropyrazolo[1,5-a]pyrimidine-3-carboxamide

The title compound was prepared by a method analogous to that described for Example 1. Intermediate 21 (50 mg, 0.147 mmol), HATU (CARBOSYNTH, 67.2 mg, 0.177 mmol), N,N-diisopropylethylamine (FLUKA, 0.077 mL, 0.442 mmol) and 2,3-difluorobenzylamine (ALDRICH, 31.6 mg, 0.22 mmol) as starting reactants. The title compound (55.0 mg, 0.118 mmol, 80%) was obtained as a white solid. ¹H NMR (400 MHz, CDCl₃) δ ppm: 7.54 (s, 1H), 7.35 (d, 2H), 7.23 (d, 2H), 7.08-7.17 (m, 2H), 7.05-7.02 (m, 1H), 6.63 (bs, 1H), 5.92-5.89 (m, 1H), 4.87-4.82 (m, 1H), 4.56-4.48 (m, 3H), 2.67 (q, 2H), 2.57-2.50 (m, 1H), 2.43-2.33 (m, 1H), 1.25 (t, 3H). [ES+MS] m/z 465 (M+H)⁺.

### Example 99

### (5R,7S)-5-(4-ethylphenyl)-N-(4-methylbenzyl)-7-(trifluoromethyl)-4,5,6,7-tetrahydropyrazolo[1,5-a]pyrimidine-3-carboxamide

The title compound was prepared by a method analogous to that described for Example 1. Intermediate 21 (250 mg, 0.737 mmol), HATU (CARBOSYNTH, 336 mg, 0.884 mmol), N,N-diisopropylethylamine (FLUKA, 0.386 mL, 2.211 mmol) and 4-Methylbenzylamine (ALDRICH, 116 mg, 0.958 mmol) as starting reactants. The title compound (275.0 mg, 0.622 mmol, 84%) was obtained as a white solid. ¹H NMR (400 MHz, CDCl₃) δ ppm: 7.51 (s, 1H), 7.35 (d, 2H), 7.24-7.21 (m, 4H), 7.15 (d, 2H), 6.68 (bs, 1H), 5.81-5.79 (m, 1H), 4.87-4.81 (m, 1H), 4.55-4.49 (m, 3H), 2.67 (q, 2H), 2.57-2.50 (m, 1H), 2.43-2.34 (m, 1H), 2.34 (s, 3H), 1.25 (t, 3H). [ES+MS] m/z 443 (M+H)⁺.

### Example 100

### (5R,7S)-5-(4-ethylphenyl)-N-((5-methylfuran-2-yl)methyl)-7-(trifluoromethyl)-4,5,6,7-tetrahydropyrazolo[1,5-a]pyrimidine-3-carboxamide

The title compound was prepared by a method analogous to that described for Example 1. Intermediate 21 (75 mg, 0.221 mmol), HATU (CARBOSYNTH, 101 mg, 0.265 mmol), N,N-diisopropylethylamine (FLUKA, 0.116 mL, 0.663 mmol) and 5-Methylfurfurylamine (Aldrich, 0.037 mL, 0.332 mmol) as starting reactants. The title compound (65 mg, 0.150 mmol, 68%) was obtained as a off-white solid. ¹H NMR (400 MHz, CDCl₃) δ ppm: 7.53 (s, 1H), 7.35 (d, 2H), 7.23 (d, 2H), 6.65 (bs, 1H), 6.12 (d, 1H), 5.90 (d, 1H), 5.79 (ts, 1H), 4.88-4.79 (m, 1H), 4.54-4.51 (m, 1H), 4.48-4.42 (m, 2H), 2.67 (q, 2H), 2.54-2.50 (m, 1H), 2.41-2.32 (m, 1H), 2.28 (s, 3H), 1.25 (t, 3H). [ES+MS] m/z 433 (M+H)⁺.

### Example 101

### (5R,7S)—N-((3,5-difluoropyridin-2-yl)methyl)-5-(4-ethylphenyl)-7-(trifluoromethyl)-4,5,6,7-tetrahydropyrazolo[1,5-a]pyrimidine-3-carboxamide

The title compound was prepared by a method analogous to that described for Example 1 Intermediate 21 (140 mg, 0.413 mmol), HATU (CARBOSYNTH, 235 mg, 0.619 mmol), N,N-diisopropylethylamine (FLUKA, 0.432 mL, 2.474 mmol) and 3,5-difluoropyridin-2-yl)methanamine hydrochloride (prepared as described in WO2005/66126; 115 mg, 0.637 mmol) as starting reactants. The title compound (138.9 mg, 0.298 mmol, 72%) was obtained as a off-white solid. ¹H NMR (400 MHz, CDCl₃) δ ppm: 8.32 (d, 1H), 7.65 (s, 1H), 7.35 (d, 2H), 7.26-7.16 (m, 3H), 6.70 (bt, 1H), 6.63 (bs, 1H), 4.90-4.79 (m, 1H), 4.70 (d, 2H), 4.53 (dd, 1H), 2.72-2.63 (m, 2H), 2.57-2.48 (m, 1H), 2.43-2.31 (m, 1H), 1.25 (t, 3H). [ES+MS] m/z 466 (M+H)⁺.

### Example 102

### (5R,7S)-5-(4-ethylphenyl)-N-((6-methoxypyridin-3-yl)methyl)-7-(trifluoromethyl)-4,5,6,7-tetrahydropyrazolo[1,5-a]pyrimidine-3-carboxamide

The title compound was prepared by a method analogous to that described for Example 1. Intermediate 21 (50 mg, 0.147 mmol), HATU (CARBOSYNTH, 67 mg, 0.177 mmol), N,N-diisopropylethylamine (FLUKA, 0.077 mL, 0.442 mmol) and (6-methoxypyridin-3-yl)methanamine (JW PHARMLAB, 0.025 mL, 0.221 mmol) as starting reactants. The title compound (60 mg, 0.131 mmol, 89%) was obtained as a white solid. ¹H NMR (400 MHz, CDCl₃) δ ppm: 8.11 (s, 1H), 7.59-7.56 (m, 1H), 7.51 (s, 1H), 7.35 (d, 2H), 7.23 (d, 2H), 6.72 (d, 1H), 6.64 (bs, 1H), 5.82 (ts, 1H), 4.87-4.80 (m, 1H), 4.55-4.53 (m, 1H), 4.50-4.42 (m, 2H), 3.92 (s, 3H), 2.67 (q, 2H), 2.55-2.50 (m, 1H), 2.42-2.32 (m, 1H), 1.25 (t, 3H). [ES+MS] m/z 460 (M+H)⁺.

### Example 103

### (5R,7S)—N-((6-chloropyridin-3-yl)methyl)-5-(4-ethylphenyl)-7-(trifluoromethyl)-4,5,6,7-tetrahydropyrazolo[1,5-a]pyrimidine-3-carboxamide

The title compound was prepared by a method analogous to that described for Example 1. Intermediate 21 (60 mg, 0.177 mmol), HATU (CARBOSYNTH, 81 mg, 0.212 mmol), N,N-diisopropylethylamine (FLUKA, 0.093 mL, 0.530 mmol) and (6-chloropyridin-3-yl)methanamine (Aldrich, 38 mg, 0.265 mmol) as starting reactants. The title compound (53 mg, 0.114 mmol, 65%) was obtained as a off-white solid. ¹H NMR (400 MHz, CDCl₃) δ ppm: 8.34 (s, 1H), 7.67-7.64 (m, 1H), 7.55 (s, 1H), 7.35 (d, 2H), 7.29 (d, 1H), 7.23 (d, 2H), 6.60 (bs, 1H), 5.97 (ts, 1H), 4.89-4.80 (m, 1H), 4.58-4.48 (m, 3H), 2.67 (q, 2H), 2.56-2.51 (m, 1H), 2.42-2.33 (m, 1H), 1.25 (t, 3H). [ES+MS] m/z 464 (M+H)⁺.

### Example 104

### (5R,7S)-5-(4-ethylphenyl)-N-((5-fluoro-6-methoxypyridin-3-yl)methyl)-7-(trifluoromethyl)-4,5,6,7-tetrahydropyrazolo[1,5-a]pyrimidine-3-carboxamide

The title compound was prepared by a method analogous to that described for Example 1. Intermediate 21 (150 mg, 0.442 mmol), HATU (CARBOSYNTH, (202 mg, 0.530 mmol), N,N-diisopropylethylamine (FLUKA, 0.232 mL, 1.326 mmol) and (5-fluoro-6-methoxypyridin-3-yl)methanamine (ASYCHEM; 87 mg, 0.530 mmol) as starting reactants. The title compound (87 mg, 0.194 mmol, 40%) was obtained as a solid.

¹H NMR (400 MHz, CDCl₃) δ ppm: 7.88 (d, 1H), 7.53 (s, 1H), 7.40-7.33 (m, 3H), 7.24 (d, 2H), 6.63 (s, 1H), 5.88 (t, 1H), 4.90-4.80 (m, 1H), 4.58-4.52 (m, 1H), 4.51-4.40 (m, 2H), 4.01 (s, 3H), 2.72-2.64 (m, 2H), 2.58-2.50 (m, 1H), 2.44-2.33 (m, 1H), 1.26 (t, 3H). [ES+MS] m/z 478 (M+H)⁺.

### Example 105

### (5R,7S)-5-(4-ethylphenyl)-N-((5-fluoro-6-methylpyridin-2-yl)methyl)-7-(trifluoromethyl)-4,5,6,7-tetrahydropyrazolo[1,5-a]pyrimidine-3-carboxamide

The title compound was prepared by a method analogous to that described for Example 1. Method B. Intermediate 21 (150 mg, 0.442 mmol), HATU (CARBOSYNTH, 202 mg, 0.530 mmol), N,N-diisopropylethylamine (FLUKA, 0.232 mL, 1.326 mmol) and (5-fluoro-6-methylpyridin-2-yl)methanamine (prepared as described in WO2005/66126;

238 mg, 1.698 mmol) as starting reactants. The title compound (60 mg, 0.130 mmol, 29%) was obtained as a beige solid. ¹H NMR (400 MHz, CDCl₃) δ ppm: 7.63 (s, 1H), 7.34 (d, 2H), 7.31-7.27 (m, 1H), 7.22 (d, 2H), 7.15-7.12 (m, 1H), 6.67-6.64 (m, 1H), 6.61 (bs, 1H), 4.89-4.80 (m, 1H), 4.62-4.51 (m, 3H), 2.67 (q, 2H), 2.55-2.50 (m, 4H), 2.42-2.32 (m, 1H), 1.25 (t, 3H). [ES+MS] m/z 462 (M+H)⁺.

### Example 106

### (5R,7S)-5-(4-ethylphenyl)-N-((3-fluoro-5-methylpyridin-2-yl)methyl)-7-(trifluoromethyl)-4,5,6,7-tetrahydropyrazolo[1,5-a]pyrimidine-3-carboxamide

The title compound was prepared by a method analogous to that described for Example 1. Method B, Intermediate 21 (200 mg, 0.589 mmol), HATU (CARBOSYNTH, 269 mg, 0.707 mmol), N,N-diisopropylethylamine (FLUKA, 0.309 mL, 1.768 mmol) and (3-fluoro-5-methylpyridin-2-yl)methanamine (Ellanova, 257 mg, 1.834 mmol) as starting reactants. The title compound (170 mg, 0.368 mmol, 60%) was obtained as a pale yellow solid.

¹H NMR (400 MHz, CDCl₃) δ ppm: 8.24 (s, 1H), 7.73 (s, 1H), 7.36-7.32 (m, 3H), 7.22 (d, 2H), 7.12 (bs, 1H), 6.61 (s, 1H), 4.90-4.80 (m, 1H), 4.72 (d, 2H), 4.55-4.48 (m, 1H), 2.67 (q, 2H), 2.56-2.47 (m, 1H), 2.42-2.30 (m, 4H), 1.25 (t, 3H).

[ES+MS] m/z 462 (M+H)⁺.

### Example 107

The following compounds were also prepared:
Cis-5-(4-methoxyphenyl)-N-(4-methylbenzyl)-7-(trifluoromethyl)-4,5,6,7-tetrahydropyrazolo[1,5-a]pyrimidine-3-carboxamide
Cis-5-(4-ethylphenyl)-7-(trifluoromethyl)-N-((6-(trifluoromethyl)pyridin-3-yl)methyl)-4,5,6,7-tetrahydropyrazolo[1,5-a]pyrimidine-3-carboxamide;
Cis-N-((2,5-dimethylfuran-3-yl)methyl)-5-(4-ethylphenyl)-7-(trifluoromethyl)-4,5,6,7-tetrahydropyrazolo[1,5-a]pyrimidine-3-carboxamide;
Cis-7-(difluoromethyl)-5-(4-ethylphenyl)-N-(4-methylbenzyl)-4,5,6,7-tetrahydropyrazolo[1,5-a]pyrimidine-3-carboxamide;
Cis-5-(4-ethylphenyl)-N-((3-fluoropyridin-4-yl)methyl)-7-(trifluoromethyl)-4,5,6,7-tetrahydropyrazolo[1,5-a]pyrimidine-3-carboxamide;
N-(3,4-difluorobenzyl)-5-(4-ethylphenyl)-7-(trifluoromethyl)-4,5,6,7-tetrahydropyrazolo[1,5-a]pyrimidine-3-carboxamide;
5-(4-ethylphenyl)-N-(4-methylbenzyl)-7-(trifluoromethyl)-4,5,6,7-tetrahydropyrazolo[1,5-a]pyrimidine-3-carboxamide;
N-(cyclohexylmethyl)-5-(4-ethylphenyl)-7-(trifluoromethyl)-4,5,6,7-tetrahydropyrazolo[1,5-a]pyrimidine-3-carboxamide;
5-(4-ethylphenyl)-N-((5-methylpyridin-2-yl)methyl)-7-(trifluoromethyl)-4,5,6,7-tetrahydropyrazolo[1,5-a]pyrimidine-3-carboxamide;
Cis-7-(difluoromethyl)-5-(4-ethylphenyl)-N-(4-methoxybenzyl)-4,5,6,7-tetrahydropyrazolo[1,5-a]pyrimidine-3-carboxamide;
Cis-N-(benzo[d][1,3]dioxol-5-ylmethyl)-7-(difluoromethyl)-5-(4-ethylphenyl)-4,5,6,7-tetrahydropyrazolo[1,5-a]pyrimidine-3-carboxamide;
Cis-5-(4-ethylphenyl)-N-((6-methylpyridin-2-yl)methyl)-7-(trifluoromethyl)-4,5,6,7-tetrahydropyrazolo[1,5-a]pyrimidine-3-carboxamide;
Cis-5-(4-ethylphenyl)-N-((4-methylpyridin-2-yl)methyl)-7-(trifluoromethyl)-4,5,6,7-tetrahydropyrazolo[1,5-a]pyrimidine-3-carboxamide;
N-(benzo[d][1,3]dioxol-5-ylmethyl)-5-(4-bromophenyl)-7-(trifluoromethyl)-4,5,6,7-tetrahydropyrazolo[1,5-a]pyrimidine-3-carboxamide;
Cis-5-(4-ethylphenyl)-N-((3-fluoropyridin-2-yl)methyl)-7-(trifluoromethyl)-4,5,6,7-tetrahydropyrazolo[1,5-a]pyrimidine-3-carboxamide;
(5S,7R)-5-(4-ethylphenyl)-N-((5-methylthiophen-2-yl)methyl)-7-(trifluoromethyl)-4,5,6,7-tetrahydropyrazolo[1,5-a]pyrimidine-3-carboxamide;
(5S,7R)-7-(difluoromethyl)-5-(4-ethylphenyl)-N-(4-methoxybenzyl)-4,5,6,7-tetrahydropyrazolo[1,5-a]pyrimidine-3-carboxamide;
(5S,7R)-5-(4-ethylphenyl)-N-(4-fluorobenzyl)-7-(trifluoromethyl)-4,5,6,7-tetrahydropyrazolo[1,5-a]pyrimidine-3-carboxamide;
(5S,7R)—N-(3,4-difluorobenzyl)-5-(p-tolyl)-7-(trifluoromethyl)-4,5,6,7-tetrahydropyrazolo[1,5-a]pyrimidine-3-carboxamide;
(5S,7R)-5-(4-ethylphenyl)-N-((5-methylfuran-2-yl)methyl)-7-(trifluoromethyl)-4,5,6,7-tetrahydropyrazolo[1,5-a]pyrimidine-3-carboxamide;
(5S,7R)-5-(4-ethylphenyl)-N-(3-methylbenzyl)-7-(trifluoromethyl)-4,5,6,7-tetrahydropyrazolo[1,5-a]pyrimidine-3-carboxamide;
(5S,7R)-5-(4-ethylphenyl)-N-(3-fluoro-4-methylbenzyl)-7-(trifluoromethyl)-4,5,6,7-tetrahydropyrazolo[1,5-a]pyrimidine-3-carboxamide;
Cis-N-((4,6-dimethylpyridin-3-yl)methyl)-5-(4-ethylphenyl)-7-(trifluoromethyl)-4,5,6,7-tetrahydropyrazolo[1,5-a]pyrimidine-3-carboxamide;
Cis-5-(4-ethylphenyl)-N-((2-methylfuran-3-yl)methyl)-7-(trifluoromethyl)-4,5,6,7-tetrahydropyrazolo[1,5-a]pyrimidine-3-carboxamide;
(5S,7R)-7-(tert-butyl)-5-(4-ethylphenyl)-N-(4-methoxybenzyl)-4,5,6,7-tetrahydropyrazolo[1,5-a]pyrimidine-3-carboxamide;
(5S,7S)-5-(4-ethylphenyl)-N-(4-methoxybenzyl)-7-methyl-4,5,6,7-tetrahydropyrazolo[1,5-a]pyrimidine-3-carboxamide;
(5S,7R)-5-(4-ethylphenyl)-N-(4-fluoro-3-methylbenzyl)-7-(trifluoromethyl)-4,5,6,7-tetrahydropyrazolo[1,5-a]pyrimidine-3-carboxamide;
(5S,7R)-5-(4-ethylphenyl)-N-(3-fluorobenzyl)-7-(trifluoromethyl)-4,5,6,7-tetrahydropyrazolo[1,5-a]pyrimidine-3-carboxamide;
(5S,7R)-5-(4-ethylphenyl)-N-((5-fluoropyridin-2-yl)methyl)-7-(trifluoromethyl)-4,5,6,7-tetrahydropyrazolo[1,5-a]pyrimidine-3-carboxamide;
(5S,7R)—N-(benzo[d][1,3]dioxol-5-ylmethyl)-7-(difluoromethyl)-5-(4-ethylphenyl)-4,5,6,7-tetrahydropyrazolo[1,5-a]pyrimidine-3-carboxamide;
(5S,7R)-5-(4-ethylphenyl)-7-(trifluoromethyl)-N-(4-(trifluoromethyl)benzyl)-4,5,6,7-tetrahydropyrazolo[1,5-a]pyrimidine-3-carboxamide; and
(5S,7R)-5-(4-ethylphenyl)-N-((6-methylpyridin-3-yl)methyl)-7-(trifluoromethyl)-4,5,6,7-tetrahydropyrazolo[1,5-a]pyrimidine-3-carboxamide.

### Biological Activity

Mycobacterium tuberculosis H37Rv Inhibition Assay (Whole Cell Assay)

The measurement of the minimum inhibitory concentration (MIC) for each tested compound was performed in 96 wells flat-bottom, polystyrene microtiter plates. Ten two-fold drug dilutions in neat DMSO starting at 5 μM were performed. Five μl of these drug solutions were added to 95 μl of Middlebrook 7H9 medium. (Lines A-H, rows 1-10 of the plate layout). Isoniazid was used as a positive control, 8 two-fold dilution of Isoniazid starting at 160 μg/ml was prepared and 5 μl of this control curve was added to 95 μl of Middlebrook 7H9 medium (Difco catalogue ref. 271310). (Row 11, lines A-H). 5 μl of neat DMSO were added to row 12 (growth and Blank controls).

The inoculum was standardised to approximately 1×107 cfu/ml and diluted 1 in 100 in Middlebrook 7H9 broth (Middlebrook ADC enrichment, a dehydrated culture media which supports growth of mycobacterial species available from Becton Dickinson Catalogue Ref. 211887), to produce the final inoculum of H37Rv strain (ATCC25618). One hundred μl of this inoculum was added to the entire plate but G-12 and H-12 wells (Blank controls). All plates were placed in a sealed box to prevent drying out of the peripheral wells and they were incubated at 37° C. without shaking for six days. A resazurin solution was prepared by dissolving one tablet of resazurin (Resazurin Tablets for Milk Testing; Ref 330884Y VWR International Ltd) in 30 ml sterile PBS (phosphate buffered saline). 25 μl of this solution was added to each well. Fluorescence was measured (Spectramax M5 Molecular Devices, Excitation 530 nm, Emission 590 nm) after 48 hours to determine the MIC value.

Results of the Mycobacterium tuberculosis H37Rv Inhibition Assay (Whole Cell Assay)

Unless otherwise stated hereinbelow, all Examples were tested in the whole cell assay.

Examples 1, 1a, 2, 3, 4, 5, 6, 7, 7a, 8, 9, 12, 15, 16, 17, 21, 22, 23, 24, 25, 30, 31, 32, 33, 34, 35, 37, 38, 39, 40, 41, 58, 59, 60-70, 71, 72, 78, 79, 80-90, 91-100, and 101-105 described hereinabove were found to have an MIC value of 1 μM or less. For example, Example 2 was found to have an MIC value of 0.1 μM.

Examples 11, 10, 13, 14, 18, 19, 20, 26, 27, 28, 29, 36, 73-77 and 106 described hereinabove were found to have an MIC value of between 1 μM and 2 μM.

Examples 1b, 7b, 42-55, comparative Examples 56′ and 57′, and the compounds of Example 106 described hereinabove were found to have an MIC value of greater than 2 μM. Examples 1b, 7b, 42-55 and the compounds of Ex 107 may have utility in the preparation of other compounds of Formula (I).

In one aspect, compounds of the invention have an MIC value of 2 μM or less in the Mycobacterium tuberculosis H37Rv Inhibition Assay (Whole Cell Assay).

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

1.-15. (canceled)

16. A method of treatment of tuberculosis in a mammal, which method comprises the administration to a mammal in need of such treatment of an effective amount of a compound of Formula (I), or a pharmaceutically acceptable salt thereof: 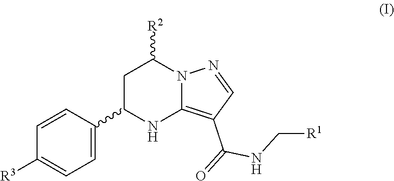
wherein:
R¹ represents a group selected from:
i) phenyl optionally substituted with one or two substituents independently selected from Me, OMe, CF₃, F, Cl and NMe₂;
ii) (uranyl, thiophenyl, pyrrolyl, pyridyl, cyclohexyl or naphthyl, each of which is optionally substituted with one or two substituents independently selected from Me, OMe, CF₃, F, Cl and NMe₂;
iii) benzo[1,3]-dioxo5-yl: 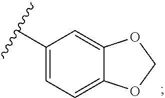 [SMILES: *c1ccc2c(c1)OCO2]
and
iv) 2,3-dihydrobenzo[1,4]dioxin-6-yl: 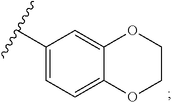 [SMILES: *c1ccc2c(c1)OCCO2]
R² represents CF₃, C₁₋₄alkyl, or CHF₂;
with the proviso that when R¹ represents optionally substituted furanyl, thiophenyl, pyrrolyl, pyridyl or naphthyl, then R³ represents Et; and
when R¹ represents optionally substituted cyclohexyl, then R³ represents Et or Me;
otherwise R³ represents Et, Me, Br or OMe.

17. The method of claim 16 wherein said compound is Formula (IA) or a pharmaceutically acceptable salt thereof: 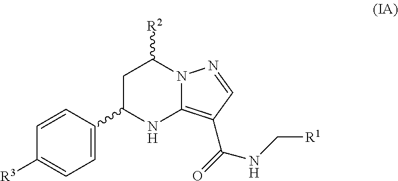
wherein:
R¹ represents a group selected from:
i) phenyl optionally substituted with one or two substituents independently selected from Me, OMe, CF₃, F and NMe₂, or phenyl substituted with either a) one Cl substituent at the 4-position, or b) two Cl substituents at the 3- and 4-positions;
ii) furanyl, thiophenyl, pyrrolyl, pyridyl or naphthyl, each of which is optionally substituted with one or two substituents independently selected from Me, OMe, CF₃, F, Cl and NMe₂; or cyclohexyl substituted with one or two substituents independently selected from Me, OMe, CF₃, F, Cl and NMe₂;
iii) benzo[1,3]dioxo5-yl: 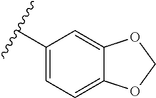 [SMILES: *c1ccc2c(c1)OCO2]
and
iv) 2,3-dihydrobenzo[1,4]dioxin-6-yl: 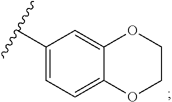 [SMILES: *c1ccc2c(c1)OCCO2]
R² represents CF₃, C₁₋₄alkyl, or CHF₂;
with the proviso that when R¹ represents optionally substituted furanyl, thiophenyl, pyrrolyl, pyridyl or naphthyl, then R³ represents Et; and
when R¹ represents substituted cyclohexyl, then R³ represents Et or Me;
otherwise R³ represents Et, Me, Br or OMe.

18. The method of claim 16 wherein the compound is Formula (IB) or a pharmaceutically acceptable salt thereof: 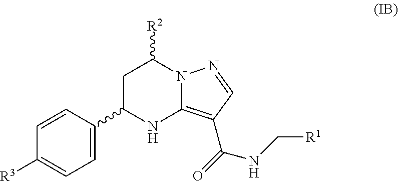
wherein:
R¹ represents a group selected from:
i) phenyl optionally substituted with one or two substituents independently selected from Me, CF₃, F and NMe₂ or phenyl substituted with either a) one Cl substituent at the 4-position, or b) two Cl substituents at the 3- and 4-positions
ii) (uranyl, pyrrolyl, pyridyl or naphthyl, each of which is optionally substituted with one or two substituents independently selected from Me, OMe, CF₃, F, Cl and NMe₂: or cyclohexyl or thiophenyl, each of which is substituted with one or two substituents independently selected from Me, OMe, CF₃, F, Cl and NMe₂; and
iii) 2,3-dihydrobenzo[1,4]dioxin-6-yl: 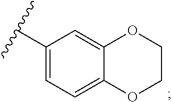 [SMILES: *c1ccc2c(c1)OCCO2]
R² represents CF₃, C₁₋₄alkyl, or CHF₂;
with the proviso that when R¹ represents optionally substituted furanyl, pyrrolyl, pyridyl or naphthyl, or substituted thiophenyl, or when R² represents CHF₂, then R³ represents Et; and
when R¹ represents substituted cyclohexyl, then R³ represents Et or Me;
otherwise R³ represents Et, Me or Br.

19. The method of claim 16 wherein R¹ represents phenyl optionally substituted with one or two substituents independently selected from Me, OMe, CF₃, F and NMe₂, or phenyl substituted with either a) one Cl substituent at the 4-position, or b) two Cl substituents at the 3- and 4-positions.

20. The method of claim 16 wherein R² represents CF₃, or C₁₋₄alkyl.

21. The method of claim 16 or a pharmaceutically acceptable salt thereof wherein R³ represents Et.

22. The method of claim 16 wherein said compound is Formula (IC) or a pharmaceutically acceptable salt thereof: 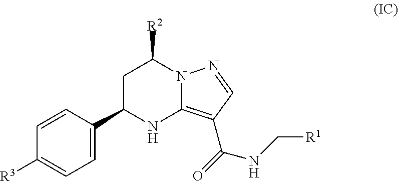
wherein
R¹ represents a group selected from:
(i) phenyl optionally substituted with one or two substituents independently selected from Me, OMe, CF₃, F, Cl and NMe₂;
(ii) furanyl, thiophenyl, pyrrolyl, pyridyl, cyclohexyl or naphthyl, each of which is optionally substituted with one or two substituents independently selected from Me, OMe, CF₃, F, Cl and NMe₂;
(iii) benzo[1,3]dioxo5-yl: 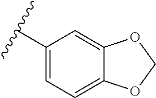 [SMILES: *c1ccc2c(c1)OCO2]
and
(iv) 2,3-dihydrobenzo[1,4]dioxin-6-yl: 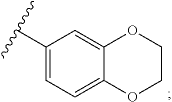 [SMILES: *c1ccc2c(c1)OCCO2]
R² represents CF₃, C₁₋₄alkyl, or CHF₂; and
R³ represents Et, Me, Br or OMe.

23. The method as claimed in claim 16, wherein the mammal is a human.

24. A method of treatment of tuberculosis in mammals, which method comprises the administration to a mammal in need of such treatment of an effective amount of a pharmaceutical composition comprising a compound, or a pharmaceutically acceptable salt thereof according to claim 16 and one or more pharmaceutically acceptable carriers, excipients or diluents.

25. The method of claim 24 wherein said mammal is a human.

26. A method of treatment of tuberculosis in mammals, which method comprises the administration to a mammal in need of such treatment of an effective amount of a combination comprising a compound of claim 16 or pharmaceutically acceptable salt thereof, together with one or more additional therapeutic agents.

27. The method as claimed in claim 26, wherein the one or more additional therapeutic agent is an anti-tuberculosis agent.

28. The method of claim 26 wherein said mammal is a human.

29. A compound of Formula (IB) or a pharmaceutically acceptable salt thereof: 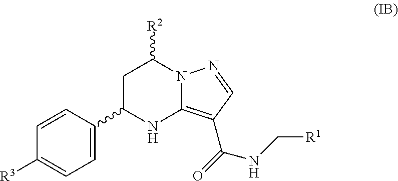
wherein:
R¹ represents a group selected from:
i) phenyl optionally substituted with one or two substituents independently selected from Me, CF₃, F and NMe₂ or phenyl substituted with either a) one Cl substituent at the 4-position, or b) two Cl substituents at the 3- and 4-positions;
ii) furanyl, pyrrolyl, pyridyl or naphthyl, each of which is optionally substituted with one or two substituents independently selected from Me, OMe, CF₃, F, Cl and NMe₂; or cyclohexyl or thiophenyl, each of which is substituted with one or two substituents independently selected from Me, OMe, CF₃, F, Cl and NMe₂;
iii) 2,3-dihydrobenzo[1,4]dioxin-6-yl: 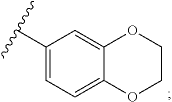 [SMILES: *c1ccc2c(c1)OCCO2]
R² represents CF₃, C₁₋₄alkyl, or CHF₂;
with the proviso that when R¹ represents optionally substituted furanyl, pyrrolyl, pyridyl or naphthyl, or substituted thiophenyl, or when R² represents CHF₂, then R³ represents Et; and
when R¹ represents substituted cyclohexyl, then R³ represents Et or Me;
otherwise R³ represents Et, Me or Br.

30. The compound of claim 29 or a pharmaceutically acceptable salt thereof wherein R¹ represents phenyl optionally substituted with one or two substituents independently selected from Me, OMe, CF₃, F and NMe₂, or phenyl substituted with either a) one Cl substituent at the 4-position, or b) two Cl substituents at the 3- and 4-positions.

31. The compound of claim 29 wherein R² represents CF₃, or C₁₋₄alkyl.

32. The compound of claim 29 or a pharmaceutically acceptable salt thereof wherein R³ represents Et.

33. The compound of claim 29 having Formula (IC) or a pharmaceutically acceptable salt thereof: 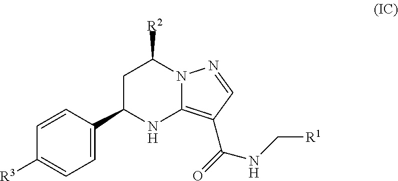
wherein R¹ represents a group selected from:
i) phenyl optionally substituted with one or two substituents independently selected from Me, OMe, CF₃, F, Cl and NMe₂:
ii) furanyl, thiophenyl, pyrrolyl, pyridyl, cyclohexyl or naphthyl, each of which is optionally substituted with one or two substituents independently selected from Me, OMe, CF₃, F, Cl and NMe₂;
iii) benzo[1,3]dioxo5-yl: 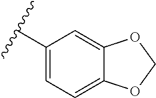 [SMILES: *c1ccc2c(c1)OCO2]
and
iv) 2,3-dihydrobenzo[1,4]dioxin-6-yl: 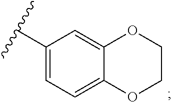 [SMILES: *c1ccc2c(c1)OCCO2]
R² represents CF₃, C₁₋₄alkyl, or CHF₂; and
R³ represents Et, Me, Br or OMe.

34. A pharmaceutical composition comprising a compound, or a pharmaceutically acceptable salt thereof according to claim 29 and one or more pharmaceutically acceptable carriers, excipients or diluents.

35. A combination comprising a compound of claim 29 or pharmaceutically acceptable salt thereof, together with one or more additional therapeutic agents.

36. The combination as claimed in claim 35, wherein the one or more additional therapeutic agents is an anti-tuberculosis agent.
